(19) United States
(12) Patent Application Publication
(10) Pub. No.: US 2026/0365602 A1
(43) Pub. Date: Nov. 19, 2026

(54) PROCESSES FOR PRODUCING RADICALLY-FUNCTIONALIZED PIBSA PRODUCT DERVIATIVES AND COMPOSITIONS COMPRISING SAME

(71) Applicant: The Lubrizol Corporation, Wickliffe, OH (US)

(72) Inventors: Nicolas Proust, Painesville, OH (US); Patrick Newby, Mentor, OH (US); Allison M. Williamson, Chardon, OH (US); Parmar Dixit, Long Eaton (GB); John R. Johnson, Bainbridge Township, OH (US); Binbin Guo, Sagamore Hills, OH (US); Joshua Armbrecht, Avon, OH (US); Paul R. Stevenson, Belper (GB)

(73) Assignee: The Lubrizol Corporation, Wickliffe, OH (US)

(21) Appl. No.: 19/099,980

(22) PCT Filed: Aug. 4, 2023
(86) PCT No.: PCT/US2023/029451

§ 371 Date: Jan. 30, 2025

Related U.S. Application Data
(60) Provisional application No. 63/395,361, filed on Aug. 5, 2022.

(51) Int. Cl. C07C 29/09; C10M 129/72

(52) U.S. Cl. CPC C07C 29/09; C10M 129/72; C10M 2207/282

(57) ABSTRACT

A lubricant composition including: (a) an oil of lubricating viscosity; and (b) a succinimide, succinimide, or succinate ester obtained by reacting: (i) an amine or an alcohol; with (ii) a reaction product obtained by a process comprising: (A) reacting polyisobutylene with a radical initiator and an ethylenically unsaturated acylating agent at a reaction temperature of from 150 °C to 225 °C to produce the reaction product; (B) wherein the polyisobutylene is brought to the reaction temperature prior to addition of the radical initiator; and (C) wherein the ethylenically unsaturated acylating agent is added at any time prior to or during addition of the radical initiator. Also, processes for producing reaction products which include such succinimides, succinimides, or succinate esters; methods of using such reaction products in lubricant compositions, uses of such lubricant compositions, and methods of using such lubricant compositions.

The disclosed technology relates to processes for producing reaction products of polyisobutylene with an initiator and acylating agent, derivatives of the acylated polyisobutylene comprised in the such reaction products, and the use of these derivatives in lubricant compositions.

Acylated polyisobutylenes, such as polyisobutylene succinic anhydrides/acids (“PIBSA”), are used as chemical intermediates in producing a vast number of chemical compositions. As used herein, the terms “polyisobutylene succinic anhydride”, “polyisobutylene succinic acid”, “polyisobutylene succinic anhydride/acid”, and/or “PIBSA” mean any molecule (or composition comprising such molecules) which includes a polyisobutylene portion bonded (directly or indirectly) to a succinic anhydride/acid unit. Known methods for producing PIBSA may require hazardous chemicals, high temperatures, long reaction times, and/or high energy demands, and may result in by-products and/or impurities (resulting in poor color and/or dark specks/resins, which may require filtration of the product) which reduce the possible markets for the products and/or require undesirable reaction equipment maintenance.

For example, polyisobutylene which contains high levels of tri- and tetra-substituted olefin end groups may be reacted with maleic anhydride in the presence of chlorine to form a desired PIBSA product. The drawbacks of this process include the need to use chlorine as a promoter, which often leads to some incorporation of chlorine into the final PIBSA product, and the formation of insoluble colored impurities in the product. The formation of these insoluble impurities impacts the manufacturing throughput as these solids can agglomerate within the reaction tanks, which results in increased maintenance down time in order to clean reactor tanks to remove these insoluble solids.

As another example, polyisobutylene that contains high levels of terminal vinylidene olefin end groups may be reacted with maleic anhydride in the presence of heat to form a desired PIBSA product. In a commercial manufacturing facility, this transformation has several drawbacks, including high reaction temperatures which are required to form the desired product, long reaction times, and the potential to promote decomposition of the acylating agent prior to the desired reaction taking place. One undesired side effect of these processing conditions is that the products can become visibly colored (typically orange/light brown).

One alternative to these illustrative approaches includes reacting high vinylidene polyisobutylene with maleic anhydride at lower reaction temperatures in the presence of a radical initiator. U.S. Pat. No. 5,112,507 teaches that high vinylidene polyisobutylene reacted with maleic anhydride in the presence of a radical initiator and reaction solvent forms a PIBSA product that is different in structure both from the PIBSA formed by the thermal process and PIBSA prepared by the chlorination process. Under certain reaction conditions, highly reactive polyisobutylene and maleic anhydride form an alternating copolymer where a monomeric unit is represented by the following general formula:

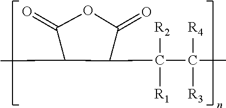

The degree of polymerization is represented by n, and indicates the number of repeat units present in the copolymer. Reaction temperatures were influenced by solvent selection, as most reactions were run at the reflux temperature for the solvent indicated. Reactions were run between 40 °C and 140 °C.

U.S. Pat. Nos. 5,565,528 and 5,616,668 teach that the desired reaction products can be formed in the absence of solvent at slightly higher reaction temperatures than those taught in U.S. Pat. No. 5,112,507. However, long reaction times can promote color formation in the product, and reduces manufacturing throughput on the commercial production scale.

The disclosed technology, therefore, in certain embodiments, provides a process for producing PIBSA which provides improvements in at least one of the above-mentioned shortcomings of known processes, and may provide more desirable PIBSA reaction products, perhaps resulting in higher quality reaction products and/or reduced reaction equipment maintenance. For example, the subject matter described herein may allow production of PIBSA product at significantly reduced time cycles and/or may minimize the undesired maleic anhydride decomposition that can lead to the formation of colored impurities.

Further, the disclosed technology provides processes which utilize the improved PIBSA reaction products to produce derivatives thereof, uses of such derivatives in lubricating compositions, methods of using such derivatives in lubricating compositions, lubricating compositions comprising the derivatives, and methods of lubricating which utilize the lubricating compositions.

The subject matter disclosed herein provides a lubricant composition comprising: (a) an oil of lubricating viscosity; and (b) succinimide, succinamide, or succinate ester obtained by reacting: (i) an amine or an alcohol; with (ii) a radically-functionalized PIBSA product obtained by a process comprising: (A) reacting polyisobutylene with a radical initiator and an ethylenically unsaturated acylating agent at a reaction temperature of from 150 °C to 225 °C to produce the reaction product; (B) wherein the polyisobutylene is brought to the reaction temperature prior to addition of the radical initiator; and (C) wherein the ethylenically unsaturated acylating agent is added at any time prior to or during addition of the radical initiator.

The subject matter disclosed herein also provide a process for producing a reaction product, the process comprising: reacting a radically-functionalized PIBSA product with an amine or an alcohol to form the reaction product; wherein the reaction product comprises at least one of a succinimide, a succinamide, or a succinate ester.

The following embodiments of the present subject matter are contemplated:
1. A lubricant composition comprising: (a) an oil of lubricating viscosity; and (b) a succinimide, succinamide, or succinate ester obtained by reacting: (i) an amine or an alcohol; with (ii) a radically-functionalized PIBSA product obtained by a process comprising: (A) reacting polyisobutylene with a radical initiator and an ethylenically unsaturated acylating agent at a reaction temperature of from 150 °C to 225 °C to produce the reaction product; (B) wherein the polyisobutylene is brought to the reaction temperature prior to addition of the radical initiator; and (C) wherein the ethylenically unsaturated acylating agent is added at any time prior to or during addition of the radical initiator.
2. The lubricant composition of embodiment 1, wherein the amine is a polyamine.
3. The lubricant composition of embodiment 2, wherein the polyamine contains at least one >N—H group per molecule.
4. The lubricant composition of either embodiment 2 or embodiment 3, wherein the polyamine is an aliphatic polyamine.
5. The lubricant composition of embodiment 4, wherein the aliphatic polyamine is at least one of an ethylenepolyamine, a propylenepolyamine, or a butylenepolyamine.
6. The lubricant composition of embodiment 5, wherein the ethylenepolyamine comprises at least one of ethylenediamine, diethylenetriamine, triethylenetetramine, tetraethylenepentamine, pentaethylenehexamine, or polyamine still bottoms.
7. The lubricant composition of any one of embodiments 1 to 6, wherein the alcohol is a polyhydric aliphatic alcohol.
8. The lubricant composition of embodiment 7, wherein the polyhydric aliphatic alcohol is chosen from glycerol, pentaerythritol, or sorbitol.
9. The lubricant composition of any one of embodiments 1 to 8, wherein the succinate ester obtained is further reacted with an amine.
10. The lubricant composition of embodiment 9, wherein the amine is an aliphatic amine.
11. The lubricant composition of embodiment 10, wherein the aliphatic amine is an aliphatic polyamine.
12. The lubricant composition of embodiment 11, wherein the aliphatic polyamine is at least one of an ethylenepolyamine, a propylenepolyamine, or a butylenepolyamine.
13. The lubricant composition of embodiment 12, wherein the ethylenepolyamine comprises at least one of ethylenediamine, diethylenetriamine, triethylenetetramine, tetraethylenepentamine, pentaethylenehexamine, or polyamine still bottoms.
14. The lubricant composition of any one of embodiments 1 to 13, wherein the succinimide, succinamide, or succinate ester is post-treated with a post-treating agent to produce a post-treated succinimide, a post-treated succinimide, or a post-treated succinate ester.
15. The lubricant composition of embodiment 14, wherein the post-treating agent comprises at least one of urea, thiourea, carbon disulfide, aldehydes, ketones, carboxylic acids, hydrocarbon-substituted succinic anhydrides, nitriles, epoxides, boron compounds, phosphorus compounds, 2,5-dimercaptothiadiazole, or an aromatic diacid having acid groups in 1,3 or 1,4 positions on a benzene ring.
16. The lubricant composition of embodiment 15, wherein the phosphorus compound is a phosphorus acids or anhydrides.
17. The lubricant composition of either embodiment 15 or embodiment 16, wherein the aromatic diacid having acid groups in 1,3 or 1,4 positions on a benzene ring is terephthalic acid.
18. The lubricant composition of any one of embodiments 14 to 17, wherein the post-treated succinimide or post-treated succinamide is a borated succinimide or a borated succinamide.
19. The lubricant composition of any one of embodiments 1 to 18, further comprising at least one additional additive comprising at least one of detergents, dispersants, friction modifiers, antiwear agents, extreme pressure agents, corrosion inhibitors, viscosity modifiers, antifoam agents, demulsifiers, or seal swell agents.
20. A method of lubricating a mechanical device comprising supplying the lubricant composition of any one of embodiments 1 to 19 to the mechanical device.
21. The method of embodiment 20, wherein the mechanical device is the crankcase of an internal combustion engine, a gear, a gearbox, an axle gear, or a transmission.
22. The method of embodiment 21, wherein the internal combustion engine is a spark ignited engine, or a compression ignited engine.
23. The method of embodiment 21, wherein the gear, the gearbox, or the axle gear comprises a differential, or a limited slip differential.
24. The method of embodiment 21, wherein the transmission is an automatic transmission.
25. The method of embodiment 24, wherein the automatic transmission comprises a continuously variable transmission, an infinitely variable transmission, a toroidal transmission, a continuously slipping torque converted clutch, a stepped automatic transmission, or a dual clutch transmission.
26. The method of embodiment 21, wherein the transmission is a manual transmission.
27. The method of embodiment 26, wherein the manual transmission is a farm tractor transmission.
28. A process for producing a reaction product, the process comprising: reacting a radically-functionalized PIBSA product with an amine or an alcohol to form the reaction product; wherein the reaction product comprises at least one of a succinimide, a succinamide, or a succinate ester.
29. The process of embodiment 28, wherein the amine is a polyamine.
30. The process of embodiment 29, wherein the polyamine contains at least one >N—H group per molecule.
31. The process of either embodiment 28 or embodiment 29, wherein the polyamine is an aliphatic polyamine.
32. The process of embodiment 31, wherein the aliphatic polyamine is at least one of an ethylenepolyamine, a propylenepolyamine, or a butylenepolyamine.
33. The process of embodiment 32, wherein the ethylenepolyamine comprises at least one of ethylenediamine, diethylenetriamine, triethylenetetramine, tetraethylenepentamine, pentaethylenehexamine, or polyamine still bottoms.
34. The process of any one of embodiments 28 to 33, wherein the alcohol is a polyhydric aliphatic alcohol.
35. The process of embodiment 34, wherein the polyhydric aliphatic alcohol is chosen from glycerol, pentaerythritol, or sorbitol.
36. The process of any one of embodiments 28 to 35, wherein the succinate ester obtained is further reacted with an amine.
37. The process of embodiment 36, wherein the amine is an aliphatic amine.
38. The process of embodiment 37, wherein the aliphatic amine is an aliphatic polyamine.
39. The process of embodiment 38, wherein the aliphatic polyamine is at least one of an ethylenepolyamine, a propylenepolyamine, or a butylenepolyamine.
40. The process of embodiment 39, wherein the ethylenepolyamine comprises at least one of ethylenediamine, diethylenetriamine, triethylenetetramine, tetraethylenepentamine, pentaethylenehexamine, or polyamine still bottoms.
41. The process of any one of embodiments 28 to 40, wherein the succinimide, succinamide or succinate ester is post-treated with a post-treating agent to produce a post-treated succinimide, a post-treated succinimide, or a post-treated succinate ester.
42. The process of embodiment 41, wherein the post-treating agent comprises at least one of urea, thiourea, carbon disulfide, aldehydes, ketones, carboxylic acids, hydrocarbon-substituted succinic anhydrides, nitriles, epoxides, boron compounds, phosphorus compounds, 2,5-dimercaptothiadiazole, or an aromatic diacid having acid groups in 1,3 or 1,4 positions on a benzene ring.
43. The process of embodiment 42, wherein the phosphorus compound is a phosphorus acids or anhydrides.
44. The process of either embodiment 42 or embodiment 43, wherein the aromatic diacid having acid groups in 1,3 or 1,4 positions on a benzene ring is terephthalic acid.
45. The process of any one of embodiments 41 to 44, wherein the post-treated succinimide or post-treated succinamide is a borated succinimide or a borated succinamide.
46. The process of any one of embodiments 28 to 45, wherein the radically-functionalized PIBSA product is a product of a process comprising: reacting polyisobutylene with a radical initiator and an ethylenically unsaturated acylating agent at a reaction temperature of from 150 °C to 225 °C to produce the product; wherein the polyisobutylene is brought to the reaction temperature prior to addition of the radical initiator; and wherein the ethylenically unsaturated acylating agent is added at any time prior to or during addition of the radical initiator.
47. The process of embodiment 46, wherein the ethylenically unsaturated acylating agent is combined with the polyisobutylene at the beginning of the process.
48. The process of embodiment 46, wherein the ethylenically unsaturated acylating agent is combined with the radical initiator for addition to the polyisobutylene after the polyisobutylene is brought to the reaction temperature.
49. The process of any one of embodiments 46 to 48, wherein the radically-functionalized PIBSA product comprises less than 15 weight percent of a material comprising two or more polyisobutylene units linked by one or more acylating agent units.
50. The process of any one of embodiments 46 to 49, wherein the polyisobutylene comprises an average of 3 to 56 isobutylene monomeric units per molecule.
51. The process of any one of embodiment 46 to 50, wherein the radical initiator comprises an organic peroxide initiator or a peracid initiator.
52. The process of embodiment 51, wherein the organic peroxide initiator comprises diglutaroyl, dilauroyl peroxide, benzoyl peroxide, dicumyl peroxide, di(tert butyl) peroxide, tertbutyl hydroperoxide, 3,4-dimethyl-3,4-diphenyl-hexane, tert-butylperoxy-2-ethylhexanoate, tert-butylperoxy-benzoate, tert-butylperoxy-pivalate, tert-butylperoxy-3,5,5-trimethyl-hexanoate, di(2,4-dichlorobenzoyl) peroxide, di(2-methylbenzoyl) peroxide, or di(4-tert-butylcyclohexyl) peroxydicarbonate.
53. The process of any one of embodiments 46 to 52, wherein the ethylenically unsaturated acylating agent comprises maleic acid, fumaric acid, itaconic acid, mesaconic acid, methylenemalonic acid, citraconic acid, maleic anhydride, itaconic anhydride, citraconic anhydride, or methylenemalonic anhydride.
54. The process of any one of embodiments 46 to 53, wherein the radically-functionalized PIBSA product comprises at least one compound of the following general formula I:

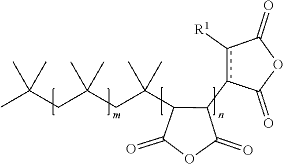

wherein: each R¹ independently represents: —(CH₂)—(C(CH₂))—(CH₃); H; or is not present; wherein when R¹ is not present, the dashed double bond represents a double bond, and when R¹ is present, the dashed double bond represents a single bond; each m independently is an integer from 1 to 52; and each n independently is 0, 1 or 2.
55. The process of embodiment 54, wherein the radically-functionalized PIBSA product comprises at least 30 mol % of the at least one compound of the general formula I.
56. The process of any one of embodiments 46 to 55, wherein the radically-functionalized PIBSA product comprises less than 40 weight percent unreacted polyisobutylene.
57. A composition comprising the reaction product of the process of any one of embodiments 28 to 57.
58. A lubricant composition comprising an oil of lubricating viscosity and the composition of embodiment 57.
59. The lubricant composition of embodiment 58, further comprising at least one additional additive comprising at least one of detergents, dispersants, friction modifiers, antiwear agents, extreme pressure agents, corrosion inhibitors, viscosity modifiers, antifoam agents, demulsifiers, or seal swell agents.
60. A method of lubricating a mechanical device comprising supplying the lubricant composition of either embodiment 58 or embodiment 59 to the mechanical device.
61. The method of embodiment 60, wherein the mechanical device is the crankcase of an internal combustion engine, a gear, a gearbox, an axle gear, or a transmission.
62. The method of embodiment 61, wherein the internal combustion engine is a spark ignited engine, or a compression ignited engine.
63. The method of embodiment 61, wherein the gear, the gearbox, or the axle gear comprises a differential, or a limited slip differential.
64. The method of embodiment 61, wherein the transmission is an automatic transmission.
65. The method of embodiment 64, wherein the automatic transmission comprises a continuously variable transmission, an infinitely variable transmission, a toroidal transmission, a continuously slipping torque converted clutch, a stepped automatic transmission, or a dual clutch transmission.
66. The method of embodiment 61, wherein the transmission is a manual transmission.
67. The method of embodiment 66, wherein the manual transmission is a farm tractor transmission.

Various features and embodiments of the present subject matter will be described below by way of non-limiting illustration.

As used herein, the term “condensation product” is intended to encompass esters, amides, imides and other such materials that may be prepared by a condensation reaction of an acid or a reactive equivalent of an acid (e.g., an acid halide, anhydride, or ester) with an alcohol or amine, irrespective of whether a condensation reaction is actually performed to lead directly to the product. Thus, for example, a particular ester may be prepared by a transesterification reaction rather than directly by a condensation reaction. The resulting product is still considered a condensation product.

The amount of each chemical component described herein is presented exclusive of any solvent or diluent oil, which may be customarily present in the commercial material, that is, on an active chemical basis, unless otherwise indicated. Unless otherwise indicated, each chemical or composition referred to herein should be interpreted as being a commercial grade material which may contain the isomers, by-products, derivatives, and other such materials which are normally understood to be present in the commercial grade.

As used herein, the term “hydrocarbyl” refers to a group having a carbon atom directly attached to the remainder of the molecule, where the group includes at least carbon and hydrogen atoms. If the hydrocarbyl group comprises more than one carbon atom, then those carbons need not necessarily be linked to each other. For example, at least two of the carbons may be linked via a suitable element or group. In various embodiments, the term “hydrocarbyl” refers to a group having a carbon atom directly attached to the remainder of the molecule, where the group consists of carbon, hydrogen, optionally one or more heteroatoms provided the heteroatoms do not alter the predominantly hydrocarbon nature of the substituent. The heteroatom may to link at least two of the carbons in the hydrocarbyl group, and optionally no more than two non-hydrocarbon substituents. Suitable heteroatoms will be apparent to those skilled in the art and include, for instance, sulphur, nitrogen, oxygen, phosphorus and silicon. Where the hydrocarbyl contains heteroatoms, optionally, no more than two heteroatoms will be present for every ten carbon atoms in the hydrocarbyl group. Suitable non-hydrocarbon substituents will also be apparent to those skilled in the art and include, for instance, halo, hydroxy, alkoxy, mercapto, alkylmercapto, nitro, nitroso, and sulphoxy.

Examples of hydrocarbyls within the context of the present technology therefore include: (i) hydrocarbon groups selected from aliphatic (e.g. alkyl or alkenyl), alicyclic (e.g. cycloalkyl, cycloalkenyl, cycloalkadienyl), and aromatic groups; (ii) substituted hydrocarbon groups, selected from hydrocarbon groups defined in (i) substituted with no more than two non-hydrocarbon substituents and/or one or more hydrocarbon substituents, the non-hydrocarbon substituents being selected from the group consisting of halo, hydroxy, alkoxy, mercapto, alkylmercapto, nitro, nitroso, and sulphoxy; and/or (iii) hetero-containing hydrocarbon groups, selected from hydrocarbon groups defined in (i) containing one or more heteroatom in the ring or chain, provided that the group has no more than two heteroatoms present for every ten carbon atoms in the group, the heteroatoms being selected from sulphur, nitrogen, oxygen, phosphorus and silicon. The hetero-containing hydrocarbon groups may be substituted with no more than two non-hydrocarbon substituents and/or one or more hydrocarbon substituents. In certain embodiments, the term “hydrocarbyl” refers to a group having a carbon atom directly attached to the remainder of the molecule, where the group consists of carbon and hydrogen atoms.

It is known that some of the materials described herein may interact in the final formulation, so that the components of the final formulation may be different from those that are initially added. For instance, metal ions (of, e.g., a detergent) may migrate to other acidic or anionic sites of other molecules. The products formed thereby, including the products formed upon employing the composition of the present subject matter in its intended use, may not be susceptible of easy description. Nevertheless, all such modifications and reaction products are included within the scope of the present subject matter; the present subject matter encompasses the composition prepared by admixing the components described herein.

As used herein, the indefinite article “a” !“an” is intended to mean one or more than one. As used herein, the phrase “at least one” means one or more than one of the following terms. Thus, “a”/“an” and “at least one” may be used interchangeably. For example “at least one of A, B or C” means that just one of A, B or C may be included, and any mixture of two or more of A, B and C may be included, in alternative embodiments.

As used herein, the term “substantially” means that a value of a given quantity is within ±10% of the stated value. In other embodiments, the value is within ±5% of the stated value. In other embodiments, the value is within ±2.5% of the stated value. In other embodiments, the value is within ±1% of the stated value.

As used herein, the term “substantially free of” means that a component does not include any intentional addition of the material which the component is “substantially free of”. For example, the component may include a material which the component is “substantially free of” at no more than impurity levels, which may be the result of incomplete chemical reactions and/or unintended/undesired (but perhaps unavoidable) reaction products.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

Number average molecular weights (Mn) described herein may be measured using gel permeation chromatography (GPC) using a Waters GPC 2000 equipped with a refractive index detector and Waters Empower™ data acquisition and analysis software. Samples were run against polystyrene calibration standards. The columns are polystyrene (PLgel, 5 micron, available from Agilent/Polymer Laboratories, Inc.). For the mobile phase, individual samples are dissolved in tetrahydrofuran and filtered with PTFE filters before they are injected into the GPC port. Waters GPC 2000 Operating Conditions: Injector, Column, and Pump/Solvent compartment temperatures, 40 °C; Autosampler Control: Run time, 40 minutes; Injection volume, 300 microliter; Pump, System pressure, −90 bars (maximum pressure limit, 270 bars; minimum pressure limit, 0 psi); Flow rate, 1.0 ml/minute; Differential Refractometer, Sensitivity, −16; Scale factor: 6.

Reaction Product Comprising a Succinimide, a Succinamide, and/or a Succinate Ester

Provided is a lubricant compositions comprising: (a) an oil of lubricating viscosity; and (b) a succinimide, succinimide, or succinate ester obtained by reacting: (i) an amine or an alcohol; with (ii) a radically-functionalized PIBSA product (as defined herein) obtained by a process comprising: (A) reacting polyisobutylene with a radical initiator and an ethylenically unsaturated acylating agent at a reaction temperature of from 150 °C to 225 °C to produce the reaction product; (B) wherein the polyisobutylene is brought to the reaction temperature prior to addition of the radical initiator; and (C) wherein the ethylenically unsaturated acylating agent is added at any time prior to or during addition of the radical initiator.

In certain embodiments, the amine is a polyamine. In certain embodiments, the polyamine contains at least one >N—H group per molecule. In certain embodiments, the polyamine is an aliphatic polyamine. In certain embodiments, the aliphatic polyamine is at least one of an ethylenepolyamine, a propylenepolyamine, or a butylenepolyamine. In certain embodiments, the ethylenepolyamine comprises at least one of ethylenediamine, diethylenetriamine, triethylenetetramine, tetraethylenepentamine, pentaethylenehexamine, or polyamine still bottoms.

In certain embodiments, the alcohol is a polyhydric aliphatic alcohol. In certain embodiments, the polyhydric aliphatic alcohol is chosen from glycerol, pentaerythritol, or sorbitol.

In certain embodiments, the succinate ester obtained is further reacted with an amine. In certain embodiments, the amine is an aliphatic amine. In certain embodiments, the aliphatic amine is an aliphatic polyamine. In certain embodiments, the aliphatic polyamine is at least one of an ethylenepolyamine, a propylenepolyamine, or a butylenepolyamine. In certain embodiments, the ethylenepolyamine comprises at least one of ethylenediamine, diethylenetriamine, triethylenetetramine, tetraethylenepentamine, pentaethylenehexamine, or polyamine still bottoms.

In certain embodiments, the succinimide, succinamide or succinate ester is post-treated with a post-treating agent to produce a post-treated succinimide, a post-treated succinimide, or a post-treated succinate ester. In certain embodiments, the post-treating agent comprises at least one of urea, thiourea, carbon disulfide, aldehydes, ketones, carboxylic acids, hydrocarbon-substituted succinic anhydrides, nitriles, epoxides, boron compounds, phosphorus compounds, 2,5-dimercaptothiadiazole, or an aromatic diacid having acid groups in 1,3 or 1,4 positions on a benzene ring. In certain embodiments, the phosphorus compound is a phosphorus acids or anhydrides. In certain embodiments, the aromatic diacid having acid groups in 1,3 or 1,4 positions on a benzene ring is terephthalic acid. In certain embodiments, the post-treated succinimide or post-treated succinamide is a borated succinimide or a borated succinamide.

Further provided are processes for producing a reaction product, the processes comprising: reacting a radically-functionalized PIBSA product with an amine or an alcohol to form the reaction product; wherein the reaction product comprises at least one of a succinimide, a succinamide, or a succinate ester. In certain embodiments, provided is a composition comprising a reaction product of such processes.

In certain embodiments, the amine is a polyamine. In certain embodiments, the polyamine contains at least one >N—H group per molecule. In certain embodiments, the polyamine is an aliphatic polyamine. In certain embodiments, the aliphatic polyamine is at least one of an ethylenepolyamine, a propylenepolyamine, or a butylenepolyamine. In certain embodiments, the ethylenepolyamine comprises at least one of ethylenediamine, diethylenetriamine, triethylenetetramine, tetraethylenepentamine, pentaethylenehexamine, or polyamine still bottoms.

In certain embodiments, the alcohol is a polyhydric aliphatic alcohol. In certain embodiments, the polyhydric aliphatic alcohol is chosen from glycerol, pentaerythritol, or sorbitol.

In certain embodiments, the succinate ester obtained is further reacted with an amine. In certain embodiments, the amine is an aliphatic amine. In certain embodiments, the aliphatic amine is an aliphatic polyamine. In certain embodiments, the aliphatic polyamine is at least one of an ethylenepolyamine, a propylenepolyamine, or a butylenepolyamine. In certain embodiments, the ethylenepolyamine comprises at least one of ethylenediamine, diethylenetriamine, triethylenetetramine, tetraethylenepentamine, pentaethylenehexamine, or polyamine still bottoms.

In certain embodiments, the succinimide, succinamide or succinate ester is post-treated with a post-treating agent to produce a post-treated succinimide, a post-treated succinimide, or a post-treated succinate ester. In certain embodiments, the post-treating agent comprises at least one of urea, thiourea, carbon disulfide, aldehydes, ketones, carboxylic acids, hydrocarbon-substituted succinic anhydrides, nitriles, epoxides, boron compounds, phosphorus compounds, 2,5-dimercaptothiadiazole, or an aromatic diacid having acid groups in 1,3 or 1,4 positions on a benzene ring. In certain embodiments, the phosphorus compound is a phosphorus acids or anhydrides. In certain embodiments, the aromatic diacid having acid groups in 1,3 or 1,4 positions on a benzene ring is terephthalic acid. In certain embodiments, the post-treated succinimide or post-treated succinamide is a borated succinimide or a borated succinamide.

### Radically-Functionalized PIBSA Product

In certain embodiments, the radically-functionalized polyisobutylene succinic anhydrides/acids (“PIBSA”) product is a product of a process comprising: reacting polyisobutylene with a radical initiator and an ethylenically unsaturated acylating agent at a reaction temperature of from 150 °C to 225 °C to produce the product; wherein the polyisobutylene is brought to the reaction temperature prior to addition of the radical initiator; and wherein the ethylenically unsaturated acylating agent is added at any time prior to or during addition of the radical initiator. As such, the term “radically-functionalized PIBSA product”, as used herein, is intended to refer to the product of this reaction, optionally including the embodiments described below. The radically-functionalized PIBSA product therefore may include other products of such reactions in addition to the intended and/or desired PIBSA product, as would be understood by a person of ordinary skill in the art.

In certain embodiments, the ethylenically unsaturated acylating agent may be combined with the polyisobutylene at the beginning of the process. In certain embodiments, the ethylenically unsaturated acylating agent may be combined with the radical initiator for addition to the polyisobutylene after the polyisobutylene is brought to the reaction temperature. In certain embodiments, the process for producing the radically-functionalized PIBSA product may comprise reacting polyisobutylene (such as high-vinylidene polyisobutylene) with a radical initiator and an ethylenically unsaturated acylating agent at a reaction temperature of from 150 °C to 225 °C to produce the radically-functionalized PIBSA product, wherein the polyisobutylene is brought to the reaction temperature prior to addition of the radical initiator and the ethylenically unsaturated acylating agent.

As used herein, the term “high-vinylidene polyisobutylene” means a compound including at least 55 (such as at 60, at least 65, at least 70, at least 75, or at least 80) weight percent alpha-methyl-vinylidene polyisobutylene. In certain embodiments, the high-vinylidene polyisobutylene may comprise from 55 to 80 (such as from 55 to 75, from 55 to 70, from 55 to 65, from 55 to 60, from 60 to 80, from 60 to 75, from 60 to 70, from 60 to 65, from 65 to 80, from 65 to 75, from 65 to 70, from 70 to 80, from 70 to 75, or from 75 to 80) weight percent alpha-methyl-vinylidene polyisobutylene. High vinylidene polyisobutylenes are well known, are known to be highly reactive relative to other polyisobutylenes, and are useful as chemical intermediates in numerous chemical synthesis processes.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a temperature of from 150 °C to 225 °C to produce the radically-functionalized PIBSA product.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a reaction temperature of from 150 °C to 225 °C to produce the radically-functionalized PIBSA product, wherein each of the two or more additions comprises at least 5 weight percent of the total amount of the radical initiator and/or at least 5 weight percent of the total amount of the ethylenically unsaturated acylating agent to be reacted with the polyisobutylene.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a reaction temperature of from 150 °C to 225 °C to produce the radically-functionalized PIBSA product, wherein each of the two or more additions are separated in time by at least twice the half-life of the radical initiator.

In certain embodiments, the process for producing the radically-functionalized PIBSA product may comprise: reacting polyisobutylene (such as high-vinylidene polyisobutylene) with a radical initiator and an ethylenically unsaturated acylating agent in two or more additions at a reaction temperature of from 150 °C to 225 °C to produce the radically-functionalized PIBSA product; wherein each of the two or more additions comprises at least 5 weight percent of the total amount of the radical initiator and/or at least 5 weight percent of the total amount of the ethylenically unsaturated acylating agent to be reacted with the polyisobutylene; and wherein each of the two or more additions are separated in time by at least twice the half-life of the radical initiator.

In certain embodiments, the process for producing the radically-functionalized PIBSA product may comprise: reacting polyisobutylene (such as high-vinylidene polyisobutylene) with a radical initiator and an ethylenically unsaturated acylating agent at a temperature of from 150 °C to 225 °C to produce the radically-functionalized PIBSA product, wherein the polyisobutylene is brought to the reaction temperature in a continuous reactor, prior to commencing continuous addition of the radical initiator to the polyisobutylene in the continuous reactor; and wherein the ethylenically unsaturated acylating agent is added to the continuous reactor: (i) as a mixture with the polyisobutylene; (ii) as a mixture with the radical initiator; and/or (iii) continuously and separately from either the polyisobutylene or the radical initiator.

As compared with known processes for producing PIBSA, the processes for producing the radically-functionalized PIBSA product described above generally provide lower color products (e.g., free of dark specks/resins without filtration), ability to access a degree of functionality greater than 2, decreased energy consumption, reduced equipment maintenance, and/or decreased reaction times, as will become apparent in the detailed description and examples below. These and/or other features of the radically-functionalized PIBSA product are believed to contribute to the increased benefits of the reaction product comprising quaternary ammonium salts described herein.

In certain embodiments, the reaction temperature utilized in the processes described herein to produce the radically-functionalized PIBSA product may be from 155 °C to 225 °C, from 160 °C to 225 °C, from 165 °C to 225 °C, from 166 °C to 225 °C, from 167 °C to 225 °C, from 168 °C to 225 °C, from 169 °C to 225 °C, from 170 °C to 225 °C, from 171 °C to 225 °C, from 172 °C to 225 °C, from 173 °C to 225 °C, from 174 °C to 225 °C, from 175 °C to 225 °C, from 150 °C to 220 °C, from 155 °C to 220 °C, from 160 °C to 220 °C, from 165 °C to 220 °C, from 166 °C to 220 °C, from 167 °C to 220 °C, from 168 °C to 220 °C, from 169 °C to 220 °C, from 170 °C to 220 °C, from 171 °C to 220 °C, from 172 °C to 220 °C, from 173 °C to 220 °C, from 174 °C to 220 °C, from 175 °C to 220 °C, from 150 °C to 215 °C, from 155 °C to 215 °C, from 160 °C to 215 °C, from 165 °C to 215 °C, from 166 °C to 215 °C, from 167 °C to 215 °C, from 168 °C to 215 °C, from 169 °C to 215 °C, from 170 °C to 215 °C, from 171 °C to 215 °C, from 172 °C to 215 °C, from 173 °C to 215 °C, from 174 °C to 215 °C, from 175 °C to 215 °C, from 150 °C to 210 °C, from 155 °C to 210 °C, from 160 °C to 210 °C, from 165 °C to 210 °C, from 166 °C to 210 °C, from 167 °C to 210 °C, from 168 °C to 210 °C, from 169 °C to 210 °C, from 170 °C to 210 °C, from 171 °C to 210 °C, from 172 °C to 210 °C, from 173 °C to 210 °C, from 174 °C to 210 °C, from 175 °C to 210 °C, from 150 °C to 205 °C, from 155 °C to 205 °C, from 160 °C to 205 °C, from 165 °C to 205 °C, from 166 °C to 205 °C, from 167 °C to 205 °C, from 168 °C to 205 °C, from 169 °C to 205 °C, from 170 °C to 205 °C, from 171 °C to 205 °C, from 172 °C to 205 °C, from 173 °C to 205 °C, from 174 °C to 205 °C, from 175 °C to 205 °C, from 150 °C to 200 °C, from 155 °C to 200 °C, from 160 °C to 200 °C, from 165 °C to 200 °C, from 166 °C to 200 °C, from 167 °C to 200 °C, from 168 °C to 200 °C, from 169 °C to 200 °C, from 170 °C to 200 °C, from 171 °C to 200 °C, from 172 °C to 200 °C, from 173 °C to 200 °C, from 174 °C to 200 °C, from 175 °C to 200 °C, from 150 °C to 195 °C, from 155 °C to 195 °C, from 160 °C to 195 °C, from 165 °C to 195 °C, from 166 °C to 195 °C, from 167 °C to 195 °C, from 168 °C to 195 °C, from 169 °C to 195 °C, from 170 °C to 195 °C, from 171 °C to 195 °C, from 172 °C to 195 °C, from 173 °C to 195 °C, from 174 °C to 195 °C, from 175 °C to 195 °C, from 150 °C to 190 °C, from 155 °C to 190 °C, from 160 °C to 190 °C, from 165 °C to 190 °C, from 166 °C to 190 °C, from 167 °C to 190 °C, from 168 °C to 190 °C, from 169 °C to 190 °C, from 170 °C to 190 °C, from 171 °C to 190 °C, from 172 °C to 190 °C, from 173 °C to 190 °C, from 174 °C to 190 °C, from 175 °C to 190 °C, from 150 °C to 185 °C, from 155 °C to 185 °C, from 160 °C to 185 °C, from 165 °C to 185 °C, from 166 °C to 185 °C, from 167 °C to 185 °C, from 168 °C to 185 °C, from 169 °C to 185 °C, from 170 °C to 185 °C, from 171 °C to 185 °C, from 172 °C to 185 °C, from 173 °C to 185 °C, from 174 °C to 185 °C, from 175 °C to 185 °C, from 150 °C to 180 °C, from 155 °C to 180 °C, from 160 °C to 180 °C, from 165 °C to 180 °C, from 166 °C to 180 °C, from 167 °C to 180 °C, from 168 °C to 180 °C, from 169 °C to 180 °C, from 170 °C to 180 °C, from 171 °C to 180 °C, from 172 °C to 180 °C, from 173 °C to 180 °C, from 174 °C to 180 °C, from 175 °C to 180 °C, from 150 °C to 175 °C, from 155 °C to 175 °C, from 160 °C to 175 °C, from 165 °C to 175 °C, from 166 °C to 175 °C, from 167 °C to 175 °C, from 168 °C to 175 °C, from 169 °C to 175 °C, from 170 °C to 175 °C, from 171 °C to 175 °C, from 172 °C to 175 °C, from 173 °C to 175 °C, from 174 °C to 175 °C, from 150 °C to 174 °C, from 155 °C to 174 °C, from 160 °C to 174 °C, from 165 °C to 174 °C, from 166 °C to 174 °C, from 167 °C to 174 °C, from 168 °C to 174 °C, from 169 °C to 174 °C, from 170 °C to 174 °C, from 171 °C to 174 °C, from 172 °C to 174 °C, from 173 °C to 174 °C, from 150 °C to 173 °C, from 155 °C to 173 °C, from 160 °C to 173 °C, from 165 °C to 173 °C, from 166 °C to 173 °C, from 167 °C to 173 °C, from 168 °C to 173 °C, from 169 °C to 173 °C, from 170 °C to 173 °C, from 171 °C to 173 °C, from 172 °C to 173 °C, from 150 °C to 172 °C, from 155 °C to 172 °C, from 160 °C to 172 °C, from 165 °C to 172 °C, from 166 °C to 172 °C, from 167 °C to 172 °C, from 168 °C to 172 °C, from 169 °C to 172 °C, from 170 °C to 172 °C, from 171 °C to 172 °C, from 150 °C to 171 °C, from 155 °C to 171 °C, from 160 °C to 171 °C, from 165 °C to 171 °C, from 166 °C to 171 °C, from 167 °C to 171 °C, from 168 °C to 171 °C, from 169 °C to 171 °C, from 170 °C to 171 °C, from 150 °C to 170 °C, from 155 °C to 170 °C, from 160 °C to 170 °C, from 165 °C to 170 °C, from 166 °C to 170 °C, from 167 °C to 170 °C, from 168 °C to 170 °C, or from 169 °C to 170 °C.

In certain embodiments, the total amount of radical initiator added during the processes to produce the radically-functionalized PIBSA product described herein may be from 0.01 to 1 (such as from 0.01 to 0.9, from 0.01 to 0.8, from 0.01 to 0.7, from 0.01 to 0.6, from 0.01 to 0.5, from 0.01 to 0.45, from 0.01 to 0.4, from 0.01 to 0.35, from 0.01 to 0.3, from 0.01 to 0.25, from 0.01 to 0.2, from 0.01 to 0.19, from 0.01 to 0.18, from 0.01 to 0.17, from 0.01 to 0.16, from 0.01 to 0.15, from 0.01 to 0.14, from 0.01 to 0.13, from 0.01 to 0.12, from 0.01 to 0.11, from 0.01 to 0.1, from 0.02 to 0.9, from 0.02 to 0.8, from 0.02 to 0.7, from 0.02 to 0.6, from 0.02 to 0.5, from 0.02 to 0.45, from 0.02 to 0.4, from 0.02 to 0.35, from 0.02 to 0.3, from 0.02 to 0.25, from 0.02 to 0.2, from 0.02 to 0.19, from 0.02 to 0.18, from 0.02 to 0.17, from 0.02 to 0.16, from 0.02 to 0.15, from 0.02 to 0.14, from 0.02 to 0.13, from 0.02 to 0.12, from 0.02 to 0.11, from 0.02 to 0.1, from 0.03 to 0.9, from 0.03 to 0.8, from 0.03 to 0.7, from 0.03 to 0.6, from 0.03 to 0.5, from 0.03 to 0.45, from 0.03 to 0.4, from 0.03 to 0.35, from 0.03 to 0.3, from 0.03 to 0.25, from 0.03 to 0.2, from 0.03 to 0.19, from 0.03 to 0.18, from 0.03 to 0.17, from 0.03 to 0.16, from 0.03 to 0.15, from 0.03 to 0.14, from 0.03 to 0.13, from 0.03 to 0.12, from 0.03 to 0.11, from 0.03 to 0.1, from 0.04 to 0.9, from 0.04 to 0.8, from 0.04 to 0.7, from 0.04 to 0.6, from 0.04 to 0.5, from 0.04 to 0.45, from 0.04 to 0.4, from 0.04 to 0.35, from 0.04 to 0.3, from 0.04 to 0.25, from 0.04 to 0.2, from 0.04 to 0.19, from 0.04 to 0.18, from 0.04 to 0.17, from 0.04 to 0.16, from 0.04 to 0.15, from 0.04 to 0.14, from 0.04 to 0.13, from 0.04 to 0.12, from 0.04 to 0.11, from 0.04 to 0.1, from 0.05 to 0.9, from 0.05 to 0.8, from 0.05 to 0.7, from 0.05 to 0.6, from 0.05 to 0.5, from 0.05 to 0.45, from 0.05 to 0.4, from 0.05 to 0.35, from 0.05 to 0.3, from 0.05 to 0.25, from 0.05 to 0.2, from 0.05 to 0.19, from 0.05 to 0.18, from 0.05 to 0.17, from 0.05 to 0.16, from 0.05 to 0.15, from 0.05 to 0.14, from 0.05 to 0.13, from 0.05 to 0.12, from 0.05 to 0.11, from 0.05 to 0.1, from 0.06 to 0.9, from 0.06 to 0.8, from 0.06 to 0.7, from 0.06 to 0.6, from 0.06 to 0.5, from 0.06 to 0.45, from 0.06 to 0.4, from 0.06 to 0.35, from 0.06 to 0.3, from 0.06 to 0.25, from 0.06 to 0.2, from 0.06 to 0.19, from 0.06 to 0.18, from 0.06 to 0.17, from 0.06 to 0.16, from 0.06 to 0.15, from 0.06 to 0.14, from 0.06 to 0.13, from 0.06 to 0.12, from 0.06 to 0.11, from 0.06 to 0.1, from 0.07 to 0.9, from 0.07 to 0.8, from 0.07 to 0.7, from 0.07 to 0.6, from 0.07 to 0.5, from 0.07 to 0.45, from 0.07 to 0.4, from 0.07 to 0.35, from 0.07 to 0.3, from 0.07 to 0.25, from 0.07 to 0.2, from 0.07 to 0.19, from 0.07 to 0.18, from 0.07 to 0.17, from 0.07 to 0.16, from 0.07 to 0.15, from 0.07 to 0.14, from 0.07 to 0.13, from 0.07 to 0.12, from 0.07 to 0.11, from 0.07 to 0.1, from 0.08 to 0.9, from 0.08 to 0.8, from 0.08 to 0.7, from 0.08 to 0.6, from 0.08 to 0.5, from 0.08 to 0.45, from 0.08 to 0.4, from 0.08 to 0.35, from 0.08 to 0.3, from 0.08 to 0.25, from 0.08 to 0.2, from 0.08 to 0.19, from 0.08 to 0.18, from 0.08 to 0.17, from 0.08 to 0.16, from 0.08 to 0.15, from 0.08 to 0.14, from 0.08 to 0.13, from 0.08 to 0.12, from 0.08 to 0.11, from 0.08 to 0.1, from 0.09 to 0.9, from 0.09 to 0.8, from 0.09 to 0.7, from 0.09 to 0.6, from 0.09 to 0.5, from 0.09 to 0.45, from 0.09 to 0.4, from 0.09 to 0.35, from 0.09 to 0.3, from 0.09 to 0.25, from 0.09 to 0.2, from 0.09 to 0.19, from 0.09 to 0.18, from 0.09 to 0.17, from 0.09 to 0.16, from 0.09 to 0.15, from 0.09 to 0.14, from 0.09 to 0.13, from 0.09 to 0.12, from 0.09 to 0.11, from 0.09 to 0.1, from 0.1 to 0.9, from 0.1 to 0.8, from 0.1 to 0.7, from 0.1 to 0.6, from 0.1 to 0.5, from 0.1 to 0.45, from 0.1 to 0.4, from 0.1 to 0.35, from 0.1 to 0.3, from 0.1 to 0.25, from 0.1 to 0.2, from 0.1 to 0.19, from 0.1 to 0.18, from 0.1 to 0.17, from 0.1 to 0.16, from 0.1 to 0.15, from 0.1 to 0.14, from 0.1 to 0.13, from 0.1 to 0.12, or from 0.1 to 0.11) equivalents, based on the amount of polyisobutylene being reacted.

In certain embodiments, the total amount of ethylenically unsaturated acylating agent added during the processes to produce the radically-functionalized PIBSA product described herein may be from 0.5 to 3 (such as from 0.5 to 2.9, from 0.5 to 2.8, from 0.5 to 2.7, from 0.5 to 2.6, from 0.5 to 2.5, from 0.5 to 2.4, from 0.5 to 2.3, from 0.5 to 2.4, from 0.5 to 2.3, from 0.5 to 2.2, from 0.5 to 2.1, from 0.5 to 2, from 0.5 to 1.9, from 0.5 to 1.8, from 0.5 to 1.7, from 0.5 to 1.6, from 0.5 to 1.5, from 0.5 to 1.4, from 0.5 to 1.3, from 0.5 to 1.2, from 0.5 to 1.1, from 0.5 to 1, from 0.6 to 3, from 0.6 to 2.9, from 0.6 to 2.8, from 0.6 to 2.7, from 0.6 to 2.6, from 0.6 to 2.5, from 0.6 to 2.4, from 0.6 to 2.3, from 0.6 to 2.4, from 0.6 to 2.3, from 0.6 to 2.2, from 0.6 to 2.1, from 0.6 to 2, from 0.6 to 1.9, from 0.6 to 1.8, from 0.6 to 1.7, from 0.6 to 1.6, from 0.6 to 1.5, from 0.6 to 1.4, from 0.6 to 1.3, from 0.6 to 1.2, from 0.6 to 1.1, from 0.6 to 1, from 0.7 to 3, from 0.7 to 2.9, from 0.7 to 2.8, from 0.7 to 2.7, from 0.7 to 2.6, from 0.7 to 2.5, from 0.7 to 2.4, from 0.7 to 2.3, from 0.7 to 2.4, from 0.7 to 2.3, from 0.7 to 2.2, from 0.7 to 2.1, from 0.7 to 2, from 0.7 to 1.9, from 0.7 to 1.8, from 0.7 to 1.7, from 0.7 to 1.6, from 0.7 to 1.5, from 0.7 to 1.4, from 0.7 to 1.3, from 0.7 to 1.2, from 0.7 to 1.1, from 0.7 to 1, from 0.8 to 3, from 0.8 to 2.9, from 0.8 to 2.8, from 0.8 to 2.7, from 0.8 to 2.6, from 0.8 to 2.5, from 0.8 to 2.4, from 0.8 to 2.3, from 0.8 to 2.4, from 0.8 to 2.3, from 0.8 to 2.2, from 0.8 to 2.1, from 0.8 to 2, from 0.8 to 1.9, from 0.8 to 1.8, from 0.8 to 1.7, from 0.8 to 1.6, from 0.8 to 1.5, from 0.8 to 1.4, from 0.8 to 1.3, from 0.8 to 1.2, from 0.8 to 1.1, from 0.8 to 1, from 0.9 to 3, from 0.9 to 2.9, from 0.9 to 2.8, from 0.9 to 2.7, from 0.9 to 2.6, from 0.9 to 2.5, from 0.9 to 2.4, from 0.9 to 2.3, from 0.9 to 2.2, from 0.9 to 2.1, from 0.9 to 2, from 0.9 to 1.9, from 0.9 to 1.8, from 0.9 to 1.7, from 0.9 to 1.6, from 0.9 to 1.5, from 0.9 to 1.4, from 0.9 to 1.3, from 0.9 to 1.2, from 0.9 to 1.1, from 0.9 to 1, from 1 to 3, from 1 to 2.9, from 1 to 2.8, from 1 to 2.7, from 1 to 2.6, from 1 to 2.5, from 1 to 2.4, from 1 to 2.3, from 1 to 2.2, from 1 to 2.1, from 1 to 2, from 1 to 1.9, from 1 to 1.8, from 1 to 1.7, from 1 to 1.6, from 1 to 1.5, from 1 to 1.4, from 1 to 1.3, from 1 to 1.2, from 1 to 1.1, from 1.1 to 3, from 1.1 to 2.9, from 1.1 to 2.8, from 1.1 to 2.7, from 1.1 to 2.6, from 1.1 to 2.5, from 1.1 to 2.4, from 1.1 to 2.3, from 1.1 to 2.2, from 1.1 to 2.1, from 1.1 to 2, from 1.1 to 1.9, from 1.1 to 1.8, from 1.1 to 1.7, from 1.1 to 1.6, from 1.1 to 1.5, from 1.1 to 1.4, from 1.1 to 1.3, from 1.1 to 1.2, from 1.2 to 3, from 1.2 to 2.9, from 1.2 to 2.8, from 1.2 to 2.7, from 1.2 to 2.6, from 1.2 to 2.5, from 1.2 to 2.4, from 1.2 to 2.3, from 1.2 to 2.2, from 1.2 to 2.1, from 1.2 to 2, from 1.2 to 1.9, from 1.2 to 1.8, from 1.2 to 1.7, from 1.2 to 1.6, from 1.2 to 1.5, from 1.2 to 1.4, from 1.2 to 1.3, from 1.3 to 3, from 1.3 to 2.9, from 1.3 to 2.8, from 1.3 to 2.7, from 1.3 to 2.6, from 1.3 to 2.5, from 1.3 to 2.4, from 1.3 to 2.3, from 1.3 to 2.2, from 1.3 to 2.1, from 1.3 to 2, from 1.3 to 1.9, from 1.3 to 1.8, from 1.3 to 1.7, from 1.3 to 1.6, from 1.3 to 1.5, from 1.3 to 1.4, from 1.4 to 3, from 1.4 to 2.9, from 1.4 to 2.8, from 1.4 to 2.7, from 1.4 to 2.6, from 1.4 to 2.5, from 1.4 to 2.4, from 1.4 to 2.3, from 1.4 to 2.2, from 1.4 to 2.1, from 1.4 to 2, from 1.4 to 1.9, from 1.4 to 1.8, from 1.4 to 1.7, from 1.4 to 1.6, from 1.4 to 1.5, from 1.5 to 3, from 1.5 to 2.9, from 1.5 to 2.8, from 1.5 to 2.7, from 1.5 to 2.6, from 1.5 to 2.5, from 1.5 to 2.4, from 1.5 to 2.3, from 1.5 to 2.2, from 1.5 to 2.1, from 1.5 to 2, from 1.5 to 1.9, from 1.5 to 1.8, from 1.5 to 1.7, from 1.5 to 1.6, from 1.6 to 3, from 1.6 to 2.9, from 1.6 to 2.8, from 1.6 to 2.7, from 1.6 to 2.6, from 1.6 to 2.5, from 1.6 to 2.4, from 1.6 to 2.3, from 1.6 to 2.2, from 1.6 to 2.1, from 1.6 to 2, from 1.6 to 1.9, from 1.6 to 1.8, from 1.6 to 1.7, from 1.7 to 3, from 1.7 to 2.9, from 1.7 to 2.8, from 1.7 to 2.7, from 1.7 to 2.6, from 1.7 to 2.5, from 1.7 to 2.4, from 1.7 to 2.3, from 1.7 to 2.2, from 1.7 to 2.1, from 1.7 to 2, from 1.7 to 1.9, from 1.7 to 1.8, from 1.8 to 3, from 1.8 to 2.9, from 1.8 to 2.8, from 1.8 to 2.7, from 1.8 to 2.6, from 1.8 to 2.5, from 1.8 to 2.4, from 1.8 to 2.3, from 1.8 to 2.2, from 1.8 to 2.1, from 1.8 to 2, from 1.8 to 1.9, from 1.9 to 3, from 1.9 to 2.9, from 1.9 to 2.8, from 1.9 to 2.7, from 1.9 to 2.6, from 1.9 to 2.5, from 1.9 to 2.4, from 1.9 to 2.3, from 1.9 to 2.2, from 1.9 to 2.1, from 1.9 to 2, from 2 to 3, from 2 to 2.9, from 2 to 2.8, from 2 to 2.7, from 2 to 2.6, from 2 to 2.5, from 2 to 2.4, from 2 to 2.3, from 2 to 2.2, from 2 to 2.1, from 2.1 to 3, from 2.1 to 2.9, from 2.1 to 2.8, from 2.1 to 2.7, from 2.1 to 2.6, from 2.1 to 2.5, from 2.1 to 2.4, from 2.1 to 2.3, from 2.1 to 2.2, from 2.2 to 3, from 2.2 to 2.9, from 2.2 to 2.8, from 2.2 to 2.7, from 2.2 to 2.6, from 2.2 to 2.5, from 2.2 to 2.4, from 2.2 to 2.3, from 2.3 to 3, from 2.3 to 2.9, from 2.3 to 2.8, from 2.3 to 2.7, from 2.3 to 2.6, from 2.3 to 2.5, from 2.3 to 2.4, from 2.4 to 3, from 2.4 to 2.9, from 2.4 to 2.8, from 2.4 to 2.7, from 2.4 to 2.6, from 2.4 to 2.5, from 2.5 to 3, from 2.5 to 2.9, from 2.5 to 2.8, from 2.5 to 2.7, or from 2.5 to 2.6) equivalents, based on the amount of polyisobutylene being reacted.

In certain embodiments, the radically-functionalized PIBSA product comprises less than 15 (such as less than 14, 13, 12, 11, 10, 9, 8, 7, 6, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) weight percent of a material comprising two or more polyisobutylene units linked by one or more acylating agent units. In certain embodiments, the radically-functionalized PIBSA product is substantially free of a material comprising two or more polyisobutylene units linked by one or more acylating agent units. In certain embodiments, the radically-functionalized PIBSA product comprises from 0 (such as greater than 0) to 15 (such as 14, 13, 12, 11, 10, 9, 8, 7, 6, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) weight percent of a material comprising two or more polyisobutylene units linked by one or more acylating agent units. In certain embodiments, the radically-functionalized PIBSA product comprises from 0 to 15 (such as from 0 to 14, from 0 to 13, from 0 to 12, from 0 to 11, from 0 to 10, from 0 to 9, from 0 to 8, from 0 to 7, from 0 to 6, from 0 to 5, from 0 to 4.5, from 0 to 4, from 0 to 3.5, from 0 to 3, from 0 to 2.5, from 0 to 2, from 0 to 1.9, from 0 to 1.8, from 0 to 1.7, from 0 to 1.6, from 0 to 1.5, from 0 to 1.4, from 0 to 1.3, from 0 to 1.2, from 0 to 1.1, from 0 to 1, from 0 to 0.9, from 0 to 0.8, from 0 to 0.7, from 0 to 0.6, from 0 to 0.5, from 0 to 0.4, from 0 to 0.3, from 0 to 0.2, from 0 to 0.1, from greater than 0 to 15, from greater than 0 to 14, from greater than 0 to 13, from greater than 0 to 12, from greater than 0 to 11, from greater than 0 to 10, from greater than 0 to 9, from greater than 0 to 8, from greater than 0 to 7, from greater than 0 to 6, from greater than 0 to 5, from greater than 0 to 4.5, from greater than 0 to 4, from greater than 0 to 3.5, from greater than 0 to 3, from greater than 0 to 2.5, from greater than 0 to 2, from greater than 0 to 1.9, from greater than 0 to 1.8, from greater than 0 to 1.7, from greater than 0 to 1.6, from greater than 0 to 1.5, from greater than 0 to 1.4, from greater than 0 to 1.3, from greater than 0 to 1.2, from greater than 0 to 1.1, from greater than 0 to 1, from greater than 0 to 0.9, from greater than 0 to 0.8, from greater than 0 to 0.7, from greater than 0 to 0.6, from greater than 0 to 0.5, from greater than 0 to 0.4, from greater than 0 to 0.3, from greater than 0 to 0.2, from greater than 0 to 0.1, from 0.1 to 15, from 0.1 to 14, from 0.1 to 13, from 0.1 to 12, from 0.1 to 11, from 0.1 to 10, from 0.1 to 9, from 0.1 to 8, from 0.1 to 7, from 0.1 to 6, from 0.1 to 5, from 0.1 to 4.5, from 0.1 to 4, from 0.1 to 3.5, from 0.1 to 3, from 0.1 to 2.5, from 0.1 to 2, from 0.1 to 1.9, from 0.1 to 1.8, from 0.1 to 1.7, from 0.1 to 1.6, from 0.1 to 1.5, from 0.1 to 1.4, from 0.1 to 1.3, from 0.1 to 1.2, from 0.1 to 1.1, from 0.1 to 1, from 0.1 to 0.9, from 0.1 to 0.8, from 0.1 to 0.7, from 0.1 to 0.6, from 0.1 to 0.5, from 0.1 to 0.4, from 0.1 to 0.3, from 0.1 to 0.2, from 0.2 to 15, from 0.2 to 14, from 0.2 to 13, from 0.2 to 12, from 0.2 to 11, from 0.2 to 10, from 0.2 to 9, from 0.2 to 8, from 0.2 to 7, from 0.2 to 6, from 0.2 to 5, from 0.2 to 4.5, from 0.2 to 4, from 0.2 to 3.5, from 0.2 to 3, from 0.2 to 2.5, from 0.2 to 2, from 0.2 to 1.9, from 0.2 to 1.8, from 0.2 to 1.7, from 0.2 to 1.6, from 0.2 to 1.5, from 0.2 to 1.4, from 0.2 to 1.3, from 0.2 to 1.2, from 0.2 to 1.1, from 0.2 to 1, from 0.2 to 0.9, from 0.2 to 0.8, from 0.2 to 0.7, from 0.2 to 0.6, from 0.2 to 0.5, from 0.2 to 0.4, from 0.2 to 0.3, from 0.3 to 15, from 0.3 to 14, from 0.3 to 13, from 0.3 to 12, from 0.3 to 11, from 0.3 to 10, from 0.3 to 9, from 0.3 to 8, from 0.3 to 7, from 0.3 to 6, from 0.3 to 5, from 0.3 to 4.5, from 0.3 to 4, from 0.3 to 3.5, from 0.3 to 3, from 0.3 to 2.5, from 0.3 to 2, from 0.3 to 1.9, from 0.3 to 1.8, from 0.3 to 1.7, from 0.3 to 1.6, from 0.3 to 1.5, from 0.3 to 1.4, from 0.3 to 1.3, from 0.3 to 1.2, from 0.3 to 1.1, from 0.3 to 1, from 0.3 to 0.9, from 0.3 to 0.8, from 0.3 to 0.7, from 0.3 to 0.6, from 0.3 to 0.5, from 0.3 to 0.4, from 0.4 to 15, from 0.4 to 14, from 0.4 to 13, from 0.4 to 12, from 0.4 to 11, from 0.4 to 10, from 0.4 to 9, from 0.4 to 8, from 0.4 to 7, from 0.4 to 6, from 0.4 to 5, from 0.4 to 4.5, from 0.4 to 4, from 0.4 to 3.5, from 0.4 to 3, from 0.4 to 2.5, from 0.4 to 2, from 0.4 to 1.9, from 0.4 to 1.8, from 0.4 to 1.7, from 0.4 to 1.6, from 0.4 to 1.5, from 0.4 to 1.4, from 0.4 to 1.3, from 0.4 to 1.2, from 0.4 to 1.1, from 0.4 to 1, from 0.4 to 0.9, from 0.4 to 0.8, from 0.4 to 0.7, from 0.4 to 0.6, from 0.4 to 0.5, from 0.5 to 15, from 0.5 to 14, from 0.5 to 13, from 0.5 to 12, from 0.5 to 11, from 0.5 to 10, from 0.5 to 9, from 0.5 to 8, from 0.5 to 7, from 0.5 to 6, from 0.5 to 5, from 0.5 to 4.5, from 0.5 to 4, from 0.5 to 3.5, from 0.5 to 3, from 0.5 to 2.5, from 0.5 to 2, from 0.5 to 1.9, from 0.5 to 1.8, from 0.5 to 1.7, from 0.5 to 1.6, from 0.5 to 1.5, from 0.5 to 1.4, from 0.5 to 1.3, from 0.5 to 1.2, from 0.5 to 1.1, from 0.5 to 1, from 0.5 to 0.9, from 0.5 to 0.8, from 0.5 to 0.7, from 0.5 to 0.6, from 0.6 to 15, from 0.6 to 14, from 0.6 to 13, from 0.6 to 12, from 0.6 to 11, from 0.6 to 10, from 0.6 to 9, from 0.6 to 8, from 0.6 to 7, from 0.6 to 6, from 0.6 to 5, from 0.6 to 4.5, from 0.6 to 4, from 0.6 to 3.5, from 0.6 to 3, from 0.6 to 2.5, from 0.6 to 2, from 0.6 to 1.9, from 0.6 to 1.8, from 0.6 to 1.7, from 0.6 to 1.6, from 0.6 to 1.5, from 0.6 to 1.4, from 0.6 to 1.3, from 0.6 to 1.2, from 0.6 to 1.1, from 0.6 to 1, from 0.6 to 0.9, from 0.6 to 0.8, from 0.6 to 0.7, from 0.7 to 15, from 0.7 to 14, from 0.7 to 13, from 0.7 to 12, from 0.7 to 11, from 0.7 to 10, from 0.7 to 9, from 0.7 to 8, from 0.7 to 7, from 0.7 to 6, from 0.7 to 5, from 0.7 to 4.5, from 0.7 to 4, from 0.7 to 3.5, from 0.7 to 3, from 0.7 to 2.5, from 0.7 to 2, from 0.7 to 1.9, from 0.7 to 1.8, from 0.7 to 1.7, from 0.7 to 1.6, from 0.7 to 1.5, from 0.7 to 1.4, from 0.7 to 1.3, from 0.7 to 1.2, from 0.7 to 1.1, from 0.7 to 1, from 0.7 to 0.9, from 0.7 to 0.8, from 0.8 to 15, from 0.8 to 14, from 0.8 to 13, from 0.8 to 12, from 0.8 to 11, from 0.8 to 10, from 0.8 to 9, from 0.8 to 8, from 0.8 to 7, from 0.8 to 6, from 0.8 to 5, from 0.8 to 4.5, from 0.8 to 4, from 0.8 to 3.5, from 0.8 to 3, from 0.8 to 2.5, from 0.8 to 2, from 0.8 to 1.9, from 0.8 to 1.8, from 0.8 to 1.7, from 0.8 to 1.6, from 0.8 to 1.5, from 0.8 to 1.4, from 0.8 to 1.3, from 0.8 to 1.2, from 0.8 to 1.1, from 0.8 to 1, from 0.8 to 0.9, from 0.9 to 15, from 0.9 to 14, from 0.9 to 13, from 0.9 to 12, from 0.9 to 11, from 0.9 to 10, from 0.9 to 9, from 0.9 to 8, from 0.9 to 7, from 0.9 to 6, from 0.9 to 5, from 0.9 to 4.5, from 0.9 to 4, from 0.9 to 3.5, from 0.9 to 3, from 0.9 to 2.5, from 0.9 to 2, from 0.9 to 1.9, from 0.9 to 1.8, from 0.9 to 1.7, from 0.9 to 1.6, from 0.9 to 1.5, from 0.9 to 1.4, from 0.9 to 1.3, from 0.9 to 1.2, from 0.9 to 1.1, from 0.9 to 1, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4.5, from 1 to 4, from 1 to 3.5, from 1 to 3, from 1 to 2.5, from 1 to 2, from 1 to 1.9, from 1 to 1.8, from 1 to 1.7, from 1 to 1.6, from 1 to 1.5, from 1 to 1.4, from 1 to 1.3, from 1 to 1.2, from 1 to 1.1, from 1.1 to 15, from 1.1 to 14, from 1.1 to 13, from 1.1 to 12, from 1.1 to 11, from 1.1 to 10, from 1.1 to 9, from 1.1 to 8, from 1.1 to 7, from 1.1 to 6, from 1.1 to 5, from 1.1 to 4.5, from 1.1 to 4, from 1.1 to 3.5, from 1.1 to 3, from 1.1 to 2.5, from 1.1 to 2, from 1.1 to 1.9, from 1.1 to 1.8, from 1.1 to 1.7, from 1.1 to 1.6, from 1.1 to 1.5, from 1.1 to 1.4, from 1.1 to 1.3, from 1.1 to 1.2, from 1.2 to 15, from 1.2 to 14, from 1.2 to 13, from 1.2 to 12, from 1.2 to 11, from 1.2 to 10, from 1.2 to 9, from 1.2 to 8, from 1.2 to 7, from 1.2 to 6, from 1.2 to 5, from 1.2 to 4.5, from 1.2 to 4, from 1.2 to 3.5, from 1.2 to 3, from 1.2 to 2.5, from 1.2 to 2, from 1.2 to 1.9, from 1.2 to 1.8, from 1.2 to 1.7, from 1.2 to 1.6, from 1.2 to 1.5, from 1.2 to 1.4, from 1.2 to 1.3, from 1.3 to 15, from 1.3 to 14, from 1.3 to 13, from 1.3 to 12, from 1.3 to 11, from 1.3 to 10, from 1.3 to 9, from 1.3 to 8, from 1.3 to 7, from 1.3 to 6, from 1.3 to 5, from 1.3 to 4.5, from 1.3 to 4, from 1.3 to 3.5, from 1.3 to 3, from 1.3 to 2.5, from 1.3 to 2, from 1.3 to 1.9, from 1.3 to 1.8, from 1.3 to 1.7, from 1.3 to 1.6, from 1.3 to 1.5, from 1.3 to 1.4, from 1.4 to 15, from 1.4 to 14, from 1.4 to 13, from 1.4 to 12, from 1.4 to 11, from 1.4 to 10, from 1.4 to 9, from 1.4 to 8, from 1.4 to 7, from 1.4 to 6, from 1.4 to 5, from 1.4 to 4.5, from 1.4 to 4, from 1.4 to 3.5, from 1.4 to 3, from 1.4 to 2.5, from 1.4 to 2, from 1.4 to 1.9, from 1.4 to 1.8, from 1.4 to 1.7, from 1.4 to 1.6, from 1.4 to 1.5, from 1.5 to 15, from 1.5 to 14, from 1.5 to 13, from 1.5 to 12, from 1.5 to 11, from 1.5 to 10, from 1.5 to 9, from 1.5 to 8, from 1.5 to 7, from 1.5 to 6, from 1.5 to 5, from 1.5 to 4.5, from 1.5 to 4, from 1.5 to 3.5, from 1.5 to 3, from 1.5 to 2.5, from 1.5 to 2, from 1.5 to 1.9, from 1.5 to 1.8, from 1.5 to 1.7, from 1.5 to 1.6, from 1.6 to 15, from 1.6 to 14, from 1.6 to 13, from 1.6 to 12, from 1.6 to 11, from 1.6 to 10, from 1.6 to 9, from 1.6 to 8, from 1.6 to 7, from 1.6 to 6, from 1.6 to 5, from 1.6 to 4.5, from 1.6 to 4, from 1.6 to 3.5, from 1.6 to 3, from 1.6 to 2.5, from 1.6 to 2, from 1.6 to 1.9, from 1.6 to 1.8, from 1.6 to 1.7, from 1.7 to 15, from 1.7 to 14, from 1.7 to 13, from 1.7 to 12, from 1.7 to 11, from 1.7 to 10, from 1.7 to 9, from 1.7 to 8, from 1.7 to 7, from 1.7 to 6, from 1.7 to 5, from 1.7 to 4.5, from 1.7 to 4, from 1.7 to 3.5, from 1.7 to 3, from 1.7 to 2.5, from 1.7 to 2, from 1.7 to 1.9, from 1.7 to 1.8, from 1.8 to 15, from 1.8 to 14, from 1.8 to 13, from 1.8 to 12, from 1.8 to 11, from 1.8 to 10, from 1.8 to 9, from 1.8 to 8, from 1.8 to 7, from 1.8 to 6, from 1.8 to 5, from 1.8 to 4.5, from 1.8 to 4, from 1.8 to 3.5, from 1.8 to 3, from 1.8 to 2.5, from 1.8 to 2, from 1.8 to 1.9, from 1.9 to 15, from 1.9 to 14, from 1.9 to 13, from 1.9 to 12, from 1.9 to 11, from 1.9 to 10, from 1.9 to 9, from 1.9 to 8, from 1.9 to 7, from 1.9 to 6, from 1.9 to 5, from 1.9 to 4.5, from 1.9 to 4, from 1.9 to 3.5, from 1.9 to 3, from 1.9 to 2.5, from 1.9 to 2, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4.5, from 2 to 4, from 2 to 3.5, from 2 to 3, from 2 to 2.5, from 2.5 to 15, from 2.5 to 14, from 2.5 to 13, from 2.5 to 12, from 2.5 to 11, from 2.5 to 10, from 2.5 to 9, from 2.5 to 8, from 2.5 to 7, from 2.5 to 6, from 2.5 to 5, from 2.5 to 4.5, from 2.5 to 4, from 2.5 to 3.5, from 2.5 to 3, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4.5, from 3 to 4, from 3 to 3.5, from 3.5 to 15, from 3.5 to 14, from 3.5 to 13, from 3.5 to 12, from 3.5 to 11, from 3.5 to 10, from 3.5 to 9, from 3.5 to 8, from 3.5 to 7, from 3.5 to 6, from 3.5 to 5, from 3.5 to 4.5, from 3.5 to 4, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 4 to 4.5, from 4.5 to 15, from 4.5 to 14, from 4.5 to 13, from 4.5 to 12, from 4.5 to 11, from 4.5 to 10, from 4.5 to 9, from 4.5 to 8, from 4.5 to 7, from 4.5 to 6, from 4.5 to 5, from 5 to 15, from 5 to 14, from 5 to 13, from 5 to 12, from 5 to 11, from 5 to 10, from 5 to 9, from 5 to 8, from 5 to 7, from 5 to 6, from 6 to 15, from 6 to 14, from 6 to 13, from 6 to 12, from 6 to 11, from 6 to 10, from 6 to 9, from 6 to 8, from 6 to 7, from 7 to 15, from 7 to 14, from 7 to 13, from 7 to 12, from 7 to 11, from 7 to 10, from 7 to 9, from 7 to 8, from 8 to 15, from 8 to 14, from 8 to 13, from 8 to 12, from 8 to 11, from 8 to 10, from 8 to 9, from 9 to 15, from 9 to 14, from 9 to 13, from 9 to 12, from 9 to 11, from 9 to 10, from 10 to 15, from 10 to 14, from 10 to 13, from 10 to 12, from 10 to 11, from 11 to 15, from 11 to 14, from 11 to 13, from 11 to 12, from 12 to 15, from 12 to 14, from 12 to 13, from 13 to 15, from 13 to 14, or from 14 to 15) weight percent of a material comprising two or more polyisobutylene units linked by one or more acylating agent units.

In certain embodiments, the polyisobutylene comprises an average of 3 to 56 isobutylene monomeric units per molecule. In certain embodiments, the polyisobutylene comprises an average of 3 to 55 isobutylene monomeric units per molecule. In certain embodiments, the polyisobutylene comprises an average of 3 to 54 isobutylene monomeric units per molecule. In certain embodiments, the polyisobutylene comprises an average of 3 to 53 isobutylene monomeric units per molecule. In certain embodiments, the polyisobutylene comprises an average of 3 to 52 isobutylene monomeric units per molecule. In certain embodiments, the polyisobutylene comprises an average of 3 to 51 isobutylene monomeric units per molecule. In certain embodiments, the polyisobutylene comprises an average of 3 to 50 isobutylene monomeric units per molecule. In certain embodiments, the polyisobutylene comprises an average of 3 to 48 isobutylene monomeric units per molecule. In certain embodiments, the polyisobutylene comprises an average of 3 to 46 isobutylene monomeric units per molecule. In certain embodiments, the polyisobutylene comprises an average of 3 to 44 isobutylene monomeric units per molecule. In certain embodiments, the polyisobutylene comprises an average of 3 to 42 isobutylene monomeric units per molecule. In certain embodiments, the polyisobutylene comprises an average of 3 to 40 isobutylene monomeric units per molecule. In certain embodiments, the polyisobutylene comprises an average of 3 to 38 isobutylene monomeric units per molecule. In certain embodiments, the polyisobutylene comprises an average of 3 to 36 isobutylene monomeric units per molecule. In certain embodiments, the polyisobutylene comprises an average of 3 to 34 isobutylene monomeric units per molecule. In certain embodiments, the polyisobutylene comprises an average of 3 to 32 isobutylene monomeric units per molecule. In certain embodiments, the polyisobutylene comprises an average of 3 to 30 isobutylene monomeric units per molecule. In certain embodiments, the polyisobutylene comprises an average of 3 to 28 isobutylene monomeric units per molecule. In certain embodiments, the polyisobutylene comprises an average of 3 to 26 isobutylene monomeric units per molecule. In certain embodiments, the polyisobutylene comprises an average of 3 to 24 isobutylene monomeric units per molecule. In certain embodiments, the polyisobutylene comprises an average of 3 to 22 isobutylene monomeric units per molecule. In certain embodiments, the polyisobutylene comprises an average of 3 to 20 isobutylene monomeric units per molecule. In certain embodiments, the polyisobutylene comprises an average of 3 to 18 isobutylene monomeric units per molecule. In certain embodiments, the polyisobutylene comprises an average of 3 to 16 isobutylene monomeric units per molecule. In certain embodiments, the polyisobutylene comprises an average of 3 to 14 isobutylene monomeric units per molecule. In certain embodiments, the polyisobutylene comprises an average of 3 to 12 isobutylene monomeric units per molecule. In certain embodiments, the polyisobutylene comprises an average of 3 to 10 isobutylene monomeric units per molecule. In certain embodiments, the polyisobutylene comprises an average of 3 to 9 isobutylene monomeric units per molecule. In certain embodiments, the polyisobutylene comprises an average of 3 to 8 isobutylene monomeric units per molecule. In certain embodiments, the polyisobutylene comprises an average of 3 to 7 isobutylene monomeric units per molecule. In certain embodiments, the polyisobutylene comprises an average of 3 to 6 isobutylene monomeric units per molecule. In certain embodiments, the polyisobutylene comprises an average of 3 to 5 isobutylene monomeric units per molecule. In certain embodiments, the polyisobutylene comprises an average of 3 to 4 isobutylene monomeric units per molecule. In certain embodiments, the polyisobutylene comprises an average of 4 to 56 isobutylene monomeric units per molecule. In certain embodiments, the polyisobutylene comprises an average of 4 to 55 isobutylene monomeric units per molecule. In certain embodiments, the polyisobutylene comprises an average of 4 to 54 isobutylene monomeric units per molecule. In certain embodiments, the polyisobutylene comprises an average of 4 to 53 isobutylene monomeric units per molecule. In certain embodiments, the polyisobutylene comprises an average of 4 to 52 isobutylene monomeric units per molecule. In certain embodiments, the polyisobutylene comprises an average of 4 to 51 isobutylene monomeric units per molecule. In certain embodiments, the polyisobutylene comprises an average of 4 to 50 isobutylene monomeric units per molecule. In certain embodiments, the polyisobutylene comprises an average of 4 to 48 isobutylene monomeric units per molecule. In certain embodiments, the polyisobutylene comprises an average of 4 to 46 isobutylene monomeric units per molecule. In certain embodiments, the polyisobutylene comprises an average of 4 to 44 isobutylene monomeric units per molecule. In certain embodiments, the polyisobutylene comprises an average of 4 to 42 isobutylene monomeric units per molecule. In certain embodiments, the polyisobutylene comprises an average of 4 to 40 isobutylene monomeric units per molecule. In certain embodiments, the polyisobutylene comprises an average of 4 to 38 isobutylene monomeric units per molecule. In certain embodiments, the polyisobutylene comprises an average of 4 to 36 isobutylene monomeric units per molecule. In certain embodiments, the polyisobutylene comprises an average of 4 to 34 isobutylene monomeric units per molecule. In certain embodiments, the polyisobutylene comprises an average of 4 to 32 isobutylene monomeric units per molecule. In certain embodiments, the polyisobutylene comprises an average of 4 to 30 isobutylene monomeric units per molecule. In certain embodiments, the polyisobutylene comprises an average of 4 to 28 isobutylene monomeric units per molecule. In certain embodiments, the polyisobutylene comprises an average of 4 to 26 isobutylene monomeric units per molecule. In certain embodiments, the polyisobutylene comprises an average of 4 to 24 isobutylene monomeric units per molecule. In certain embodiments, the polyisobutylene comprises an average of 4 to 22 isobutylene monomeric units per molecule. In certain embodiments, the polyisobutylene comprises an average of 4 to 20 isobutylene monomeric units per molecule. In certain embodiments, the polyisobutylene comprises an average of 4 to 18 isobutylene monomeric units per molecule. In certain embodiments, the polyisobutylene comprises an average of 4 to 16 isobutylene monomeric units per molecule. In certain embodiments, the polyisobutylene comprises an average of 4 to 14 isobutylene monomeric units per molecule. In certain embodiments, the polyisobutylene comprises an average of 4 to 12 isobutylene monomeric units per molecule. In certain embodiments, the polyisobutylene comprises an average of 4 to 10 isobutylene monomeric units per molecule. In certain embodiments, the polyisobutylene comprises an average of 4 to 9 isobutylene monomeric units per molecule. In certain embodiments, the polyisobutylene comprises an average of 4 to 8 isobutylene monomeric units per molecule. In certain embodiments, the polyisobutylene comprises an average of 4 to 7 isobutylene monomeric units per molecule. In certain embodiments, the polyisobutylene comprises an average of 4 to 6 isobutylene monomeric units per molecule. In certain embodiments, the polyisobutylene comprises an average of 4 to 5 isobutylene monomeric units per molecule. In certain embodiments, the polyisobutylene comprises an average of 5 to 56 isobutylene monomeric units per molecule. In certain embodiments, the polyisobutylene comprises an average of 5 to 55 isobutylene monomeric units per molecule. In certain embodiments, the polyisobutylene comprises an average of 5 to 54 isobutylene monomeric units per molecule. In certain embodiments, the polyisobutylene comprises an average of 5 to 53 isobutylene monomeric units per molecule. In certain embodiments, the polyisobutylene comprises an average of 5 to 52 isobutylene monomeric units per molecule. In certain embodiments, the polyisobutylene comprises an average of 5 to 51 isobutylene monomeric units per molecule. In certain embodiments, the polyisobutylene comprises an average of 5 to 50 isobutylene monomeric units per molecule. In certain embodiments, the polyisobutylene comprises an average of 5 to 48 isobutylene monomeric units per molecule. In certain embodiments, the polyisobutylene comprises an average of 5 to 46 isobutylene monomeric units per molecule. In certain embodiments, the polyisobutylene comprises an average of 5 to 44 isobutylene monomeric units per molecule. In certain embodiments, the polyisobutylene comprises an average of 5 to 42 isobutylene monomeric units per molecule. In certain embodiments, the polyisobutylene comprises an average of 5 to 40 isobutylene monomeric units per molecule. In certain embodiments, the polyisobutylene comprises an average of 5 to 38 isobutylene monomeric units per molecule. In certain embodiments, the polyisobutylene comprises an average of 5 to 36 isobutylene monomeric units per molecule. In certain embodiments, the polyisobutylene comprises an average of 5 to 34 isobutylene monomeric units per molecule. In certain embodiments, the polyisobutylene comprises an average of 5 to 32 isobutylene monomeric units per molecule. In certain embodiments, the polyisobutylene comprises an average of 5 to 30 isobutylene monomeric units per molecule. In certain embodiments, the polyisobutylene comprises an average of 5 to 28 isobutylene monomeric units per molecule. In certain embodiments, the polyisobutylene comprises an average of 5 to 26 isobutylene monomeric units per molecule. In certain embodiments, the polyisobutylene comprises an average of 5 to 24 isobutylene monomeric units per molecule. In certain embodiments, the polyisobutylene comprises an average of 5 to 22 isobutylene monomeric units per molecule. In certain embodiments, the polyisobutylene comprises an average of 5 to 20 isobutylene monomeric units per molecule. In certain embodiments, the polyisobutylene comprises an average of 5 to 18 isobutylene monomeric units per molecule. In certain embodiments, the polyisobutylene comprises an average of 5 to 16 isobutylene monomeric units per molecule. In certain embodiments, the polyisobutylene comprises an average of 5 to 14 isobutylene monomeric units per molecule. In certain embodiments, the polyisobutylene comprises an average of 5 to 12 isobutylene monomeric units per molecule. In certain embodiments, the polyisobutylene comprises an average of 5 to 10 isobutylene monomeric units per molecule. In certain embodiments, the polyisobutylene comprises an average of 5 to 9 isobutylene monomeric units per molecule. In certain embodiments, the polyisobutylene comprises an average of 5 to 8 isobutylene monomeric units per molecule. In certain embodiments, the polyisobutylene comprises an average of 5 to 7 isobutylene monomeric units per molecule. In certain embodiments, the polyisobutylene comprises an average of 5 to 6 isobutylene monomeric units per molecule. In certain embodiments, the polyisobutylene comprises an average of 6 to 56 isobutylene monomeric units per molecule. In certain embodiments, the polyisobutylene comprises an average of 6 to 55 isobutylene monomeric units per molecule. In certain embodiments, the polyisobutylene comprises an average of 6 to 54 isobutylene monomeric units per molecule. In certain embodiments, the polyisobutylene comprises an average of 6 to 53 isobutylene monomeric units per molecule. In certain embodiments, the polyisobutylene comprises an average of 6 to 52 isobutylene monomeric units per molecule. In certain embodiments, the polyisobutylene comprises an average of 6 to 51 isobutylene monomeric units per molecule. In certain embodiments, the polyisobutylene comprises an average of 6 to 50 isobutylene monomeric units per molecule. In certain embodiments, the polyisobutylene comprises an average of 6 to 48 isobutylene monomeric units per molecule. In certain embodiments, the polyisobutylene comprises an average of 6 to 46 isobutylene monomeric units per molecule. In certain embodiments, the polyisobutylene comprises an average of 6 to 44 isobutylene monomeric units per molecule. In certain embodiments, the polyisobutylene comprises an average of 6 to 42 isobutylene monomeric units per molecule. In certain embodiments, the polyisobutylene comprises an average of 6 to 40 isobutylene monomeric units per molecule. In certain embodiments, the polyisobutylene comprises an average of 6 to 38 isobutylene monomeric units per molecule. In certain embodiments, the polyisobutylene comprises an average of 6 to 36 isobutylene monomeric units per molecule. In certain embodiments, the polyisobutylene comprises an average of 6 to 34 isobutylene monomeric units per molecule. In certain embodiments, the polyisobutylene comprises an average of 6 to 32 isobutylene monomeric units per molecule. In certain embodiments, the polyisobutylene comprises an average of 6 to 30 isobutylene monomeric units per molecule. In certain embodiments, the polyisobutylene comprises an average of 6 to 28 isobutylene monomeric units per molecule. In certain embodiments, the polyisobutylene comprises an average of 6 to 26 isobutylene monomeric units per molecule. In certain embodiments, the polyisobutylene comprises an average of 6 to 24 isobutylene monomeric units per molecule. In certain embodiments, the polyisobutylene comprises an average of 6 to 22 isobutylene monomeric units per molecule. In certain embodiments, the polyisobutylene comprises an average of 6 to 20 isobutylene monomeric units per molecule. In certain embodiments, the polyisobutylene comprises an average of 6 to 18 isobutylene monomeric units per molecule. In certain embodiments, the polyisobutylene comprises an average of 6 to 16 isobutylene monomeric units per molecule. In certain embodiments, the polyisobutylene comprises an average of 6 to 14 isobutylene monomeric units per molecule. In certain embodiments, the polyisobutylene comprises an average of 6 to 12 isobutylene monomeric units per molecule. In certain embodiments, the polyisobutylene comprises an average of 6 to 10 isobutylene monomeric units per molecule. In certain embodiments, the polyisobutylene comprises an average of 6 to 9 isobutylene monomeric units per molecule. In certain embodiments, the polyisobutylene comprises an average of 6 to 8 isobutylene monomeric units per molecule. In certain embodiments, the polyisobutylene comprises an average of 6 to 7 isobutylene monomeric units per molecule. In certain embodiments, the polyisobutylene comprises an average of 7 to 56 isobutylene monomeric units per molecule. In certain embodiments, the polyisobutylene comprises an average of 7 to 55 isobutylene monomeric units per molecule. In certain embodiments, the polyisobutylene comprises an average of 7 to 54 isobutylene monomeric units per molecule. In certain embodiments, the polyisobutylene comprises an average of 7 to 53 isobutylene monomeric units per molecule. In certain embodiments, the polyisobutylene comprises an average of 7 to 52 isobutylene monomeric units per molecule. In certain embodiments, the polyisobutylene comprises an average of 7 to 51 isobutylene monomeric units per molecule. In certain embodiments, the polyisobutylene comprises an average of 7 to 50 isobutylene monomeric units per molecule. In certain embodiments, the polyisobutylene comprises an average of 7 to 48 isobutylene monomeric units per molecule. In certain embodiments, the polyisobutylene comprises an average of 7 to 46 isobutylene monomeric units per molecule. In certain embodiments, the polyisobutylene comprises an average of 7 to 44 isobutylene monomeric units per molecule. In certain embodiments, the polyisobutylene comprises an average of 7 to 42 isobutylene monomeric units per molecule. In certain embodiments, the polyisobutylene comprises an average of 7 to 40 isobutylene monomeric units per molecule. In certain embodiments, the polyisobutylene comprises an average of 7 to 38 isobutylene monomeric units per molecule. In certain embodiments, the polyisobutylene comprises an average of 7 to 36 isobutylene monomeric units per molecule. In certain embodiments, the polyisobutylene comprises an average of 7 to 34 isobutylene monomeric units per molecule. In certain embodiments, the polyisobutylene comprises an average of 7 to 32 isobutylene monomeric units per molecule. In certain embodiments, the polyisobutylene comprises an average of 7 to 30 isobutylene monomeric units per molecule. In certain embodiments, the polyisobutylene comprises an average of 7 to 28 isobutylene monomeric units per molecule. In certain embodiments, the polyisobutylene comprises an average of 7 to 26 isobutylene monomeric units per molecule. In certain embodiments, the polyisobutylene comprises an average of 7 to 24 isobutylene monomeric units per molecule. In certain embodiments, the polyisobutylene comprises an average of 7 to 22 isobutylene monomeric units per molecule. In certain embodiments, the polyisobutylene comprises an average of 7 to 20 isobutylene monomeric units per molecule. In certain embodiments, the polyisobutylene comprises an average of 7 to 18 isobutylene monomeric units per molecule. In certain embodiments, the polyisobutylene comprises an average of 7 to 16 isobutylene monomeric units per molecule. In certain embodiments, the polyisobutylene comprises an average of 7 to 14 isobutylene monomeric units per molecule. In certain embodiments, the polyisobutylene comprises an average of 7 to 12 isobutylene monomeric units per molecule. In certain embodiments, the polyisobutylene comprises an average of 7 to 10 isobutylene monomeric units per molecule. In certain embodiments, the polyisobutylene comprises an average of 7 to 9 isobutylene monomeric units per molecule. In certain embodiments, the polyisobutylene comprises an average of 7 to 8 isobutylene monomeric units per molecule. In certain embodiments, the polyisobutylene comprises an average of 8 to 56 isobutylene monomeric units per molecule. In certain embodiments, the polyisobutylene comprises an average of 8 to 55 isobutylene monomeric units per molecule. In certain embodiments, the polyisobutylene comprises an average of 8 to 54 isobutylene monomeric units per molecule. In certain embodiments, the polyisobutylene comprises an average of 8 to 53 isobutylene monomeric units per molecule. In certain embodiments, the polyisobutylene comprises an average of 8 to 52 isobutylene monomeric units per molecule. In certain embodiments, the polyisobutylene comprises an average of 8 to 51 isobutylene monomeric units per molecule. In certain embodiments, the polyisobutylene comprises an average of 8 to 50 isobutylene monomeric units per molecule. In certain embodiments, the polyisobutylene comprises an average of 8 to 48 isobutylene monomeric units per molecule. In certain embodiments, the polyisobutylene comprises an average of 8 to 46 isobutylene monomeric units per molecule. In certain embodiments, the polyisobutylene comprises an average of 8 to 44 isobutylene monomeric units per molecule. In certain embodiments, the polyisobutylene comprises an average of 8 to 42 isobutylene monomeric units per molecule. In certain embodiments, the polyisobutylene comprises an average of 8 to 40 isobutylene monomeric units per molecule. In certain embodiments, the polyisobutylene comprises an average of 8 to 38 isobutylene monomeric units per molecule. In certain embodiments, the polyisobutylene comprises an average of 8 to 36 isobutylene monomeric units per molecule. In certain embodiments, the polyisobutylene comprises an average of 8 to 34 isobutylene monomeric units per molecule. In certain embodiments, the polyisobutylene comprises an average of 8 to 32 isobutylene monomeric units per molecule. In certain embodiments, the polyisobutylene comprises an average of 8 to 30 isobutylene monomeric units per molecule. In certain embodiments, the polyisobutylene comprises an average of 8 to 28 isobutylene monomeric units per molecule. In certain embodiments, the polyisobutylene comprises an average of 8 to 26 isobutylene monomeric units per molecule. In certain embodiments, the polyisobutylene comprises an average of 8 to 24 isobutylene monomeric units per molecule. In certain embodiments, the polyisobutylene comprises an average of 8 to 22 isobutylene monomeric units per molecule. In certain embodiments, the polyisobutylene comprises an average of 8 to 20 isobutylene monomeric units per molecule. In certain embodiments, the polyisobutylene comprises an average of 8 to 18 isobutylene monomeric units per molecule. In certain embodiments, the polyisobutylene comprises an average of 8 to 16 isobutylene monomeric units per molecule. In certain embodiments, the polyisobutylene comprises an average of 8 to 14 isobutylene monomeric units per molecule. In certain embodiments, the polyisobutylene comprises an average of 8 to 12 isobutylene monomeric units per molecule. In certain embodiments, the polyisobutylene comprises an average of 8 to 10 isobutylene monomeric units per molecule. In certain embodiments, the polyisobutylene comprises an average of 8 to 9 isobutylene monomeric units per molecule. In certain embodiments, the polyisobutylene comprises an average of 9 to 56 isobutylene monomeric units per molecule. In certain embodiments, the polyisobutylene comprises an average of 9 to 55 isobutylene monomeric units per molecule. In certain embodiments, the polyisobutylene comprises an average of 9 to 54 isobutylene monomeric units per molecule. In certain embodiments, the polyisobutylene comprises an average of 9 to 53 isobutylene monomeric units per molecule. In certain embodiments, the polyisobutylene comprises an average of 9 to 52 isobutylene monomeric units per molecule. In certain embodiments, the polyisobutylene comprises an average of 9 to 51 isobutylene monomeric units per molecule. In certain embodiments, the polyisobutylene comprises an average of 9 to 50 isobutylene monomeric units per molecule. In certain embodiments, the polyisobutylene comprises an average of 9 to 48 isobutylene monomeric units per molecule. In certain embodiments, the polyisobutylene comprises an average of 9 to 46 isobutylene monomeric units per molecule. In certain embodiments, the polyisobutylene comprises an average of 9 to 44 isobutylene monomeric units per molecule. In certain embodiments, the polyisobutylene comprises an average of 9 to 42 isobutylene monomeric units per molecule. In certain embodiments, the polyisobutylene comprises an average of 9 to 40 isobutylene monomeric units per molecule. In certain embodiments, the polyisobutylene comprises an average of 9 to 38 isobutylene monomeric units per molecule. In certain embodiments, the polyisobutylene comprises an average of 9 to 36 isobutylene monomeric units per molecule. In certain embodiments, the polyisobutylene comprises an average of 9 to 34 isobutylene monomeric units per molecule. In certain embodiments, the polyisobutylene comprises an average of 9 to 32 isobutylene monomeric units per molecule. In certain embodiments, the polyisobutylene comprises an average of 9 to 30 isobutylene monomeric units per molecule. In certain embodiments, the polyisobutylene comprises an average of 9 to 28 isobutylene monomeric units per molecule. In certain embodiments, the polyisobutylene comprises an average of 9 to 26 isobutylene monomeric units per molecule. In certain embodiments, the polyisobutylene comprises an average of 9 to 24 isobutylene monomeric units per molecule. In certain embodiments, the polyisobutylene comprises an average of 9 to 22 isobutylene monomeric units per molecule. In certain embodiments, the polyisobutylene comprises an average of 9 to 20 isobutylene monomeric units per molecule. In certain embodiments, the polyisobutylene comprises an average of 9 to 18 isobutylene monomeric units per molecule. In certain embodiments, the polyisobutylene comprises an average of 9 to 16 isobutylene monomeric units per molecule. In certain embodiments, the polyisobutylene comprises an average of 9 to 14 isobutylene monomeric units per molecule. In certain embodiments, the polyisobutylene comprises an average of 9 to 12 isobutylene monomeric units per molecule. In certain embodiments, the polyisobutylene comprises an average of 9 to 10 isobutylene monomeric units per molecule. In certain embodiments, the polyisobutylene comprises an average of 10 to 56 isobutylene monomeric units per molecule. In certain embodiments, the polyisobutylene comprises an average of 10 to 55 isobutylene monomeric units per molecule. In certain embodiments, the polyisobutylene comprises an average of 10 to 54 isobutylene monomeric units per molecule. In certain embodiments, the polyisobutylene comprises an average of 10 to 53 isobutylene monomeric units per molecule. In certain embodiments, the polyisobutylene comprises an average of 10 to 52 isobutylene monomeric units per molecule. In certain embodiments, the polyisobutylene comprises an average of 10 to 51 isobutylene monomeric units per molecule. In certain embodiments, the polyisobutylene comprises an average of 10 to 50 isobutylene monomeric units per molecule. In certain embodiments, the polyisobutylene comprises an average of 10 to 48 isobutylene monomeric units per molecule. In certain embodiments, the polyisobutylene comprises an average of 10 to 46 isobutylene monomeric units per molecule. In certain embodiments, the polyisobutylene comprises an average of 10 to 44 isobutylene monomeric units per molecule. In certain embodiments, the polyisobutylene comprises an average of 10 to 42 isobutylene monomeric units per molecule. In certain embodiments, the polyisobutylene comprises an average of 10 to 40 isobutylene monomeric units per molecule. In certain embodiments, the polyisobutylene comprises an average of 10 to 38 isobutylene monomeric units per molecule. In certain embodiments, the polyisobutylene comprises an average of 10 to 36 isobutylene monomeric units per molecule. In certain embodiments, the polyisobutylene comprises an average of 10 to 34 isobutylene monomeric units per molecule. In certain embodiments, the polyisobutylene comprises an average of 10 to 32 isobutylene monomeric units per molecule. In certain embodiments, the polyisobutylene comprises an average of 10 to 30 isobutylene monomeric units per molecule. In certain embodiments, the polyisobutylene comprises an average of 10 to 28 isobutylene monomeric units per molecule. In certain embodiments, the polyisobutylene comprises an average of 10 to 26 isobutylene monomeric units per molecule. In certain embodiments, the polyisobutylene comprises an average of 10 to 24 isobutylene monomeric units per molecule. In certain embodiments, the polyisobutylene comprises an average of 10 to 22 isobutylene monomeric units per molecule. In certain embodiments, the polyisobutylene comprises an average of 10 to 20 isobutylene monomeric units per molecule. In certain embodiments, the polyisobutylene comprises an average of 10 to 18 isobutylene monomeric units per molecule. In certain embodiments, the polyisobutylene comprises an average of 10 to 16 isobutylene monomeric units per molecule. In certain embodiments, the polyisobutylene comprises an average of 10 to 14 isobutylene monomeric units per molecule. In certain embodiments, the polyisobutylene comprises an average of 10 to 12 isobutylene monomeric units per molecule. In certain embodiments, the polyisobutylene comprises an average of 12 to 56 isobutylene monomeric units per molecule. In certain embodiments, the polyisobutylene comprises an average of 12 to 55 isobutylene monomeric units per molecule. In certain embodiments, the polyisobutylene comprises an average of 12 to 54 isobutylene monomeric units per molecule. In certain embodiments, the polyisobutylene comprises an average of 12 to 53 isobutylene monomeric units per molecule. In certain embodiments, the polyisobutylene comprises an average of 12 to 52 isobutylene monomeric units per molecule. In certain embodiments, the polyisobutylene comprises an average of 12 to 51 isobutylene monomeric units per molecule. In certain embodiments, the polyisobutylene comprises an average of 12 to 50 isobutylene monomeric units per molecule. In certain embodiments, the polyisobutylene comprises an average of 12 to 48 isobutylene monomeric units per molecule. In certain embodiments, the polyisobutylene comprises an average of 12 to 46 isobutylene monomeric units per molecule. In certain embodiments, the polyisobutylene comprises an average of 12 to 44 isobutylene monomeric units per molecule. In certain embodiments, the polyisobutylene comprises an average of 12 to 42 isobutylene monomeric units per molecule. In certain embodiments, the polyisobutylene comprises an average of 12 to 40 isobutylene monomeric units per molecule. In certain embodiments, the polyisobutylene comprises an average of 12 to 38 isobutylene monomeric units per molecule. In certain embodiments, the polyisobutylene comprises an average of 12 to 36 isobutylene monomeric units per molecule. In certain embodiments, the polyisobutylene comprises an average of 12 to 34 isobutylene monomeric units per molecule. In certain embodiments, the polyisobutylene comprises an average of 12 to 32 isobutylene monomeric units per molecule. In certain embodiments, the polyisobutylene comprises an average of 12 to 30 isobutylene monomeric units per molecule. In certain embodiments, the polyisobutylene comprises an average of 12 to 28 isobutylene monomeric units per molecule. In certain embodiments, the polyisobutylene comprises an average of 12 to 26 isobutylene monomeric units per molecule. In certain embodiments, the polyisobutylene comprises an average of 12 to 24 isobutylene monomeric units per molecule. In certain embodiments, the polyisobutylene comprises an average of 12 to 22 isobutylene monomeric units per molecule. In certain embodiments, the polyisobutylene comprises an average of 12 to 20 isobutylene monomeric units per molecule. In certain embodiments, the polyisobutylene comprises an average of 12 to 18 isobutylene monomeric units per molecule. In certain embodiments, the polyisobutylene comprises an average of 12 to 16 isobutylene monomeric units per molecule. In certain embodiments, the polyisobutylene comprises an average of 12 to 14 isobutylene monomeric units per molecule. In certain embodiments, the polyisobutylene comprises an average of 14 to 56 isobutylene monomeric units per molecule. In certain embodiments, the polyisobutylene comprises an average of 14 to 55 isobutylene monomeric units per molecule. In certain embodiments, the polyisobutylene comprises an average of 14 to 54 isobutylene monomeric units per molecule. In certain embodiments, the polyisobutylene comprises an average of 14 to 53 isobutylene monomeric units per molecule. In certain embodiments, the polyisobutylene comprises an average of 14 to 52 isobutylene monomeric units per molecule. In certain embodiments, the polyisobutylene comprises an average of 14 to 51 isobutylene monomeric units per molecule. In certain embodiments, the polyisobutylene comprises an average of 14 to 50 isobutylene monomeric units per molecule. In certain embodiments, the polyisobutylene comprises an average of 14 to 48 isobutylene monomeric units per molecule. In certain embodiments, the polyisobutylene comprises an average of 14 to 46 isobutylene monomeric units per molecule. In certain embodiments, the polyisobutylene comprises an average of 14 to 44 isobutylene monomeric units per molecule. In certain embodiments, the polyisobutylene comprises an average of 14 to 42 isobutylene monomeric units per molecule. In certain embodiments, the polyisobutylene comprises an average of 14 to 40 isobutylene monomeric units per molecule. In certain embodiments, the polyisobutylene comprises an average of 14 to 38 isobutylene monomeric units per molecule. In certain embodiments, the polyisobutylene comprises an average of 14 to 36 isobutylene monomeric units per molecule. In certain embodiments, the polyisobutylene comprises an average of 14 to 34 isobutylene monomeric units per molecule. In certain embodiments, the polyisobutylene comprises an average of 14 to 32 isobutylene monomeric units per molecule. In certain embodiments, the polyisobutylene comprises an average of 14 to 30 isobutylene monomeric units per molecule. In certain embodiments, the polyisobutylene comprises an average of 14 to 28 isobutylene monomeric units per molecule. In certain embodiments, the polyisobutylene comprises an average of 14 to 26 isobutylene monomeric units per molecule. In certain embodiments, the polyisobutylene comprises an average of 14 to 24 isobutylene monomeric units per molecule. In certain embodiments, the polyisobutylene comprises an average of 14 to 22 isobutylene monomeric units per molecule. In certain embodiments, the polyisobutylene comprises an average of 14 to 20 isobutylene monomeric units per molecule. In certain embodiments, the polyisobutylene comprises an average of 14 to 18 isobutylene monomeric units per molecule. In certain embodiments, the polyisobutylene comprises an average of 14 to 16 isobutylene monomeric units per molecule. In certain embodiments, the polyisobutylene comprises an average of 16 to 56 isobutylene monomeric units per molecule. In certain embodiments, the polyisobutylene comprises an average of 16 to 55 isobutylene monomeric units per molecule. In certain embodiments, the polyisobutylene comprises an average of 16 to 54 isobutylene monomeric units per molecule. In certain embodiments, the polyisobutylene comprises an average of 16 to 53 isobutylene monomeric units per molecule. In certain embodiments, the polyisobutylene comprises an average of 16 to 52 isobutylene monomeric units per molecule. In certain embodiments, the polyisobutylene comprises an average of 16 to 51 isobutylene monomeric units per molecule. In certain embodiments, the polyisobutylene comprises an average of 16 to 50 isobutylene monomeric units per molecule. In certain embodiments, the polyisobutylene comprises an average of 16 to 48 isobutylene monomeric units per molecule. In certain embodiments, the polyisobutylene comprises an average of 16 to 46 isobutylene monomeric units per molecule. In certain embodiments, the polyisobutylene comprises an average of 16 to 44 isobutylene monomeric units per molecule. In certain embodiments, the polyisobutylene comprises an average of 16 to 42 isobutylene monomeric units per molecule. In certain embodiments, the polyisobutylene comprises an average of 16 to 40 isobutylene monomeric units per molecule. In certain embodiments, the polyisobutylene comprises an average of 16 to 38 isobutylene monomeric units per molecule. In certain embodiments, the polyisobutylene comprises an average of 16 to 36 isobutylene monomeric units per molecule. In certain embodiments, the polyisobutylene comprises an average of 16 to 34 isobutylene monomeric units per molecule. In certain embodiments, the polyisobutylene comprises an average of 16 to 32 isobutylene monomeric units per molecule. In certain embodiments, the polyisobutylene comprises an average of 16 to 30 isobutylene monomeric units per molecule. In certain embodiments, the polyisobutylene comprises an average of 16 to 28 isobutylene monomeric units per molecule. In certain embodiments, the polyisobutylene comprises an average of 16 to 26 isobutylene monomeric units per molecule. In certain embodiments, the polyisobutylene comprises an average of 16 to 24 isobutylene monomeric units per molecule. In certain embodiments, the polyisobutylene comprises an average of 16 to 22 isobutylene monomeric units per molecule. In certain embodiments, the polyisobutylene comprises an average of 16 to 20 isobutylene monomeric units per molecule. In certain embodiments, the polyisobutylene comprises an average of 16 to 18 isobutylene monomeric units per molecule. In certain embodiments, the polyisobutylene comprises an average of 18 to 56 isobutylene monomeric units per molecule. In certain embodiments, the polyisobutylene comprises an average of 18 to 55 isobutylene monomeric units per molecule. In certain embodiments, the polyisobutylene comprises an average of 18 to 54 isobutylene monomeric units per molecule. In certain embodiments, the polyisobutylene comprises an average of 18 to 53 isobutylene monomeric units per molecule. In certain embodiments, the polyisobutylene comprises an average of 18 to 52 isobutylene monomeric units per molecule. In certain embodiments, the polyisobutylene comprises an average of 18 to 51 isobutylene monomeric units per molecule. In certain embodiments, the polyisobutylene comprises an average of 18 to 50 isobutylene monomeric units per molecule. In certain embodiments, the polyisobutylene comprises an average of 18 to 48 isobutylene monomeric units per molecule. In certain embodiments, the polyisobutylene comprises an average of 18 to 46 isobutylene monomeric units per molecule. In certain embodiments, the polyisobutylene comprises an average of 18 to 44 isobutylene monomeric units per molecule. In certain embodiments, the polyisobutylene comprises an average of 18 to 42 isobutylene monomeric units per molecule. In certain embodiments, the polyisobutylene comprises an average of 18 to 40 isobutylene monomeric units per molecule. In certain embodiments, the polyisobutylene comprises an average of 18 to 38 isobutylene monomeric units per molecule. In certain embodiments, the polyisobutylene comprises an average of 18 to 36 isobutylene monomeric units per molecule. In certain embodiments, the polyisobutylene comprises an average of 18 to 34 isobutylene monomeric units per molecule. In certain embodiments, the polyisobutylene comprises an average of 18 to 32 isobutylene monomeric units per molecule. In certain embodiments, the polyisobutylene comprises an average of 18 to 30 isobutylene monomeric units per molecule. In certain embodiments, the polyisobutylene comprises an average of 18 to 28 isobutylene monomeric units per molecule. In certain embodiments, the polyisobutylene comprises an average of 18 to 26 isobutylene monomeric units per molecule. In certain embodiments, the polyisobutylene comprises an average of 18 to 24 isobutylene monomeric units per molecule. In certain embodiments, the polyisobutylene comprises an average of 18 to 22 isobutylene monomeric units per molecule. In certain embodiments, the polyisobutylene comprises an average of 18 to 20 isobutylene monomeric units per molecule. In certain embodiments, the polyisobutylene comprises an average of 20 to 56 isobutylene monomeric units per molecule. In certain embodiments, the polyisobutylene comprises an average of 20 to 55 isobutylene monomeric units per molecule. In certain embodiments, the polyisobutylene comprises an average of 20 to 54 isobutylene monomeric units per molecule. In certain embodiments, the polyisobutylene comprises an average of 20 to 53 isobutylene monomeric units per molecule. In certain embodiments, the polyisobutylene comprises an average of 20 to 52 isobutylene monomeric units per molecule. In certain embodiments, the polyisobutylene comprises an average of 20 to 51 isobutylene monomeric units per molecule. In certain embodiments, the polyisobutylene comprises an average of 20 to 50 isobutylene monomeric units per molecule. In certain embodiments, the polyisobutylene comprises an average of 20 to 48 isobutylene monomeric units per molecule. In certain embodiments, the polyisobutylene comprises an average of 20 to 46 isobutylene monomeric units per molecule. In certain embodiments, the polyisobutylene comprises an average of 20 to 44 isobutylene monomeric units per molecule. In certain embodiments, the polyisobutylene comprises an average of 20 to 42 isobutylene monomeric units per molecule. In certain embodiments, the polyisobutylene comprises an average of 20 to 40 isobutylene monomeric units per molecule. In certain embodiments, the polyisobutylene comprises an average of 20 to 38 isobutylene monomeric units per molecule. In certain embodiments, the polyisobutylene comprises an average of 20 to 36 isobutylene monomeric units per molecule. In certain embodiments, the polyisobutylene comprises an average of 20 to 34 isobutylene monomeric units per molecule. In certain embodiments, the polyisobutylene comprises an average of 20 to 32 isobutylene monomeric units per molecule. In certain embodiments, the polyisobutylene comprises an average of 20 to 30 isobutylene monomeric units per molecule. In certain embodiments, the polyisobutylene comprises an average of 20 to 28 isobutylene monomeric units per molecule. In certain embodiments, the polyisobutylene comprises an average of 20 to 26 isobutylene monomeric units per molecule. In certain embodiments, the polyisobutylene comprises an average of 20 to 24 isobutylene monomeric units per molecule. In certain embodiments, the polyisobutylene comprises an average of 20 to 22 isobutylene monomeric units per molecule. In certain embodiments, the polyisobutylene comprises an average of 22 to 56 isobutylene monomeric units per molecule. In certain embodiments, the polyisobutylene comprises an average of 22 to 55 isobutylene monomeric units per molecule. In certain embodiments, the polyisobutylene comprises an average of 22 to 54 isobutylene monomeric units per molecule. In certain embodiments, the polyisobutylene comprises an average of 22 to 53 isobutylene monomeric units per molecule. In certain embodiments, the polyisobutylene comprises an average of 22 to 52 isobutylene monomeric units per molecule. In certain embodiments, the polyisobutylene comprises an average of 22 to 51 isobutylene monomeric units per molecule. In certain embodiments, the polyisobutylene comprises an average of 22 to 50 isobutylene monomeric units per molecule. In certain embodiments, the polyisobutylene comprises an average of 22 to 48 isobutylene monomeric units per molecule. In certain embodiments, the polyisobutylene comprises an average of 22 to 46 isobutylene monomeric units per molecule. In certain embodiments, the polyisobutylene comprises an average of 22 to 44 isobutylene monomeric units per molecule. In certain embodiments, the polyisobutylene comprises an average of 22 to 42 isobutylene monomeric units per molecule. In certain embodiments, the polyisobutylene comprises an average of 22 to 40 isobutylene monomeric units per molecule. In certain embodiments, the polyisobutylene comprises an average of 22 to 38 isobutylene monomeric units per molecule. In certain embodiments, the polyisobutylene comprises an average of 22 to 36 isobutylene monomeric units per molecule. In certain embodiments, the polyisobutylene comprises an average of 22 to 34 isobutylene monomeric units per molecule. In certain embodiments, the polyisobutylene comprises an average of 22 to 32 isobutylene monomeric units per molecule. In certain embodiments, the polyisobutylene comprises an average of 22 to 30 isobutylene monomeric units per molecule. In certain embodiments, the polyisobutylene comprises an average of 22 to 28 isobutylene monomeric units per molecule. In certain embodiments, the polyisobutylene comprises an average of 22 to 26 isobutylene monomeric units per molecule. In certain embodiments, the polyisobutylene comprises an average of 22 to 24 isobutylene monomeric units per molecule. In certain embodiments, the polyisobutylene comprises an average of 24 to 56 isobutylene monomeric units per molecule. In certain embodiments, the polyisobutylene comprises an average of 24 to 55 isobutylene monomeric units per molecule. In certain embodiments, the polyisobutylene comprises an average of 24 to 54 isobutylene monomeric units per molecule. In certain embodiments, the polyisobutylene comprises an average of 24 to 53 isobutylene monomeric units per molecule. In certain embodiments, the polyisobutylene comprises an average of 24 to 52 isobutylene monomeric units per molecule. In certain embodiments, the polyisobutylene comprises an average of 24 to 51 isobutylene monomeric units per molecule. In certain embodiments, the polyisobutylene comprises an average of 24 to 50 isobutylene monomeric units per molecule. In certain embodiments, the polyisobutylene comprises an average of 24 to 48 isobutylene monomeric units per molecule. In certain embodiments, the polyisobutylene comprises an average of 24 to 46 isobutylene monomeric units per molecule. In certain embodiments, the polyisobutylene comprises an average of 24 to 44 isobutylene monomeric units per molecule. In certain embodiments, the polyisobutylene comprises an average of 24 to 42 isobutylene monomeric units per molecule. In certain embodiments, the polyisobutylene comprises an average of 24 to 40 isobutylene monomeric units per molecule. In certain embodiments, the polyisobutylene comprises an average of 24 to 38 isobutylene monomeric units per molecule. In certain embodiments, the polyisobutylene comprises an average of 24 to 36 isobutylene monomeric units per molecule. In certain embodiments, the polyisobutylene comprises an average of 24 to 34 isobutylene monomeric units per molecule. In certain embodiments, the polyisobutylene comprises an average of 24 to 32 isobutylene monomeric units per molecule. In certain embodiments, the polyisobutylene comprises an average of 24 to 30 isobutylene monomeric units per molecule. In certain embodiments, the polyisobutylene comprises an average of 24 to 28 isobutylene monomeric units per molecule. In certain embodiments, the polyisobutylene comprises an average of 24 to 26 isobutylene monomeric units per molecule. In certain embodiments, the polyisobutylene comprises an average of 26 to 56 isobutylene monomeric units per molecule. In certain embodiments, the polyisobutylene comprises an average of 26 to 55 isobutylene monomeric units per molecule. In certain embodiments, the polyisobutylene comprises an average of 26 to 54 isobutylene monomeric units per molecule. In certain embodiments, the polyisobutylene comprises an average of 26 to 53 isobutylene monomeric units per molecule. In certain embodiments, the polyisobutylene comprises an average of 26 to 52 isobutylene monomeric units per molecule. In certain embodiments, the polyisobutylene comprises an average of 26 to 51 isobutylene monomeric units per molecule. In certain embodiments, the polyisobutylene comprises an average of 26 to 50 isobutylene monomeric units per molecule. In certain embodiments, the polyisobutylene comprises an average of 26 to 48 isobutylene monomeric units per molecule. In certain embodiments, the polyisobutylene comprises an average of 26 to 46 isobutylene monomeric units per molecule. In certain embodiments, the polyisobutylene comprises an average of 26 to 44 isobutylene monomeric units per molecule. In certain embodiments, the polyisobutylene comprises an average of 26 to 42 isobutylene monomeric units per molecule. In certain embodiments, the polyisobutylene comprises an average of 26 to 40 isobutylene monomeric units per molecule. In certain embodiments, the polyisobutylene comprises an average of 26 to 38 isobutylene monomeric units per molecule. In certain embodiments, the polyisobutylene comprises an average of 26 to 36 isobutylene monomeric units per molecule. In certain embodiments, the polyisobutylene comprises an average of 26 to 34 isobutylene monomeric units per molecule. In certain embodiments, the polyisobutylene comprises an average of 26 to 32 isobutylene monomeric units per molecule. In certain embodiments, the polyisobutylene comprises an average of 26 to 30 isobutylene monomeric units per molecule. In certain embodiments, the polyisobutylene comprises an average of 26 to 28 isobutylene monomeric units per molecule. In certain embodiments, the polyisobutylene comprises an average of 28 to 56 isobutylene monomeric units per molecule. In certain embodiments, the polyisobutylene comprises an average of 28 to 55 isobutylene monomeric units per molecule. In certain embodiments, the polyisobutylene comprises an average of 28 to 54 isobutylene monomeric units per molecule. In certain embodiments, the polyisobutylene comprises an average of 28 to 53 isobutylene monomeric units per molecule. In certain embodiments, the polyisobutylene comprises an average of 28 to 52 isobutylene monomeric units per molecule. In certain embodiments, the polyisobutylene comprises an average of 28 to 51 isobutylene monomeric units per molecule. In certain embodiments, the polyisobutylene comprises an average of 28 to 50 isobutylene monomeric units per molecule. In certain embodiments, the polyisobutylene comprises an average of 28 to 48 isobutylene monomeric units per molecule. In certain embodiments, the polyisobutylene comprises an average of 28 to 46 isobutylene monomeric units per molecule. In certain embodiments, the polyisobutylene comprises an average of 28 to 44 isobutylene monomeric units per molecule. In certain embodiments, the polyisobutylene comprises an average of 28 to 42 isobutylene monomeric units per molecule. In certain embodiments, the polyisobutylene comprises an average of 28 to 40 isobutylene monomeric units per molecule. In certain embodiments, the polyisobutylene comprises an average of 28 to 38 isobutylene monomeric units per molecule. In certain embodiments, the polyisobutylene comprises an average of 28 to 36 isobutylene monomeric units per molecule. In certain embodiments, the polyisobutylene comprises an average of 28 to 34 isobutylene monomeric units per molecule. In certain embodiments, the polyisobutylene comprises an average of 28 to 32 isobutylene monomeric units per molecule. In certain embodiments, the polyisobutylene comprises an average of 28 to 30 isobutylene monomeric units per molecule. In certain embodiments, the polyisobutylene comprises an average of 30 to 56 isobutylene monomeric units per molecule. In certain embodiments, the polyisobutylene comprises an average of 30 to 55 isobutylene monomeric units per molecule. In certain embodiments, the polyisobutylene comprises an average of 30 to 54 isobutylene monomeric units per molecule. In certain embodiments, the polyisobutylene comprises an average of 30 to 53 isobutylene monomeric units per molecule. In certain embodiments, the polyisobutylene comprises an average of 30 to 52 isobutylene monomeric units per molecule. In certain embodiments, the polyisobutylene comprises an average of 30 to 51 isobutylene monomeric units per molecule. In certain embodiments, the polyisobutylene comprises an average of 30 to 50 isobutylene monomeric units per molecule. In certain embodiments, the polyisobutylene comprises an average of 30 to 48 isobutylene monomeric units per molecule. In certain embodiments, the polyisobutylene comprises an average of 30 to 46 isobutylene monomeric units per molecule. In certain embodiments, the polyisobutylene comprises an average of 30 to 44 isobutylene monomeric units per molecule. In certain embodiments, the polyisobutylene comprises an average of 30 to 42 isobutylene monomeric units per molecule. In certain embodiments, the polyisobutylene comprises an average of 30 to 40 isobutylene monomeric units per molecule. In certain embodiments, the polyisobutylene comprises an average of 30 to 38 isobutylene monomeric units per molecule. In certain embodiments, the polyisobutylene comprises an average of 30 to 36 isobutylene monomeric units per molecule. In certain embodiments, the polyisobutylene comprises an average of 30 to 34 isobutylene monomeric units per molecule. In certain embodiments, the polyisobutylene comprises an average of 30 to 32 isobutylene monomeric units per molecule. In certain embodiments, the polyisobutylene comprises an average of 32 to 56 isobutylene monomeric units per molecule. In certain embodiments, the polyisobutylene comprises an average of 32 to 55 isobutylene monomeric units per molecule. In certain embodiments, the polyisobutylene comprises an average of 32 to 54 isobutylene monomeric units per molecule. In certain embodiments, the polyisobutylene comprises an average of 32 to 53 isobutylene monomeric units per molecule. In certain embodiments, the polyisobutylene comprises an average of 32 to 52 isobutylene monomeric units per molecule. In certain embodiments, the polyisobutylene comprises an average of 32 to 51 isobutylene monomeric units per molecule. In certain embodiments, the polyisobutylene comprises an average of 32 to 50 isobutylene monomeric units per molecule. In certain embodiments, the polyisobutylene comprises an average of 32 to 48 isobutylene monomeric units per molecule. In certain embodiments, the polyisobutylene comprises an average of 32 to 46 isobutylene monomeric units per molecule. In certain embodiments, the polyisobutylene comprises an average of 32 to 44 isobutylene monomeric units per molecule. In certain embodiments, the polyisobutylene comprises an average of 32 to 42 isobutylene monomeric units per molecule. In certain embodiments, the polyisobutylene comprises an average of 32 to 40 isobutylene monomeric units per molecule. In certain embodiments, the polyisobutylene comprises an average of 32 to 38 isobutylene monomeric units per molecule. In certain embodiments, the polyisobutylene comprises an average of 32 to 36 isobutylene monomeric units per molecule. In certain embodiments, the polyisobutylene comprises an average of 32 to 34 isobutylene monomeric units per molecule. In certain embodiments, the polyisobutylene comprises an average of 34 to 56 isobutylene monomeric units per molecule. In certain embodiments, the polyisobutylene comprises an average of 34 to 55 isobutylene monomeric units per molecule. In certain embodiments, the polyisobutylene comprises an average of 34 to 54 isobutylene monomeric units per molecule. In certain embodiments, the polyisobutylene comprises an average of 34 to 53 isobutylene monomeric units per molecule. In certain embodiments, the polyisobutylene comprises an average of 34 to 52 isobutylene monomeric units per molecule. In certain embodiments, the polyisobutylene comprises an average of 34 to 51 isobutylene monomeric units per molecule. In certain embodiments, the polyisobutylene comprises an average of 34 to 50 isobutylene monomeric units per molecule. In certain embodiments, the polyisobutylene comprises an average of 34 to 48 isobutylene monomeric units per molecule. In certain embodiments, the polyisobutylene comprises an average of 34 to 46 isobutylene monomeric units per molecule. In certain embodiments, the polyisobutylene comprises an average of 34 to 44 isobutylene monomeric units per molecule. In certain embodiments, the polyisobutylene comprises an average of 34 to 42 isobutylene monomeric units per molecule. In certain embodiments, the polyisobutylene comprises an average of 34 to 40 isobutylene monomeric units per molecule. In certain embodiments, the polyisobutylene comprises an average of 34 to 38 isobutylene monomeric units per molecule. In certain embodiments, the polyisobutylene comprises an average of 34 to 36 isobutylene monomeric units per molecule. In certain embodiments, the polyisobutylene comprises an average of 36 to 56 isobutylene monomeric units per molecule. In certain embodiments, the polyisobutylene comprises an average of 36 to 55 isobutylene monomeric units per molecule. In certain embodiments, the polyisobutylene comprises an average of 36 to 54 isobutylene monomeric units per molecule. In certain embodiments, the polyisobutylene comprises an average of 36 to 53 isobutylene monomeric units per molecule. In certain embodiments, the polyisobutylene comprises an average of 36 to 52 isobutylene monomeric units per molecule. In certain embodiments, the polyisobutylene comprises an average of 36 to 51 isobutylene monomeric units per molecule. In certain embodiments, the polyisobutylene comprises an average of 36 to 50 isobutylene monomeric units per molecule. In certain embodiments, the polyisobutylene comprises an average of 36 to 48 isobutylene monomeric units per molecule. In certain embodiments, the polyisobutylene comprises an average of 36 to 46 isobutylene monomeric units per molecule. In certain embodiments, the polyisobutylene comprises an average of 36 to 44 isobutylene monomeric units per molecule. In certain embodiments, the polyisobutylene comprises an average of 36 to 42 isobutylene monomeric units per molecule. In certain embodiments, the polyisobutylene comprises an average of 36 to 40 isobutylene monomeric units per molecule. In certain embodiments, the polyisobutylene comprises an average of 36 to 38 isobutylene monomeric units per molecule. In certain embodiments, the polyisobutylene comprises an average of 38 to 56 isobutylene monomeric units per molecule. In certain embodiments, the polyisobutylene comprises an average of 38 to 55 isobutylene monomeric units per molecule. In certain embodiments, the polyisobutylene comprises an average of 38 to 54 isobutylene monomeric units per molecule. In certain embodiments, the polyisobutylene comprises an average of 38 to 53 isobutylene monomeric units per molecule. In certain embodiments, the polyisobutylene comprises an average of 38 to 52 isobutylene monomeric units per molecule. In certain embodiments, the polyisobutylene comprises an average of 38 to 51 isobutylene monomeric units per molecule. In certain embodiments, the polyisobutylene comprises an average of 38 to 50 isobutylene monomeric units per molecule. In certain embodiments, the polyisobutylene comprises an average of 38 to 48 isobutylene monomeric units per molecule. In certain embodiments, the polyisobutylene comprises an average of 38 to 46 isobutylene monomeric units per molecule. In certain embodiments, the polyisobutylene comprises an average of 38 to 44 isobutylene monomeric units per molecule. In certain embodiments, the polyisobutylene comprises an average of 38 to 42 isobutylene monomeric units per molecule. In certain embodiments, the polyisobutylene comprises an average of 38 to 40 isobutylene monomeric units per molecule. In certain embodiments, the polyisobutylene comprises an average of 40 to 56 isobutylene monomeric units per molecule. In certain embodiments, the polyisobutylene comprises an average of 40 to 55 isobutylene monomeric units per molecule. In certain embodiments, the polyisobutylene comprises an average of 40 to 54 isobutylene monomeric units per molecule. In certain embodiments, the polyisobutylene comprises an average of 40 to 53 isobutylene monomeric units per molecule. In certain embodiments, the polyisobutylene comprises an average of 40 to 52 isobutylene monomeric units per molecule. In certain embodiments, the polyisobutylene comprises an average of 40 to 51 isobutylene monomeric units per molecule. In certain embodiments, the polyisobutylene comprises an average of 40 to 50 isobutylene monomeric units per molecule. In certain embodiments, the polyisobutylene comprises an average of 40 to 48 isobutylene monomeric units per molecule. In certain embodiments, the polyisobutylene comprises an average of 40 to 46 isobutylene monomeric units per molecule. In certain embodiments, the polyisobutylene comprises an average of 40 to 44 isobutylene monomeric units per molecule. In certain embodiments, the polyisobutylene comprises an average of 40 to 42 isobutylene monomeric units per molecule. In certain embodiments, the polyisobutylene comprises an average of 42 to 56 isobutylene monomeric units per molecule. In certain embodiments, the polyisobutylene comprises an average of 42 to 55 isobutylene monomeric units per molecule. In certain embodiments, the polyisobutylene comprises an average of 42 to 54 isobutylene monomeric units per molecule. In certain embodiments, the polyisobutylene comprises an average of 42 to 53 isobutylene monomeric units per molecule. In certain embodiments, the polyisobutylene comprises an average of 42 to 52 isobutylene monomeric units per molecule. In certain embodiments, the polyisobutylene comprises an average of 42 to 51 isobutylene monomeric units per molecule. In certain embodiments, the polyisobutylene comprises an average of 42 to 50 isobutylene monomeric units per molecule. In certain embodiments, the polyisobutylene comprises an average of 42 to 48 isobutylene monomeric units per molecule. In certain embodiments, the polyisobutylene comprises an average of 42 to 46 isobutylene monomeric units per molecule. In certain embodiments, the polyisobutylene comprises an average of 42 to 44 isobutylene monomeric units per molecule. In certain embodiments, the polyisobutylene comprises an average of 44 to 56 isobutylene monomeric units per molecule. In certain embodiments, the polyisobutylene comprises an average of 44 to 55 isobutylene monomeric units per molecule. In certain embodiments, the polyisobutylene comprises an average of 44 to 54 isobutylene monomeric units per molecule. In certain embodiments, the polyisobutylene comprises an average of 44 to 53 isobutylene monomeric units per molecule. In certain embodiments, the polyisobutylene comprises an average of 44 to 52 isobutylene monomeric units per molecule. In certain embodiments, the polyisobutylene comprises an average of 44 to 51 isobutylene monomeric units per molecule. In certain embodiments, the polyisobutylene comprises an average of 44 to 50 isobutylene monomeric units per molecule. In certain embodiments, the polyisobutylene comprises an average of 44 to 48 isobutylene monomeric units per molecule. In certain embodiments, the polyisobutylene comprises an average of 44 to 46 isobutylene monomeric units per molecule. In certain embodiments, the polyisobutylene comprises an average of 46 to 56 isobutylene monomeric units per molecule. In certain embodiments, the polyisobutylene comprises an average of 46 to 55 isobutylene monomeric units per molecule. In certain embodiments, the polyisobutylene comprises an average of 46 to 54 isobutylene monomeric units per molecule. In certain embodiments, the polyisobutylene comprises an average of 46 to 53 isobutylene monomeric units per molecule. In certain embodiments, the polyisobutylene comprises an average of 46 to 52 isobutylene monomeric units per molecule. In certain embodiments, the polyisobutylene comprises an average of 46 to 51 isobutylene monomeric units per molecule. In certain embodiments, the polyisobutylene comprises an average of 46 to 50 isobutylene monomeric units per molecule. In certain embodiments, the polyisobutylene comprises an average of 46 to 48 isobutylene monomeric units per molecule. In certain embodiments, the polyisobutylene comprises an average of 48 to 56 isobutylene monomeric units per molecule. In certain embodiments, the polyisobutylene comprises an average of 48 to 55 isobutylene monomeric units per molecule. In certain embodiments, the polyisobutylene comprises an average of 48 to 54 isobutylene monomeric units per molecule. In certain embodiments, the polyisobutylene comprises an average of 48 to 53 isobutylene monomeric units per molecule. In certain embodiments, the polyisobutylene comprises an average of 48 to 52 isobutylene monomeric units per molecule. In certain embodiments, the polyisobutylene comprises an average of 48 to 51 isobutylene monomeric units per molecule. In certain embodiments, the polyisobutylene comprises an average of 48 to 50 isobutylene monomeric units per molecule. In certain embodiments, the polyisobutylene comprises an average of 50 to 56 isobutylene monomeric units per molecule. In certain embodiments, the polyisobutylene comprises an average of 50 to 55 isobutylene monomeric units per molecule. In certain embodiments, the polyisobutylene comprises an average of 50 to 54 isobutylene monomeric units per molecule. In certain embodiments, the polyisobutylene comprises an average of 50 to 53 isobutylene monomeric units per molecule. In certain embodiments, the polyisobutylene comprises an average of 50 to 52 isobutylene monomeric units per molecule. In certain embodiments, the polyisobutylene comprises an average of 50 to 51 isobutylene monomeric units per molecule. In certain embodiments, the polyisobutylene comprises an average of 51 to 56 isobutylene monomeric units per molecule. In certain embodiments, the polyisobutylene comprises an average of 51 to 55 isobutylene monomeric units per molecule. In certain embodiments, the polyisobutylene comprises an average of 51 to 54 isobutylene monomeric units per molecule. In certain embodiments, the polyisobutylene comprises an average of 51 to 53 isobutylene monomeric units per molecule. In certain embodiments, the polyisobutylene comprises an average of 51 to 52 isobutylene monomeric units per molecule. In certain embodiments, the polyisobutylene comprises an average of 52 to 56 isobutylene monomeric units per molecule. In certain embodiments, the polyisobutylene comprises an average of 52 to 55 isobutylene monomeric units per molecule. In certain embodiments, the polyisobutylene comprises an average of 52 to 54 isobutylene monomeric units per molecule. In certain embodiments, the polyisobutylene comprises an average of 52 to 53 isobutylene monomeric units per molecule. In certain embodiments, the polyisobutylene comprises an average of 53 to 56 isobutylene monomeric units per molecule. In certain embodiments, the polyisobutylene comprises an average of 53 to 55 isobutylene monomeric units per molecule. In certain embodiments, the polyisobutylene comprises an average of 53 to 54 isobutylene monomeric units per molecule. In certain embodiments, the polyisobutylene comprises an average of 54 to 56 isobutylene monomeric units per molecule. In certain embodiments, the polyisobutylene comprises an average of 54 to 55 isobutylene monomeric units per molecule. In certain embodiments, the polyisobutylene comprises an average of 55 to 56 isobutylene monomeric units per molecule. In certain embodiments, the polyisobutylene comprises an average of 3, 4, 5, 6, 7, 8, 9, 10, 12, 14, 16, 18, 20, 22, 24, 26, 28, 30, 32, 34, 36, 38, 40, 42, 44, 46, 48, 50, 51, 52, 53, 54, 55, or 56 isobutylene monomeric units per molecule.

In certain embodiments, the radical initiator may be any suitable radical initiator, and may comprise an organic peroxide initiator or a peracid initiator.

In certain embodiments, the organic peroxide initiator comprises diglutaroyl, dilauroyl peroxide, benzoyl peroxide, dicumyl peroxide, di(tert butyl) peroxide, tertbutyl hydroperoxide, 3,4-dimethyl-3,4-diphenyl-hexane, tert-butylperoxy-2-ethylhexanoate, tert-butylperoxy-benzoate, tert-butylperoxy-pivalate, tert-butylperoxy-3,5,5-trimethyl-hexanoate, di(2,4-dichlorobenzoyl) peroxide, di(2-methylbenzoyl) peroxide, or di(4-tert-butylcyclohexyl) peroxydicarbonate. Half lives of these and other radical initiators are widely available in the relevant scientific literature.

In certain embodiments, the ethylenically unsaturated acylating agent comprises at least one monoethylenically unsaturated C₃-C₂₈ monocarboxylic acid or ester thereof, or at least one C₄-C₂₈ dicarboxylic acid, anhydride or ester thereof.

In certain embodiments, the ethylenically unsaturated acylating agent may comprise one or more compounds falling within the following general formula:

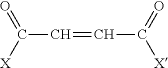

wherein X and X′ are the same or different, provided that at least one of X and X′ is a group that is capable of reacting to esterify alcohols, form amides or amine salts with ammonia or amines, form metal salts with reactive metals or basically reacting metal compounds and otherwise function as acylating agents. In certain embodiments, X and/or X′ is —OH, —O-hydrocarbyl, OM+ where M+ represents one equivalent of a metal, ammonium or amine cation, —NH₂, —Cl, —Br, and/or taken together X and X′ may be —O so as to form an anhydride. In certain embodiments, X and X′ are such that both carboxylic functions can enter into acylation reactions. Suitable reactants include: maleic anhydride; monophenyl maleic anhydride; monomethyl, dimethyl, monochloro, monobromo, monofluoro, dichloro and difluoro maleic anhydride; N-phenylmaleimide and other substituted maleimides, isomaleimides; fumaric acid, maleic acid, alkyl hydrogen maleates and fumarates, dialkyl fumarates and maleates, fumaronilic acids and maleanic acids; and maleonitrile and fumaronitrile.

In certain embodiments, the monoethylenically unsaturated dicarboxylic acids or anhydrides of 3 to 28 carbon atoms may be selected from the group comprising maleic acid, fumaric acid, itaconic acid, mesaconic acid, methylenemalonic acid, citraconic acid, maleic anhydride, itaconic anhydride, citraconic anhydride and methylenemalonic anhydride and mixtures of these with one another.

In certain embodiments, the monoethylenically unsaturated C₃-C₂₈-monocarboxylic acids may be selected from the group comprising acrylic acid, methacrylic acid, dimethacrylic acid, ethylacrylic acid, crotonic acid, allylacetic acid and vinylacetic acid. Another group of suitable monomers is C₁-C₄₀ alkyl esters of monoethylenecially unsaturated C₃-C₁₀ mono- or dicarboxylic acids such as ethyl acrylate, butyl acrylate, 2-ethyl acrylate, decyl acrylate, docedyl acrylate, loctadecyl acrylate and the esters of industrial alcohol mixtures of 14 to 28 carbon atoms, ethyl methacrylate, 2-ethylhexyl methacrylate, decyl methacrylate, octadecyl methacrylate, monobutyl maleate, dibutyl maleate, monodecyl maleate, didodecyl maleate, monooctadecyl maleate, and dioctadecyl maleate.

In certain embodiments, the ethylenically unsaturated acylating agent comprises maleic acid, fumaric acid, itaconic acid, mesaconic acid, methylenemalonic acid, citraconic acid, maleic anhydride, itaconic anhydride, citraconic anhydride, or methylenemalonic anhydride.

In certain embodiments, the radically-functionalized PIBSA product comprises at least one compound of the following general formula I

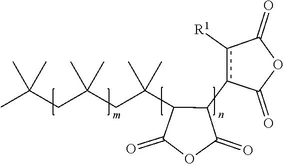

wherein: each R¹ independently represents: —(CH₂)—(C(CH₂))—(CH₃); H; or is not present; wherein when R¹ is not present, the dashed double bond represents a double bond, and when R¹ is present, the dashed double bond represents a single bond; each m independently is an integer from 1 to 52; and each n independently is 0, 1 or 2.

In certain embodiments, each m independently is an integer from 1 to 53, from 1 to 52, from 1 to 51, from 1 to 50, from 1 to 49, from 1 to 48, from 1 to 46, from 1 to 44, from 1 to 42, from 1 to 40, from 1 to 38, from 1 to 36, from 1 to 34, from 1 to 32, from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 7, from 1 to 6, from 1 to 5, from 1 to 4, from 1 to 3, from 1 to 2, from 2 to 54, from 2 to 53, from 2 to 52, from 2 to 51, from 2 to 50, from 2 to 49, from 2 to 48, from 2 to 46, from 2 to 44, from 2 to 42, from 2 to 40, from 2 to 38, from 2 to 36, from 2 to 34, from 2 to 32, from 2 to 30, from 2 to 28, from 2 to 26, from 2 to 24, from 2 to 22, from 2 to 20, from 2 to 18, from 2 to 16, from 2 to 14, from 2 to 12, from 2 to 10, from 2 to 8, from 2 to 7, from 2 to 6, from 2 to 5, from 2 to 4, from 2 to 3, from 3 to 54, from 3 to 53 from 3 to 52, from 3 to 51, from 3 to 50, from 3 to 49, from 3 to 48, from 3 to 46, from 3 to 44, from 3 to 42, from 3 to 40, from 3 to 38, from 3 to 36, from 3 to 34, from 3 to 32, from 3 to 30, from 3 to 28, from 3 to 26, from 3 to 24, from 3 to 22, from 3 to 20, from 3 to 18, from 3 to 16, from 3 to 14, from 3 to 12, from 3 to 10, from 3 to 8, from 3 to 7, from 3 to 6, from 3 to 5, from 3 to 4, from 4 to 54, from 4 to 53, from 4 to 52, from 4 to 51, from 4 to 50, from 4 to 49, from 4 to 48, from 4 to 46, from 4 to 44, from 4 to 42, from 4 to 40, from 4 to 38, from 4 to 36, from 4 to 34, from 4 to 32, from 4 to 30, from 4 to 28, from 4 to 26, from 4 to 24, from 4 to 22, from 4 to 20, from 4 to 18, from 4 to 16, from 4 to 14, from 4 to 12, from 4 to 10, from 4 to 8, from 4 to 7, from 4 to 6, from 4 to 5, from 5 to 54, from 5 to 53, from 5 to 52, from 5 to 51, from 5 to 50, from 5 to 49, from 5 to 48, from 5 to 46, from 5 to 44, from 5 to 42, from 5 to 40, from 5 to 38, from 5 to 36, from 5 to 34, from 5 to 32, from 5 to 30, from 5 to 28, from 5 to 26, from 5 to 24, from 5 to 22, from 5 to 20, from 5 to 18, from 5 to 16, from 5 to 14, from 5 to 12, from 5 to 10, from 5 to 8, from 5 to 7, from 5 to 6, from 6 to 54, from 6 to 53, from 6 to 52, from 6 to 51, from 6 to 50, from 6 to 49, from 6 to 48, from 6 to 46, from 6 to 44, from 6 to 42, from 6 to 40, from 6 to 38, from 6 to 36, from 6 to 34, from 6 to 32, from 6 to 30, from 6 to 28, from 6 to 26, from 6 to 24, from 6 to 22, from 6 to 20, from 6 to 18, from 6 to 16, from 6 to 14, from 6 to 12, from 6 to 10, from 6 to 8, from 6 to 7, from 7 to 54, from 7 to 53, from 7 to 52, from 7 to 51, from 7 to 50, from 7 to 49, from 7 to 48, from 7 to 46, from 7 to 44, from 7 to 42, from 7 to 40, from 7 to 38, from 7 to 36, from 7 to 34, from 7 to 32, from 7 to 30, from 7 to 28, from 7 to 26, from 7 to 24, from 7 to 22, from 7 to 20, from 7 to 18, from 7 to 16, from 7 to 14, from 7 to 12, from 7 to 10, from 7 to 8, from 8 to 54, from 8 to 53, from 8 to 52, from 8 to 51, from 8 to 50, from 8 to 49, from 8 to 48, from 8 to 46, from 8 to 44, from 8 to 42, from 8 to 40, from 8 to 38, from 8 to 36, from 8 to 34, from 8 to 32, from 8 to 30, from 8 to 28, from 8 to 26, from 8 to 24, from 8 to 22, from 8 to 20, from 8 to 18, from 8 to 16, from 8 to 14, from 8 to 12, from 8 to 10, from 10 to 54, from 10 to 53, from 10 to 52, from 10 to 51, from 10 to 50, from 10 to 49, from 10 to 48, from 10 to 46, from 10 to 44, from 10 to 42, from 10 to 40, from 10 to 38, from 10 to 36, from 10 to 34, from 10 to 32, from 10 to 30, from 10 to 28, from 10 to 26, from 10 to 24, from 10 to 22, from 10 to 20, from 10 to 18, from 10 to 16, from 10 to 14, from 10 to 12, from 12 to 54, from 12 to 53, from 12 to 52, from 12 to 51, from 12 to 50, from 12 to 49, from 12 to 48, from 12 to 46, from 12 to 44, from 12 to 42, from 12 to 40, from 12 to 38, from 12 to 36, from 12 to 34, from 12 to 32, from 12 to 30, from 12 to 28, from 12 to 26, from 12 to 24, from 12 to 22, from 12 to 20, from 12 to 18, from 12 to 16, from 12 to 14, from 14 to 54, from 14 to 53, from 14 to 52, from 14 to 51, from 14 to 50, from 14 to 49, from 14 to 48, from 14 to 46, from 14 to 44, from 14 to 42, from 14 to 40, from 14 to 38, from 14 to 36, from 14 to 34, from 14 to 32, from 14 to 30, from 14 to 28, from 14 to 26, from 14 to 24, from 14 to 22, from 14 to 20, from 14 to 18, from 14 to 16, from 16 to 54, from 16 to 53, from 16 to 52, from 16 to 51, from 16 to 50, from 16 to 49, from 16 to 48, from 16 to 46, from 16 to 44, from 16 to 42, from 16 to 40, from 16 to 38, from 16 to 36, from 16 to 34, from 16 to 32, from 16 to 30, from 16 to 28, from 16 to 26, from 16 to 24, from 16 to 22, from 16 to 20, from 16 to 18, from 18 to 54, from 18 to 53, from 18 to 52, from 18 to 51, from 18 to 50, from 18 to 49, from 18 to 48, from 18 to 46, from 18 to 44, from 18 to 42, from 18 to 40, from 18 to 38, from 18 to 36, from 18 to 34, from 18 to 32, from 18 to 30, from 18 to 28, from 18 to 26, from 18 to 24, from 18 to 22, from 18 to 20, from 20 to 54, from 20 to 53, from 20 to 52, from 20 to 51, from 20 to 50, from 20 to 49, from 20 to 48, from 20 to 46, from 20 to 44, from 20 to 42, from 20 to 40, from 20 to 38, from 20 to 36, from 20 to 34, from 20 to 32, from 20 to 30, from 20 to 28, from 20 to 26, from 20 to 24, from 20 to 22, from 22 to 54, from 22 to 53, from 22 to 52, from 22 to 51, from 22 to 50, from 22 to 49, from 22 to 48, from 22 to 46, from 22 to 44, from 22 to 42, from 22 to 40, from 22 to 38, from 22 to 36, from 22 to 34, from 22 to 32, from 22 to 30, from 22 to 28, from 22 to 26, from 22 to 24, from 24 to 54, from 24 to 53, from 24 to 52, from 24 to 51, from 24 to 50, from 24 to 49, from 24 to 48, from 24 to 46, from 24 to 44, from 24 to 42, from 24 to 40, from 24 to 38, from 24 to 36, from 24 to 34, from 24 to 32, from 24 to 30, from 24 to 28, from 24 to 26, from 26 to 54, from 26 to 53, from 26 to 52, from 26 to 51, from 26 to 50, from 26 to 49, from 26 to 48, from 26 to 46, from 26 to 44, from 26 to 42, from 26 to 40, from 26 to 38, from 26 to 36, from 26 to 34, from 26 to 32, from 26 to 30, from 26 to 28, from 28 to 54, from 28 to 53, from 28 to 52, from 28 to 51, from 28 to 50, from 28 to 49, from 28 to 48, from 28 to 46, from 28 to 44, from 28 to 42, from 28 to 40, from 28 to 38, from 28 to 36, from 28 to 34, from 28 to 32, from 28 to 30, from 30 to 54, from 30 to 53, from 30 to 52, from 30 to 51, from 30 to 50, from 30 to 49, from 30 to 48, from 30 to 46, from 30 to 44, from 30 to 42, from 30 to 40, from 30 to 38, from 30 to 36, from 30 to 34, from 30 to 32, from 32 to 54, from 32 to 53, from 32 to 52, from 32 to 51, from 32 to 50, from 32 to 49, from 32 to 48, from 32 to 46, from 32 to 44, from 32 to 42, from 32 to 40, from 32 to 38, from 32 to 36, from 32 to 34, from 34 to 54, from 34 to 53, from 34 to 52, from 34 to 51, from 34 to 50, from 34 to 49, from 34 to 48, from 34 to 46, from 34 to 44, from 34 to 42, from 34 to 40, from 34 to 38, from 34 to 36, from 36 to 54, from 36 to 53, from 36 to 52, from 36 to 51, from 36 to 50, from 36 to 49, from 36 to 48, from 36 to 46, from 36 to 44, from 36 to 42, from 36 to 40, from 36 to 38, from 38 to 54, from 38 to 53, from 38 to 52, from 38 to 51, from 38 to 50, from 38 to 49, from 38 to 48, from 38 to 46, from 38 to 44, from 38 to 42, from 38 to 40, from 40 to 54, from 40 to 53, from 40 to 52, from 40 to 51, from 40 to 50, from 40 to 49, from 40 to 48, from 40 to 46, from 40 to 44, from 40 to 42, from 42 to 54, from 42 to 53, from 42 to 52, from 42 to 51, from 42 to 50, from 42 to 49, from 42 to 48, from 42 to 46, from 42 to 44, from 44 to 54, from 44 to 53, from 44 to 52, from 44 to 51, from 44 to 50, from 44 to 49, from 44 to 48, from 44 to 46, from 46 to 54, from 46 to 53, from 46 to 52, from 46 to 51, from 46 to 50, from 46 to 49, from 46 to 48, from 48 to 54, from 48 to 53, from 48 to 52, from 48 to 51, from 48 to 50, from 48 to 49, from 49 to 54, from 49 to 53, from 49 to 52, from 49 to 51, from 49 to 50, from 50 to 54, from 50 to 53, from 50 to 52, from 50 to 51, from 51 to 54, from 51 to 53, from 51 to 52, from 51 to 51, from 52 to 54, from 52 to 53, or from 53 to 54. In certain embodiments, each m independently is 1, 2, 3, 4, 5, 6, 7, 8, 10, 12, 14, 16, 18, 20, 22, 24, 26, 28, 30, 32, 34, 36, 38, 40, 42, 44, 46, 48, 49, 50, 51, 52, 53, or 54.

In certain embodiments, the radically-functionalized PIBSA product comprises at least 30 (such as at least 35, 40, 45, 50, 55, 60, 65, 70, 75, 80, 85, 90, 95, or 99) mol % of the at least one compound of the general formula I. In certain embodiments, the radically-functionalized PIBSA product comprises from 30 to 100 (such as from 35 to 100, from 40 to 100, from 45 to 100, from 50 to 100, from 55 to 100, from 60 to 100, from 65 to 100, from 70 to 100, from 75 to 100, from 80 to 100, from 85 to 100, from 90 to 100, from 95 to 100, from 99 to 100, from 30 to 99, from 35 to 99, from 40 to 99, from 45 to 99, from 50 to 99, from 55 to 99, from 60 to 99, from 65 to 99, from 70 to 99, from 75 to 99, from 80 to 99, from 85 to 99, from 90 to 99, from 95 to 99, from 30 to 95, from 35 to 95, from 40 to 95, from 45 to 95, from 50 to 95, from 55 to 95, from 60 to 95, from 65 to 95, from 70 to 95, from 75 to 95, from 80 to 95, from 85 to 95, from 90 to 95, from 30 to 90, from 35 to 90, from 40 to 90, from 45 to 90, from 50 to 90, from 55 to 90, from 60 to 90, from 65 to 90, from 70 to 90, from 75 to 90, from 80 to 90, from 85 to 90, from 30 to 85, from 35 to 85, from 40 to 85, from 45 to 85, from 50 to 85, from 55 to 85, from 60 to 85, from 65 to 85, from 70 to 85, from 75 to 85, from 80 to 85, from 30 to 80, from 35 to 80, from 40 to 80, from 45 to 80, from 50 to 80, from 55 to 80, from 60 to 80, from 65 to 80, from 70 to 80, from 75 to 80, from 30 to 75, from 35 to 75, from 40 to 75, from 45 to 75, from 50 to 75, from 55 to 75, from 60 to 75, from 65 to 75, from 70 to 75, from 30 to 70, from 35 to 70, from 40 to 70, from 45 to 70, from 50 to 70, from 55 to 70, from 60 to 70, from 65 to 70, from 30 to 65, from 35 to 65, from 40 to 65, from 45 to 65, from 50 to 65, from 55 to 65, from 60 to 65, from 30 to 60, from 35 to 60, from 40 to 60, from 45 to 60, from 50 to 60, from 55 to 60, from 30 to 55, from 35 to 55, from 40 to 55, from 45 to 55, from 50 to 55, from 30 to 50, from 35 to 50, from 40 to 50, from 45 to 50, from 30 to 45, from 35 to 45, from 40 to 45, from 30 to 40, from 35 to 40 or from 30 to 35) mol % of the at least one compound of the general formula I.

In certain embodiments, the radically-functionalized PIBSA product comprises less than 40 (such as less than 35, 30, 25, 20, 15, 14, 13, 12, 11, 10, 9, 8, 7, 6, 5, 4, 3, 2, or 1) weight percent unreacted polyisobutylene. In certain embodiments, the radically-functionalized PIBSA product comprises from 0 to 40 (such as from 0 to 35, from 0 to 30, from 0 to 25, from 0 to 20, from 0 to 15, from 0 to 14, from 0 to 13, from 0 to 12, from 0 to 11, from 0 to 10, from 0 to 9, from 0 to 8, from 0 to 7, from 0 to 6, from 0 to 5, from 0 to 4, from 0 to 3, from 0 to 2, from 0 to 1, from greater than 0 to 40, from greater than 0 to 35, from greater than 0 to 30, from greater than 0 to 25, from greater than 0 to 20, from greater than 0 to 15, from greater than 0 to 14, from greater than 0 to 13, from greater than 0 to 12, from greater than 0 to 11, from greater than 0 to 10, from greater than 0 to 9, from greater than 0 to 8, from greater than 0 to 7, from greater than 0 to 6, from greater than 0 to 5, from greater than 0 to 4, from greater than 0 to 3, from greater than 0 to 2, from greater than 0 to 1, from greater than 0 to 35, from 1 to 40, from 1 to 35, from 1 to 30, from 1 to 25, from 1 to 20, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, from 2 to 40, from 2 to 35, from 2 to 30, from 2 to 25, from 2 to 20, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, from 2 to 3, from 3 to 40, from 3 to 35, from 3 to 30, from 3 to 25, from 3 to 20, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4, from 4 to 40, from 4 to 35, from 4 to 30, from 4 to 25, from 4 to 20, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 5 to 40, from 5 to 35, from 5 to 30, from 5 to 25, from 5 to 20, from 5 to 15, from 5 to 14, from 5 to 13, from 5 to 12, from 5 to 11, from 5 to 10, from 5 to 9, from 5 to 8, from 5 to 7, from 5 to 6, from 6 to 40, from 6 to 35, from 6 to 30, from 6 to 25, from 6 to 20, from 6 to 15, from 6 to 14, from 6 to 13, from 6 to 12, from 6 to 11, from 6 to 10, from 6 to 9, from 6 to 8, from 6 to 7, from 7 to 40, from 7 to 35, from 7 to 30, from 7 to 25, from 7 to 20, from 7 to 15, from 7 to 14, from 7 to 13, from 7 to 12, from 7 to 11, from 7 to 10, from 7 to 9, from 7 to 8, from 8 to 40, from 8 to 35, from 8 to 30, from 8 to 25, from 8 to 20, from 8 to 15, from 8 to 14, from 8 to 13, from 8 to 12, from 8 to 11, from 8 to 10, from 8 to 9, from 9 to 40, from 9 to 35, from 9 to 30, from 9 to 25, from 9 to 20, from 9 to 15, from 9 to 14, from 9 to 13, from 9 to 12, from 9 to 11, from 9 to 10, from 10 to 40, from 10 to 35, from 10 to 30, from 10 to 25, from 10 to 20, from 10 to 15, from 10 to 14, from 10 to 13, from 10 to 12, from 10 to 11, from 11 to 40, from 11 to 35, from 11 to 30, from 11 to 25, from 11 to 20, from 11 to 15, from 11 to 14, from 11 to 13, from 11 to 12, from 12 to 40, from 12 to 35, from 12 to 30, from 12 to 25, from 12 to 20, from 12 to 15, from 12 to 14, from 12 to 13, from 13 to 40, from 13 to 35, from 13 to 30, from 13 to 25, from 13 to 20, from 13 to 15, from 13 to 14, from 14 to 40, from 14 to 35, from 14 to 30, from 14 to 25, from 14 to 20, from 14 to 15, from 15 to 40, from 15 to 35, from 15 to 30, from 15 to 25, from 15 to 20, from 20 to 40, from 20 to 35, from 20 to 30, from 20 to 25, from 25 to 40, from 25 to 35, from 25 to 30, from 30 to 40, from 30 to 35, or from 35 to 40) weight percent unreacted polyisobutylene.

Also provided are embodiments of the processes to produce the radically-functionalized PIBSA product described above, wherein the process comprises reacting polyisobutylene with a radical initiator and an ethylenically unsaturated acylating agent in two or more additions at a reaction temperature of from 150 °C to 225 °C to produce the radically-functionalized PIBSA product. In certain embodiments, the radical initiator and the ethylenically unsaturated acylating agent are added in two to ten additions (such as two to nine, two to eight, two to seven, two to six, two to five, two to four, two to three, or three additions).

In certain embodiments, each of the two or more additions independently comprises at least 5 (such as at least 10, 15, 20, 25, 30, 35, 40, 45, or 50) weight percent of the total amount of the radical initiator and/or at least 5 (such as at least 10, 15, 20, 25, 30, 35, 40, 45, or 50) weight percent of the total amount of the ethylenically unsaturated acylating agent to be reacted with the polyisobutylene. In certain embodiments, each of the two or more additions independently comprises at most 95 (such as at most 90, 85, 80, 75, 70, 65, 60, 55, 50, 45, 40, 35, 30, 25, 20, 15 or 10) weight percent of the total amount of the radical initiator and/or at most 95 (such as at most 90, 85, 80, 75, 70, 65, 60, 55, 50, 45, 40, 35, 30, 25, 20, 15 or 10) weight percent of the total amount of the ethylenically unsaturated acylating agent to be reacted with the polyisobutylene. In certain embodiments, each of the two or more additions independently comprises from 5 to 95 (such as from 5 to 90, from 5 to 85, from 5 to 80, from 5 to 75, from 5 to 70, from 5 to 65, from 5 to 60, from 5 to 55, from 5 to 50, from 5 to 45, from 5 to 40, from 5 to 35, from 5 to 30, from 5 to 25, from 5 to 20, from 5 to 15, from 5 to 10, from 10 to 95, from 10 to 90, from 10 to 85, from 10 to 80, from 10 to 75, from 10 to 70, from 10 to 65, from 10 to 60, from 10 to 55, from 10 to 50, from 10 to 45, from 10 to 40, from 10 to 35, from 10 to 30, from 10 to 25, from 10 to 20, from 10 to 15, from 15 to 95, from 15 to 90, from 15 to 85, from 15 to 80, from 15 to 75, from 15 to 70, from 15 to 65, from 15 to 60, from 15 to 55, from 15 to 50, from 15 to 45, from 15 to 40, from 15 to 35, from 15 to 30, from 15 to 25, from 15 to 20, from 20 to 95, from 20 to 90, from 20 to 85, from 20 to 80, from 20 to 75, from 20 to 70, from 20 to 65, from 20 to 60, from 20 to 55, from 20 to 50, from 20 to 45, from 20 to 40, from 20 to 35, from 20 to 30, from 20 to 25, from 25 to 95, from 25 to 90, from 25 to 85, from 25 to 80, from 25 to 75, from 25 to 70, from 25 to 65, from 25 to 60, from 25 to 55, from 25 to 50, from 25 to 45, from 25 to 40, from 25 to 35, from 25 to 30, from 30 to 95, from 30 to 90, from 30 to 85, from 30 to 80, from 30 to 75, from 30 to 70, from 30 to 65, from 30 to 60, from 30 to 55, from 30 to 50, from 30 to 45, from 30 to 40, from 30 to 35, from 35 to 95, from 35 to 90, from 35 to 85, from 35 to 80, from 35 to 75, from 35 to 70, from 35 to 65, from 35 to 60, from 35 to 55, from 35 to 50, from 35 to 45, from 35 to 40, from 40 to 95, from 40 to 90, from 40 to 85, from 40 to 80, from 40 to 75, from 40 to 70, from 40 to 65, from 40 to 60, from 40 to 55, from 40 to 50, from 40 to 45, from 45 to 95, from 45 to 90, from 45 to 85, from 45 to 80, from 45 to 75, from 45 to 70, from 45 to 65, from 45 to 60, from 45 to 45, from 45 to 50, from 50 to 95, from 50 to 90, from 50 to 85, from 50 to 80, from 50 to 75, from 50 to 70, from 50 to 65, from 50 to 60, or from 50 to 55) weight percent of the total amount of the radical initiator and/or from 5 to 95 (such as from 5 to 90, from 5 to 85, from 5 to 80, from 5 to 75, from 5 to 70, from 5 to 65, from 5 to 60, from 5 to 55, from 5 to 50, from 5 to 45, from 5 to 40, from 5 to 35, from 5 to 30, from 5 to 25, from 5 to 20, from 5 to 15, from 5 to 10, from 10 to 95, from 10 to 90, from 10 to 85, from 10 to 80, from 10 to 75, from 10 to 70, from 10 to 65, from 10 to 60, from 10 to 55, from 10 to 50, from 10 to 45, from 10 to 40, from 10 to 35, from 10 to 30, from 10 to 25, from 10 to 20, from 10 to 15, from 15 to 95, from 15 to 90, from 15 to 85, from 15 to 80, from 15 to 75, from 15 to 70, from 15 to 65, from 15 to 60, from 15 to 55, from 15 to 50, from 15 to 45, from 15 to 40, from 15 to 35, from 15 to 30, from 15 to 25, from 15 to 20, from 20 to 95, from 20 to 90, from 20 to 85, from 20 to 80, from 20 to 75, from 20 to 70, from 20 to 65, from 20 to 60, from 20 to 55, from 20 to 50, from 20 to 45, from 20 to 40, from 20 to 35, from 20 to 30, from 20 to 25, from 25 to 95, from 25 to 90, from 25 to 85, from 25 to 80, from 25 to 75, from 25 to 70, from 25 to 65, from 25 to 60, from 25 to 55, from 25 to 50, from 25 to 45, from 25 to 40, from 25 to 35, from 25 to 30, from 30 to 95, from 30 to 90, from 30 to 85, from 30 to 80, from 30 to 75, from 30 to 70, from 30 to 65, from 30 to 60, from 30 to 55, from 30 to 50, from 30 to 45, from 30 to 40, from 30 to 35, from 35 to 95, from 35 to 90, from 35 to 85, from 35 to 80, from 35 to 75, from 35 to 70, from 35 to 65, from 35 to 60, from 35 to 55, from 35 to 50, from 35 to 45, from 35 to 40, from 40 to 95, from 40 to 90, from 40 to 85, from 40 to 80, from 40 to 75, from 40 to 70, from 40 to 65, from 40 to 60, from 40 to 55, from 40 to 50, from 40 to 45, from 45 to 95, from 45 to 90, from 45 to 85, from 45 to 80, from 45 to 75, from 45 to 70, from 45 to 65, from 45 to 60, from 45 to 45, from 45 to 50, from 50 to 95, from 50 to 90, from 50 to 85, from 50 to 80, from 50 to 75, from 50 to 70, from 50 to 65, from 50 to 60, or from 50 to 55) weight percent of the total amount of the ethylenically unsaturated acylating agent to be reacted with the polyisobutylene.

In certain embodiments, at least two of the two or more additions are separated in time by at least the half-life of the radical initiator. In certain embodiments, at least two of the two or more additions are separated in time by at least 1.1 (such as at least 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.2, 4.4, 4.6, 4.8, 5, 5.5, 6, 6.5, 7, 7.5, 8, 8.5, 9, 9.5 or 10) times the half-life of the radical initiator. In certain embodiments, each of the two or more additions are separated in times by from 1 to 10 (such as from 1.1 to 10, from 1.2 to 10, from 1.3 to 10, from 1.4 to 10, from 1.5 to 10, from 1.6 to 10, from 1.7 to 10, from 1.8 to 10, from 1.9 to 10, from 2 to 10, from 2.1 to 10, from 2.2 to 10, from 2.3 to 10, from 2.4 to 10, from 2.5 to 10, from 2.6 to 10, from 2.7 to 10, from 2.8 to 10, from 2.9 to 10, from 3 to 10, from 3.1 to 10, from 3.2 to 10, from 3.3 to 10, from 3.4 to 10, from 3.5 to 10, from 3.6 to 10, from 3.7 to 10, from 3.8 to 10, from 3.9 to 10, from 4 to 10, from 4.2 to 10, from 4.4 to 10, from 4.6 to 10, from 4.8 to 10, from 5 to 10, from 5.5 to 10, from 6 to 10, from 6.5 to 10, from 7 to 10, from 7.5 to 10, from 8 to 10, from 8.5 to 10, from 9 to 10, from 9.5 to 10, from 1 to 9.5, from 1.1 to 9.5, from 1.2 to 9.5, from 1.3 to 9.5, from 1.4 to 9.5, from 1.5 to 9.5, from 1.6 to 9.5, from 1.7 to 9.5, from 1.8 to 9.5, from 1.9 to 9.5, from 2 to 9.5, from 2.1 to 9.5, from 2.2 to 9.5, from 2.3 to 9.5, from 2.4 to 9.5, from 2.5 to 9.5, from 2.6 to 9.5, from 2.7 to 9.5, from 2.8 to 9.5, from 2.9 to 9.5, from 3 to 9.5, from 3.1 to 9.5, from 3.2 to 9.5, from 3.3 to 9.5, from 3.4 to 9.5, from 3.5 to 9.5, from 3.6 to 9.5, from 3.7 to 9.5, from 3.8 to 9.5, from 3.9 to 9.5, from 4 to 9.5, from 4.2 to 9.5, from 4.4 to 9.5, from 4.6 to 9.5, from 4.8 to 9.5, from 5 to 9.5, from 5.5 to 9.5, from 6 to 9.5, from 6.5 to 9.5, from 7 to 9.5, from 7.5 to 9.5, from 8 to 9.5, from 8.5 to 9.5, from 9 to 9.5, from 1 to 9, from 1.1 to 9, from 1.2 to 9, from 1.3 to 9, from 1.4 to 9, from 1.5 to 9, from 1.6 to 9, from 1.7 to 9, from 1.8 to 9, from 1.9 to 9, from 2 to 9, from 2.1 to 9, from 2.2 to 9, from 2.3 to 9, from 2.4 to 9, from 2.5 to 9, from 2.6 to 9, from 2.7 to 9, from 2.8 to 9, from 2.9 to 9, from 3 to 9, from 3.1 to 9, from 3.2 to 9, from 3.3 to 9, from 3.4 to 9, from 3.5 to 9, from 3.6 to 9, from 3.7 to 9, from 3.8 to 9, from 3.9 to 9, from 4 to 9, from 4.2 to 9, from 4.4 to 9, from 4.6 to 9, from 4.8 to 9, from 5 to 9, from 5.5 to 9, from 6 to 9, from 6.5 to 9, from 7 to 9, from 7.5 to 9, from 8 to 9, from 8.5 to 9, from 1 to 8.5, from 1.1 to 8.5, from 1.2 to 8.5, from 1.3 to 8.5, from 1.4 to 8.5, from 1.5 to 8.5, from 1.6 to 8.5, from 1.7 to 8.5, from 1.8 to 8.5, from 1.9 to 8.5, from 2 to 8.5, from 2.1 to 8.5, from 2.2 to 8.5, from 2.3 to 8.5, from 2.4 to 8.5, from 2.5 to 8.5, from 2.6 to 8.5, from 2.7 to 8.5, from 2.8 to 8.5, from 2.9 to 8.5, from 3 to 8.5, from 3.1 to 8.5, from 3.2 to 8.5, from 3.3 to 8.5, from 3.4 to 8.5, from 3.5 to 8.5, from 3.6 to 8.5, from 3.7 to 8.5, from 3.8 to 8.5, from 3.9 to 8.5, from 4 to 8.5, from 4.2 to 8.5, from 4.4 to 8.5, from 4.6 to 8.5, from 4.8 to 8.5, from 5 to 8.5, from 5.5 to 8.5, from 6 to 8.5, from 6.5 to 8.5, from 7 to 8.5, from 7.5 to 8.5, from 8 to 8.5, from 1 to 8, from 1.1 to 8, from 1.2 to 8, from 1.3 to 8, from 1.4 to 8, from 1.5 to 8, from 1.6 to 8, from 1.7 to 8, from 1.8 to 8, from 1.9 to 8, from 2 to 8, from 2.1 to 8, from 2.2 to 8, from 2.3 to 8, from 2.4 to 8, from 2.5 to 8, from 2.6 to 8, from 2.7 to 8, from 2.8 to 8, from 2.9 to 8, from 3 to 8, from 3.1 to 8, from 3.2 to 8, from 3.3 to 8, from 3.4 to 8, from 3.5 to 8, from 3.6 to 8, from 3.7 to 8, from 3.8 to 8, from 3.9 to 8, from 4 to 8, from 4.2 to 8, from 4.4 to 8, from 4.6 to 8, from 4.8 to 8, from 5 to 8, from 5.5 to 8, from 6 to 8, from 6.5 to 8, from 7 to 8, from 7.5 to 8, from 1 to 7.5, from 1.1 to 7.5, from 1.2 to 7.5, from 1.3 to 7.5, from 1.4 to 7.5, from 1.5 to 7.5, from 1.6 to 7.5, from 1.7 to 7.5, from 1.8 to 7.5, from 1.9 to 7.5, from 2 to 7.5, from 2.1 to 7.5, from 2.2 to 7.5, from 2.3 to 7.5, from 2.4 to 7.5, from 2.5 to 7.5, from 2.6 to 7.5, from 2.7 to 7.5, from 2.8 to 7.5, from 2.9 to 7.5, from 3 to 7.5, from 3.1 to 7.5, from 3.2 to 7.5, from 3.3 to 7.5, from 3.4 to 7.5, from 3.5 to 7.5, from 3.6 to 7.5, from 3.7 to 7.5, from 3.8 to 7.5, from 3.9 to 7.5, from 4 to 7.5, from 4.2 to 7.5, from 4.4 to 7.5, from 4.6 to 7.5, from 4.8 to 7.5, from 5 to 7.5, from 5.5 to 7.5, from 6 to 7.5, from 6.5 to 7.5, from 7 to 7.5, from 1 to 7, from 1.1 to 7, from 1.2 to 7, from 1.3 to 7, from 1.4 to 7, from 1.5 to 7, from 1.6 to 7, from 1.7 to 7, from 1.8 to 7, from 1.9 to 7, from 2 to 7, from 2.1 to 7, from 2.2 to 7, from 2.3 to 7, from 2.4 to 7, from 2.5 to 7, from 2.6 to 7, from 2.7 to 7, from 2.8 to 7, from 2.9 to 7, from 3 to 7, from 3.1 to 7, from 3.2 to 7, from 3.3 to 7, from 3.4 to 7, from 3.5 to 7, from 3.6 to 7, from 3.7 to 7, from 3.8 to 7, from 3.9 to 7, from 4 to 7, from 4.2 to 7, from 4.4 to 7, from 4.6 to 7, from 4.8 to 7, from 5 to 7, from 5.5 to 7, from 6 to 7, from 6.5 to 7, from 1 to 6.5, from 1.1 to 6.5, from 1.2 to 6.5, from 1.3 to 6.5, from 1.4 to 6.5, from 1.5 to 6.5, from 1.6 to 6.5, from 1.7 to 6.5, from 1.8 to 6.5, from 1.9 to 6.5, from 2 to 6.5, from 2.1 to 6.5, from 2.2 to 6.5, from 2.3 to 6.5, from 2.4 to 6.5, from 2.5 to 6.5, from 2.6 to 6.5, from 2.7 to 6.5, from 2.8 to 6.5, from 2.9 to 6.5, from 3 to 6.5, from 3.1 to 6.5, from 3.2 to 6.5, from 3.3 to 6.5, from 3.4 to 6.5, from 3.5 to 6.5, from 3.6 to 6.5, from 3.7 to 6.5, from 3.8 to 6.5, from 3.9 to 6.5, from 4 to 6.5, from 4.2 to 6.5, from 4.4 to 6.5, from 4.6 to 6.5, from 4.8 to 6.5, from 5 to 6.5, from 5.5 to 6.5, from 6 to 6.5, from 1 to 6, from 1.1 to 6, from 1.2 to 6, from 1.3 to 6, from 1.4 to 6, from 1.5 to 6, from 1.6 to 6, from 1.7 to 6, from 1.8 to 6, from 1.9 to 6, from 2 to 6, from 2.1 to 6, from 2.2 to 6, from 2.3 to 6, from 2.4 to 6, from 2.5 to 6, from 2.6 to 6, from 2.7 to 6, from 2.8 to 6, from 2.9 to 6, from 3 to 6, from 3.1 to 6, from 3.2 to 6, from 3.3 to 6, from 3.4 to 6, from 3.5 to 6, from 3.6 to 6, from 3.7 to 6, from 3.8 to 6, from 3.9 to 6, from 4 to 6, from 4.2 to 6, from 4.4 to 6, from 4.6 to 6, from 4.8 to 6, from 5 to 6, from 5.5 to 6, from 1 to 5.5, from 1.1 to 5.5, from 1.2 to 5.5, from 1.3 to 5.5, from 1.4 to 5.5, from 1.5 to 5.5, from 1.6 to 5.5, from 1.7 to 5.5, from 1.8 to 5.5, from 1.9 to 5.5, from 2 to 5.5, from 2.1 to 5.5, from 2.2 to 5.5, from 2.3 to 5.5, from 2.4 to 5.5, from 2.5 to 5.5, from 2.6 to 5.5, from 2.7 to 5.5, from 2.8 to 5.5, from 2.9 to 5.5, from 3 to 5.5, from 3.1 to 5.5, from 3.2 to 5.5, from 3.3 to 5.5, from 3.4 to 5.5, from 3.5 to 5.5, from 3.6 to 5.5, from 3.7 to 5.5, from 3.8 to 5.5, from 3.9 to 5.5, from 4 to 5.5, from 4.2 to 5.5, from 4.4 to 5.5, from 4.6 to 5.5, from 4.8 to 5.5, from 5 to 5.5, from 1 to 5, from 1.1 to 5, from 1.2 to 5, from 1.3 to 5, from 1.4 to 5, from 1.5 to 5, from 1.6 to 5, from 1.7 to 5, from 1.8 to 5, from 1.9 to 5, from 2 to 5, from 2.1 to 5, from 2.2 to 5, from 2.3 to 5, from 2.4 to 5, from 2.5 to 5, from 2.6 to 5, from 2.7 to 5, from 2.8 to 5, from 2.9 to 5, from 3 to 5, from 3.1 to 5, from 3.2 to 5, from 3.3 to 5, from 3.4 to 5, from 3.5 to 5, from 3.6 to 5, from 3.7 to 5, from 3.8 to 5, from 3.9 to 5, from 4 to 5, from 4.2 to 5, from 4.4 to 5, from 4.6 to 5, from 4.8 to 5, from 1 to 4.8, from 1.1 to 4.8, from 1.2 to 4.8, from 1.3 to 4.8, from 1.4 to 4.8, from 1.5 to 4.8, from 1.6 to 4.8, from 1.7 to 4.8, from 1.8 to 4.8, from 1.9 to 4.8, from 2 to 4.8, from 2.1 to 4.8, from 2.2 to 4.8, from 2.3 to 4.8, from 2.4 to 4.8, from 2.5 to 4.8, from 2.6 to 4.8, from 2.7 to 4.8, from 2.8 to 4.8, from 2.9 to 4.8, from 3 to 4.8, from 3.1 to 4.8, from 3.2 to 4.8, from 3.3 to 4.8, from 3.4 to 4.8, from 3.5 to 4.8, from 3.6 to 4.8, from 3.7 to 4.8, from 3.8 to 4.8, from 3.9 to 4.8, from 4 to 4.8, from 4.2 to 4.8, from 4.4 to 4.8, from 4.6 to 4.8, from 1. to 4.6, from 1.1 to 4.6, from 1.2 to 4.6, from 1.3 to 4.6, from 1.4 to 4.6, from 1.5 to 4.6, from 1.6 to 4.6, from 1.7 to 4.6, from 1.8 to 4.6, from 1.9 to 4.6, from 2 to 4.6, from 2.1 to 4.6, from 2.2 to 4.6, from 2.3 to 4.6, from 2.4 to 4.6, from 2.5 to 4.6, from 2.6 to 4.6, from 2.7 to 4.6, from 2.8 to 4.6, from 2.9 to 4.6, from 3 to 4.6, from 3.1 to 4.6, from 3.2 to 4.6, from 3.3 to 4.6, from 3.4 to 4.6, from 3.5 to 4.6, from 3.6 to 4.6, from 3.7 to 4.6, from 3.8 to 4.6, from 3.9 to 4.6, from 4 to 4.6, from 4.2 to 4.6, from 4.4 to 4.6, from 1 to 4.4, from 1.1 to 4.4, from 1.2 to 4.4, from 1.3 to 4.4, from 1.4 to 4.4, from 1.5 to 4.4, from 1.6 to 4.4, from 1.7 to 4.4, from 1.8 to 4.4, from 1.9 to 4.4, from 2 to 4.4, from 2.1 to 4.4, from 2.2 to 4.4, from 2.3 to 4.4, from 2.4 to 4.4, from 2.5 to 4.4, from 2.6 to 4.4, from 2.7 to 4.4, from 2.8 to 4.4, from 2.9 to 4.4, from 3 to 4.4, from 3.1 to 4.4, from 3.2 to 4.4, from 3.3 to 4.4, from 3.4 to 4.4, from 3.5 to 4.4, from 3.6 to 4.4, from 3.7 to 4.4, from 3.8 to 4.4, from 3.9 to 4.4, from 4 to 4.4, from 4.2 to 4.4, from 1 to 4.2, from 1.1 to 4.2, from 1.2 to 4.2, from 1.3 to 4.2, from 1.4 to 4.2, from 1.5 to 4.2, from 1.6 to 4.2, from 1.7 to 4.2, from 1.8 to 4.2, from 1.9 to 4.2, from 2 to 4.2, from 2.1 to 4.2, from 2.2 to 4.2, from 2.3 to 4.2, from 2.4 to 4.2, from 2.5 to 4.2, from 2.6 to 4.2, from 2.7 to 4.2, from 2.8 to 4.2, from 2.9 to 4.2, from 3 to 4.2, from 3.1 to 4.2, from 3.2 to 4.2, from 3.3 to 4.2, from 3.4 to 4.2, from 3.5 to 4.2, from 3.6 to 4.2, from 3.7 to 4.2, from 3.8 to 4.2, from 3.9 to 4.2, from 4 to 4.2, from 1 to 4, from 1.1 to 4, from 1.2 to 4, from 1.3 to 4, from 1.4 to 4, from 1.5 to 4, from 1.6 to 4, from 1.7 to 4, from 1.8 to 4, from 1.9 to 4, from 2 to 4, from 2.1 to 4, from 2.2 to 4, from 2.3 to 4, from 2.4 to 4, from 2.5 to 4, from 2.6 to 4, from 2.7 to 4, from 2.8 to 4, from 2.9 to 4, from 3 to 4, from 3.1 to 4, from 3.2 to 4, from 3.3 to 4, from 3.4 to 4, from 3.5 to 4, from 3.6 to 4, from 3.7 to 4, from 3.8 to 4, from 3.9 to 4, from 1 to 3.9, from 1.1 to 3.9, from 1.2 to 3.9, from 1.3 to 3.9, from 1.4 to 3.9, from 1.5 to 3.9, from 1.6 to 3.9, from 1.7 to 3.9, from 1.8 to 3.9, from 1.9 to 3.9, from 2 to 3.9, from 2.1 to 3.9, from 2.2 to 3.9, from 2.3 to 3.9, from 2.4 to 3.9, from 2.5 to 3.9, from 2.6 to 3.9, from 2.7 to 3.9, from 2.8 to 3.9, from 2.9 to 3.9, from 3 to 3.9, from 3.1 to 3.9, from 3.2 to 3.9, from 3.3 to 3.9, from 3.4 to 3.9, from 3.5 to 3.9, from 3.6 to 3.9, from 3.7 to 3.9, from 3.8 to 3.9, from 1 to 3.8, from 1.1 to 3.8, from 1.2 to 3.8, from 1.3 to 3.8, from 1.4 to 3.8, from 1.5 to 3.8, from 1.6 to 3.8, from 1.7 to 3.8, from 1.8 to 3.8, from 1.9 to 3.8, from 2 to 3.8, from 2.1 to 3.8, from 2.2 to 3.8, from 2.3 to 3.8, from 2.4 to 3.8, from 2.5 to 3.8, from 2.6 to 3.8, from 2.7 to 3.8, from 2.8 to 3.8, from 2.9 to 3.8, from 3 to 3.8, from 3.1 to 3.8, from 3.2 to 3.8, from 3.3 to 3.8, from 3.4 to 3.8, from 3.5 to 3.8, from 3.6 to 3.8, from 3.7 to 3.8, from 1 to 3.7, from 1.1 to 3.7, from 1.2 to 3.7, from 1.3 to 3.7, from 1.4 to 3.7, from 1.5 to 3.7, from 1.6 to 3.7, from 1.7 to 3.7, from 1.8 to 3.7, from 1.9 to 3.7, from 2 to 3.7, from 2.1 to 3.7, from 2.2 to 3.7, from 2.3 to 3.7, from 2.4 to 3.7, from 2.5 to 3.7, from 2.6 to 3.7, from 2.7 to 3.7, from 2.8 to 3.7, from 2.9 to 3.7, from 3 to 3.7, from 3.1 to 3.7, from 3.2 to 3.7, from 3.3 to 3.7, from 3.4 to 3.7, from 3.5 to 3.7, from 3.6 to 3.7, from 1 to 3.6, from 1.1 to 3.6, from 1.2 to 3.6, from 1.3 to 3.6, from 1.4 to 3.6, from 1.5 to 3.6, from 1.6 to 3.6, from 1.7 to 3.6, from 1.8 to 3.6, from 1.9 to 3.6, from 2 to 3.6, from 2.1 to 3.6, from 2.2 to 3.6, from 2.3 to 3.6, from 2.4 to 3.6, from 2.5 to 3.6, from 2.6 to 3.6, from 2.7 to 3.6, from 2.8 to 3.6, from 2.9 to 3.6, from 3 to 3.6, from 3.1 to 3.6, from 3.2 to 3.6, from 3.3 to 3.6, from 3.4 to 3.6, from 3.5 to 3.6, from 1 to 3.5, from 1.1 to 3.5, from 1.2 to 3.5, from 1.3 to 3.5, from 1.4 to 3.5, from 1.5 to 3.5, from 1.6 to 3.5, from 1.7 to 3.5, from 1.8 to 3.5, from 1.9 to 3.5, from 2 to 3.5, from 2.1 to 3.5, from 2.2 to 3.5, from 2.3 to 3.5, from 2.4 to 3.5, from 2.5 to 3.5, from 2.6 to 3.5, from 2.7 to 3.5, from 2.8 to 3.5, from 2.9 to 3.5, from 3 to 3.5, from 3.1 to 3.5, from 3.2 to 3.5, from 3.3 to 3.5, from 3.4 to 3.5, from 1 to 3.4, from 1.1 to 3.4, from 1.2 to 3.4, from 1.3 to 3.4, from 1.4 to 3.4, from 1.5 to 3.4, from 1.6 to 3.4, from 1.7 to 3.4, from 1.8 to 3.4, from 1.9 to 3.4, from 2 to 3.4, from 2.1 to 3.4, from 2.2 to 3.4, from 2.3 to 3.4, from 2.4 to 3.4, from 2.5 to 3.4, from 2.6 to 3.4, from 2.7 to 3.4, from 2.8 to 3.4, from 2.9 to 3.4, from 3 to 3.4, from 3.1 to 3.4, from 3.2 to 3.4, from 3.3 to 3.4, from 1 to 3.3, from 1.1 to 3.3, from 1.2 to 3.3, from 1.3 to 3.3, from 1.4 to 3.3, from 1.5 to 3.3, from 1.6 to 3.3, from 1.7 to 3.3, from 1.8 to 3.3, from 1.9 to 3.3, from 2 to 3.3, from 2.1 to 3.3, from 2.2 to 3.3, from 2.3 to 3.3, from 2.4 to 3.3, from 2.5 to 3.3, from 2.6 to 3.3, from 2.7 to 3.3, from 2.8 to 3.3, from 2.9 to 3.3, from 3 to 3.3, from 3.1 to 3.3, from 3.2 to 3.3, from 1 to 3.2, from 1.1 to 3.2, from 1.2 to 3.2, from 1.3 to 3.2, from 1.4 to 3.2, from 1.5 to 3.2, from 1.6 to 3.2, from 1.7 to 3.2, from 1.8 to 3.2, from 1.9 to 3.2, from 2 to 3.2, from 2.1 to 3.2, from 2.2 to 3.2, from 2.3 to 3.2, from 2.4 to 3.2, from 2.5 to 3.2, from 2.6 to 3.2, from 2.7 to 3.2, from 2.8 to 3.2, from 2.9 to 3.2, from 3 to 3.2, from 3.1 to 3.2, from 1 to 3.1, from 1.1 to 3.1, from 1.2 to 3.1, from 1.3 to 3.1, from 1.4 to 3.1, from 1.5 to 3.1, from 1.6 to 3.1, from 1.7 to 3.1, from 1.8 to 3.1, from 1.9 to 3.1, from 2 to 3.1, from 2.1 to 3.1, from 2.2 to 3.1, from 2.3 to 3.1, from 2.4 to 3.1, from 2.5 to 3.1, from 2.6 to 3.1, from 2.7 to 3.1, from 2.8 to 3.1, from 2.9 to 3.1, from 3 to 3.1, from 1 to 3, from 1.1 to 3, from 1.2 to 3, from 1.3 to 3, from 1.4 to 3, from 1.5 to 3, from 1.6 to 3, from 1.7 to 3, from 1.8 to 3, from 1.9 to 3, from 2 to 3, from 2.1 to 3, from 2.2 to 3, from 2.3 to 3, from 2.4 to 3, from 2.5 to 3, from 2.6 to 3, from 2.7 to 3, from 2.8 to 3, from 2.9 to 3, from 1 to 2.9, from 1.1 to 2.9, from 1.2 to 2.9, from 1.3 to 2.9, from 1.4 to 2.9, from 1.5 to 2.9, from 1.6 to 2.9, from 1.7 to 2.9, from 1.8 to 2.9, from 1.9 to 2.9, from 2 to 2.9, from 2.1 to 2.9, from 2.2 to 2.9, from 2.3 to 2.9, from 2.4 to 2.9, from 2.5 to 2.9, from 2.6 to 2.9, from 2.7 to 2.9, from 2.8 to 2.9, from 1 to 2.8, from 1.1 to 2.8, from 1.2 to 2.8, from 1.3 to 2.8, from 1.4 to 2.8, from 1.5 to 2.8, from 1.6 to 2.8, from 1.7 to 2.8, from 1.8 to 2.8, from 1.9 to 2.8, from 2 to 2.8, from 2.1 to 2.8, from 2.2 to 2.8, from 2.3 to 2.8, from 2.4 to 2.8, from 2.5 to 2.8, from 2.6 to 2.8, from 2.7 to 2.8, from 1 to 2.7, from 1.1 to 2.7, from 1.2 to 2.7, from 1.3 to 2.7, from 1.4 to 2.7, from 1.5 to 2.7, from 1.6 to 2.7, from 1.7 to 2.7, from 1.8 to 2.7, from 1.9 to 2.7, from 2 to 2.7, from 2.1 to 2.7, from 2.2 to 2.7, from 2.3 to 2.7, from 2.4 to 2.7, from 2.5 to 2.7, from 2.6 to 2.7, from 1 to 2.6, from 1.1 to 2.6, from 1.2 to 2.6, from 1.3 to 2.6, from 1.4 to 2.6, from 1.5 to 2.6, from 1.6 to 2.6, from 1.7 to 2.6, from 1.8 to 2.6, from 1.9 to 2.6, from 2 to 2.6, from 2.1 to 2.6, from 2.2 to 2.6, from 2.3 to 2.6, from 2.4 to 2.6, from 2.5 to 2.6, from 1 to 2.5, from 1.1 to 2.5, from 1.2 to 2.5, from 1.3 to 2.5, from 1.4 to 2.5, from 1.5 to 2.5, from 1.6 to 2.5, from 1.7 to 2.5, from 1.8 to 2.5, from 1.9 to 2.5, from 2 to 2.5, from 2.1 to 2.5, from 2.2 to 2.5, from 2.3 to 2.5, from 2.4 to 2.5, from 1 to 2.4, from 1.1 to 2.4, from 1.2 to 2.4, from 1.3 to 2.4, from 1.4 to 2.4, from 1.5 to 2.4, from 1.6 to 2.4, from 1.7 to 2.4, from 1.8 to 2.4, from 1.9 to 2.4, from 2 to 2.4, from 2.1 to 2.4, from 2.2 to 2.4, from 2.3 to 2.4, from 1 to 2.3, from 1.1 to 2.3, from 1.2 to 2.3, from 1.3 to 2.3, from 1.4 to 2.3, from 1.5 to 2.3, from 1.6 to 2.3, from 1.7 to 2.3, from 1.8 to 2.3, from 1.9 to 2.3, from 2 to 2.3, from 2.1 to 2.3, from 2.2 to 2.3, from 1 to 2.2, from 1.1 to 2.2, from 1.2 to 2.2, from 1.3 to 2.2, from 1.4 to 2.2, from 1.5 to 2.2, from 1.6 to 2.2, from 1.7 to 2.2, from 1.8 to 2.2, from 1.9 to 2.2, from 2 to 2.2, from 2.1 to 2.2, from 1 to 2.1, from 1.1 to 2.1, from 1.2 to 2.1, from 1.3 to 2.1, from 1.4 to 2.1, from 1.5 to 2.1, from 1.6 to 2.1, from 1.7 to 2.1, from 1.8 to 2.1, from 1.9 to 2.1, from 2 to 2.1, from 1 to 2, from 1.1 to 2, from 1.2 to 2, from 1.3 to 2, from 1.4 to 2, from 1.5 to 2, from 1.6 to 2, from 1.7 to 2, from 1.8 to 2, from 1.9 to 2, from 1 to 1.9, from 1.1 to 1.9, from 1.2 to 1.9, from 1.3 to 1.9, from 1.4 to 1.9, from 1.5 to 1.9, from 1.6 to 1.9, from 1.7 to 1.9, from 1.8 to 1.9, from 1 to 1.8, from 1.1 to 1.8, from 1.2 to 1.8, from 1.3 to 1.8, from 1.4 to 1.8, from 1.5 to 1.8, from 1.6 to 1.8, from 1.7 to 1.8, from 1 to 1.7, from 1.1 to 1.7, from 1.2 to 1.7, from 1.3 to 1.7, from 1.4 to 1.7, from 1.5 to 1.7, from 1.6 to 1.7, from 1 to 1.6, from 1.1 to 1.6, from 1.2 to 1.6, from 1.3 to 1.6, from 1.4 to 1.6, from 1.5 to 1.6, from 1 to 1.5, from 1.1 to 1.5, from 1.2 to 1.5, from 1.3 to 1.5, from 1.4 to 1.5, from 1 to 1.4, from 1.1 to 1.4, from 1.2 to 1.4, from 1.3 to 1.4, from 1 to 1.3, from 1.1 to 1.3, from 1.2 to 1.3, from 1 to 1.2, from 1.1 to 1.3, or from 1 to 1.1) times the half-life of the radical initiator.

Also provided are embodiments of the processes to produce the radically-functionalized PIBSA product described above, wherein the process comprises reacting polyisobutylene (such as high-vinylidene polyisobutylene) with a radical initiator and an ethylenically unsaturated acylating agent at a temperature of from 150 °C to 225 °C to produce the radically-functionalized PIBSA product, wherein the polyisobutylene is brought to the reaction temperature in a continuous reactor, prior to commencing continuous addition of the radical initiator to the polyisobutylene in the continuous reactor; and wherein the ethylenically unsaturated acylating agent is added to the continuous reactor: (i) as a mixture with the polyisobutylene; (ii) as a mixture with the radical initiator; and/or (iii) continuously and separately from either the polyisobutylene or the radical initiator.

An illustrative, non-limiting embodiment of the processes to produce the radically-functionalized PIBSA product described herein may include feeding polyisobutylene into a two- or three-stage reactor system at 170 °C, with each stage including an addition of maleic anhydride acylating agent and di-tertbutyl peroxide initiator to the polyisobutylene flowing through the reactor system. With the half-life of di-tertbutyl peroxide being about 6 minutes, each stage is allowed to react for about 20 minutes to allow most of the initiator to be consumed before adding another dose. After two or three stages, in about 40 to 60 minutes, the reaction is complete and the desired product has been achieved.

### Amine Reactant

The radically-functionalized PIBSA product may be further reacted with an amine containing compound capable of reacting with the acylating group of the PIBSA to form succinimides or succinamides, which may be used as dispersants in various compositions, including lubricant compositions.

The succinimide and succinamide dispersant may be derived from an aliphatic polyamine, or mixtures thereof. The aliphatic polyamine may be aliphatic polyamine such as an ethylenepolyamine, a propylenepolyamine, a butylenepolyamine, or mixtures thereof. In certain embodiments the aliphatic polyamine may be ethylenepolyamine. In certain embodiments the aliphatic polyamine may be chosen from ethylenediamine, diethylenetriamine, triethylenetetramine, tetraethylenepentamine, pentaethylenehexamine, polyamine still bottoms, and mixtures thereof.

The amine reactants may be those described in the art as “polyamine bottoms.” Polyamine bottoms are polyamine mixtures obtained as the residue from stripping complex mixtures of alkylene, usually ethylene, polyamines which complex mixtures include cyclic condensation products such as piperazines. These complex alkylene polyamine mixtures are typically those produced by the reaction of alkylene chloride, usually alkylene dichlorides, with ammonia or reaction of an ethylene imine with a ring opening reagent such as water or ammonia. In general, alkylene polyamine bottoms can be characterized as having less than 2%, usually less than 1% by weight material boiling below 200 °C.

Suitable ethylene polyamine bottoms are those designated as “E-100,” available from Dow Chemical Co., Freeport Tex., USA. This material has nominal specifications of specific gravity at 15.6 °C of 1.0168; % N=33.15, kinematic viscosity at 40 °C.=121 mm²/s (centistokes, cSt). Gas chromatography shows it contains about 0.93% light ends (most probably diethylene triamine), 0.72% triethylenetetramine, tetraethylenepentamine and 76.61% pentaethylene hexamine and higher (all by weight). Another example of polyethylene bottoms is a product sold by Union Carbide Chemicals as HPA-X, having equivalent weight (per N)=40.5.

The polyamines contain at least one >N—H group per molecule. These polyamines bottoms can be reacted solely with the acylating agent or they can be used with other amines, polyamines or mixtures thereof, provided that the major amount, on an equivalent N—H basis, is alkylene polyamine bottoms.

The acylated amine may comprise at least one member of the group consisting of amide, imide and salt. Often the acylated amine is a mixture of two or more of these. The acylated amine may possess a base number arising from the presence of the amine.

Often the total base number on a neat chemical basis (that is, correcting for the presence of any diluent oil) is 17 or 20 to 35, more often 20 or 24 to 30. In certain embodiments it can be 17 to 20. When the acylated amine is prepared from a low chlorine containing acylating agent the chlorine content of the acylated amine also is correspondingly low. The chlorine content, on a neat chemical basis, may be up to 0.6% e.g., 0.01% to 0.6% or to 0.4% or to 0.2%.

Reactions to prepare the hydrocarbyl substituted acylating agent are usually conducted in the substantial absence of diluent, although a substantially inert, normally liquid diluent such as mineral oil or hydrocarbon solvent may be used. The reaction of the hydrocarbyl substituted acylating agent with the amine may also be conducted neat or in the presence of a substantially inert, normally liquid diluent. Typically, if a diluent is used, it comprises mineral oil which remains in the product. If volatile diluents are used, it is usually necessary to remove the diluent by techniques such as distillation such that the resulting product has an acceptable flash point, i.e., is not unacceptably flammable. When a diluent is present in the acylated amine, it is usually present in an amount of 30 to 70 parts per 100 parts of acylated amine. As noted hereinabove, polyolefin remaining unreacted from the reaction forming the hydrocarbon group substituted acylating agent is deemed to be part of the diluent. The following examples illustrate hydrocarbyl substituted carboxylic acylating agents and acylated amines described herein. All temperatures are in degrees Celsius, parts and percentages are by weight and filtrations are conducted with a diatomaceous earth filter aid.

### Alcohol Reactant

Another class of dispersants which may be prepared from the radically-functionalized PIBSA product are succinate esters. These materials are similar to the above-described succinimides or succinimides, except that they may be seen as having been prepared by reaction of the radically-functionalized PIBSA product with a polyhydric aliphatic alcohol such as glycerol, pentaerythritol, or sorbitol. Aromatic succinate esters may also be prepared. In some instances, these ester type dispersants can be post-treated with an amine such as a poly(ethyleneamine).

### Post-Treatment

The succinimides, succinamides and succinate esters described above can be post-treated. Post-treated dispersants are generally obtained by reacting succinimides, succiamides, or succinate esters with reagents such as urea, thiourea, carbon disulfide, aldehydes, ketones, carboxylic acids, hydrocarbon-substituted succinic anhydrides, nitriles, epoxides, boron compounds such as boric acid, phosphorus compounds such as phosphorus acids or anhydrides, 2,5-dimercaptothiadiazole (DMTD), or an aromatic diacid having acid groups in 1,3 or 1,4 positions on a benzene ring (such as terepthahlic acid).

Borated dispersants are generally obtained by reacting the succinimides, succinamides, or succinate esters with a boron compound reagent, such as boric acid (to give “borated dispersants”). The borated dispersant may be further functionalized with a sulfur or phosphorus moiety. The dispersant component in the borated dispersant may be a mixture of multiple dispersants which may be of different types; optionally at least one may be a succinimide dispersant. In certain embodiments the borated dispersant may be a borated polyisobutylene succinimide dispersant, in which the polyisobutylene portion thereof may have a number average molecular weight of 750 to 2200, or 750 to 1350, or 750 to 1150. The borated dispersant(s) may be prepared in such a way to have a N:CO ratio of 0.9:1 to 1.6:1, or 0.95:1 to 1.5:1, or 1:1 to 1.4:1. The amount of borated dispersant in the compositions, may be, for instance, 0.05 to 2.0 percent by weight. In other embodiments, the amount is 0.1 to 1.0 percent or 0.15 to 0.75 percent of the final blended fluid formulation. In a concentrate, the amounts will be proportionately higher.

In certain embodiments, the succinimides or succinamides of the present technology may be a borated succinimide or succinamide prepared by heating together: (i) a dispersant substrate; (ii) 2,5-dimercapto-1,3,4-thiadiazole or a hydrocarbylsubstituted 2,5-di-mercapto-1,3,4-thiadiazole, or oligomers thereof; (iii) a borating agent; and (iv) optionally a dicarboxylic acid of an aromatic compound chosen from 1,3 diacids and 1,4 diacids; and/or (v) optionally a phosphorus acid compound, said heating being sufficient to provide a product of (i), (ii), (iii) and optionally (iv) or (v), which is soluble in an oil of lubricating viscosity.

### Lubricant Compositions

The reaction products described above may be useful in various compositions, such as lubricant compositions. In certain embodiments, a lubricant composition may comprise an oil of lubricating viscosity and the reaction product comprising at least one of a succinimide, a succinamide, or a succinate ester. In certain embodiments, the lubricant composition may comprise at least one additional additive. In certain embodiments, the at least one additional additive may comprise at least one of detergents, dispersants, friction modifiers, antiwear agents, extreme pressure agents, corrosion inhibitors, viscosity modifiers, antifoam agents, demulsifiers, or seal swell agents.

In certain embodiments, provided is a method of lubricating a mechanical device comprising supplying the lubricant composition to the mechanical device. In certain embodiments, the mechanical device is the crankcase of an internal combustion engine, a gear, a gearbox, an axle gear, or a transmission. In certain embodiments, the internal combustion engine is a spark ignited engine, or a compression ignited engine. In certain embodiments, the gear, the gearbox, or the axle gear comprises a differential, or a limited slip differential. In certain embodiments, the transmission is an automatic transmission. In certain embodiments, the automatic transmission comprises a continuously variable transmission, an infinitely variable transmission, a toroidal transmission, a continuously slipping torque converted clutch, a stepped automatic transmission, or a dual clutch transmission. In certain embodiments, the transmission is a manual transmission. In certain embodiments, the manual transmission is a farm tractor transmission.

In certain embodiments the technology provides lubricating compositions that can be employed in internal combustion engines. The internal combustion engine may be spark ignition or compression ignition. The internal combustion engine may be a 2-stroke or 4-stroke engine. The internal combustion engine may be a passenger car engine, a light duty diesel engine, a heavy duty diesel engine, a motorcycle engine, or a 2-stroke or 4-stroke marine diesel engine. Typically, the internal combustion engine may be a passenger car engine, or a heavy duty diesel internal combustion engine.

In certain embodiments, provided is a lubricant composition comprising: an oil of lubricating viscosity; 0.01 wt % to 12 wt %, or 0.05 to 8 wt %, or 0.1 to 6 wt %, or 0.1 to 2 wt % of the succinimide, succinamide, and/or succinate ester of the present technology; 0.01 wt % to 10 wt %, or 0.1 wt % to 10 wt %, or 0.2 wt % to 8 wt %, or 0.2 wt % to 3 wt % of an overbased detergent; and a phosphorus-containing antiwear agent chosen from (i) a non-ionic phosphorus compound, which may be a hydrocarbyl phosphite; (ii) an amine salt of a phosphorus compound, (iii) zinc dialkyldithiophosphate. or (iv) Zinc dialkyldithiophosphates.

In certain embodiments, provided is a method of lubricating the crankcase of an internal combustion engine comprising supplying to the crankcase of the internal combustion engine a lubricant composition comprising: an oil of lubricating viscosity; 0.01 wt % to 12 wt %, or 0.05 to 8 wt %, or 0.1 to 6 wt %, or 1 to 5 wt % of the succinimide, succinamide, and/or succinate ester of the present technology; 0.01 wt % to 10 wt %, or 0.1 wt % to 10 wt %, or 0.2 wt % to 8 wt %, or 0.2 wt % to 3 wt % of an overbased detergent; and a phosphorus-containing antiwear agent chosen from (i) a non-ionic phosphorus compound, which may be a hydrocarbyl phosphite; (ii) an amine salt of a phosphorus compound, (iii) zinc dialkyldithiophosphate. or (iv) Zinc dialkyldithiophosphates.

In certain embodiments, provided is a lubricating compositions that can be employed in a gear, gearbox, or axle gear. The gearbox may have a differential, or a limited slip differential.

In certain embodiments, provided is a lubricant composition comprising: 0.01 wt % to 12 wt %, or 0.05 to 8 wt %, or 0.1 to 6 wt %, or 0.1 to 2 wt % of the succinimide, succinamide, and/or succinate ester of the present technology; an olefin sulfide (for example, present at 0.1 to 7 wt %, or 0.2 to 6 wt %, or 0.5 to 5 wt %, or 1 to 5 wt %); and a thiadiazole, (for example, present at 0.1 to 0.5 wt %, or 0.15 to 0.4 wt %, or 0.15 to 0.35 wt %).

In certain embodiments, provided is a method of lubricating a gear or gearbox or axle gear comprising a differential, a limited slip differential by supplying to the gear or gearbox, a lubricant composition comprising: 0.01 wt % to 12 wt %, or 0.05 to 8 wt %, or 0.1 to 6 wt %, or 0.1 to 2 wt % of the succinimide, succinamide, and/or succinate ester of the present technology; an olefin sulfide (for example, present at 0.1 to 7 wt %, or 0.2 to 6 wt %, or 0.5 to 5 wt %, or 1 to 5 wt %); and a thiadiazole, (for example, present at 0.1 to 0.5 wt %, or 0.15 to 0.4 wt %, or 0.15 to 0.35 wt %).

In certain embodiments, provided is a lubricating compositions that can be employed in Automatic Transmission. The automatic transmission includes continuously variable transmissions (CVT), infinitely variable transmissions (IVT), Toroidal transmissions, continuously slipping torque converted clutches (CSTCC), stepped automatic transmissions or dual clutch transmissions (DCT).

In certain embodiments, provided is a lubricant composition comprising: an oil of lubricating viscosity; 0.01 wt % to 5 wt %, or 0.05 to 3 wt %, or 0.1 to 3 wt %, or 0.1 to 2 wt % of the succinimide, succinamide, and/or succinate ester of the present technology; a phosphorus-containing antiwear agent chosen from (i) a non-ionic phosphorus compound, which may be a hydrocarbyl phosphite, or (ii) an amine salt of a phosphorus compound; a calcium-containing detergent, for example, present in an amount to deliver 110 to 700 ppm, 130 to 600 ppm, 150 to 500 ppm, or 160 to 400 ppm calcium; and optionally a friction modifier, for example, present at 0 to 4 wt %, or 0.1 to 4 wt %, or 0.2 to 3 wt %,or 0.3 to 3 wt %, or 0.25 to 2.5 wt %. In certain embodiments, the friction modifier is present. In certain embodiments, the friction modifier is absent.

In certain embodiments, provided is a method of lubricating an automatic transmission comprising supplying to the automatic transmission a lubricant composition comprising: an oil of lubricating viscosity; 0.01 to 5 wt %, or 0.05 to 3 wt %, or 0.1 to 3 wt %, or 0.1 to 2 wt % of the succinimide, succinamide, and/or succinate ester of the present technology; a phosphorus-containing antiwear agent chosen from (i) a non-ionic phosphorus compound, which may be a hydrocarbyl phosphite, or (ii) an amine salt of a phosphorus compound; and a calcium-containing detergent, for example, present in an amount to deliver 110 to 700 ppm, 130 to 600 ppm, 150 to 500 ppm or 160 to 400 ppm calcium.

In certain embodiments, provided is a lubricant composition comprising: an oil of lubricating viscosity; 0.01 to 5 wt %, or 0.05 to 3 wt %, or 0.1 to 3 wt %, or 0.1 to 2 wt % of the succinimide, succinamide, and/or succinate ester of the present technology; 0.05 to 1 wt % of a thiadiazole; 0.1 wt % to 5 wt % of a dispersant other than the succinimides, succinamides or succinate esters of the present technology; and 0.1 wt % to 4 wt % of a detergent.

In certain embodiments, provided is a method of lubricating a manual transmission by supplying to the manual transmission a lubricant composition comprising: an oil of lubricating viscosity; 0.01 to 5 wt %, or 0.05 to 3 wt %, or 0.1 to 3 wt %, or 0.1 to 2 wt % of the succinimide, succinamide, and/or succinate ester of the present technology; 0.05 to 1 wt % of a thiadiazole; 0.1 wt % to 5 wt % of a dispersant other than the succinimides, succinamides or succinate esters of the present technology; and 0.1 wt % to 4 wt % of a detergent.

### Oils of Lubricating Viscosity

The succinimides, succinamides and succinate esters described above may further be used in lubricating oils to provide at least one of antiwear performance, friction modification (particularly for enhancing fuel economy), detergent performance (particularly deposit control or varnish control), dispersancy (particularly soot control, or sludge control), or corrosion control.

The lubricant composition also comprises an oil of lubricating viscosity. Such oils include natural and synthetic oils, oil derived from hydrocracking, hydrogenation, and hydrofinishing, unrefined, refined, re-refined oils or mixtures thereof. A more detailed description of unrefined, refined and re-refined oils is provided in International Publication WO2008/147704, paragraphs [0054] to [0056](a similar disclosure is provided in US Patent Application 2010/197536, see [0072] to [0073]). A more detailed description of natural and synthetic lubricating oils is described in paragraphs [0058] to [0059] respectively of WO2008/147704 (a similar disclosure is provided in US Patent Application 2010/197536, see [0075] to [0076]). Synthetic oils may also be produced by Fischer-Tropsch reactions and typically may be hydroisomerised Fischer-Tropsch hydrocarbons or waxes. In certain embodiments oils may be prepared by a Fischer-Tropsch gas-to-liquid synthetic procedure as well as other gasto-liquid oils.

Oils of lubricating viscosity may also be defined as specified in April 2008 version of “Appendix E—API Base Oil Interchangeability Guidelines for Passenger Car Motor Oils and Diesel Engine Oils”, section 1.3 Sub-heading 1.3. “Base Stock Categories”. The API Guidelines are also summarised in U.S. Pat. No. 7,285,516 (see column 11, line 64 to column 12, line 10). In certain embodiments the oil of lubricating viscosity may be an API Group II, Group III, Group IV oil, or mixtures thereof.

The amount of the oil of lubricating viscosity present is typically the balance remaining after subtracting from 100 wt % the sum of the amount of the compound and the other performance additives.

The lubricant composition may be in the form of a concentrate and/or a fully formulated lubricant. If the lubricant composition is in the form of a concentrate (which may be combined with additional oil to form, in whole or in part, a finished lubricant), the ratio of the of these additives to the oil of lubricating viscosity and/or to diluent oil include the ranges of 1:99 to 99:1 by weight, or 80:20 to 10:90 by weight.

### Detergents

In certain embodiments, the lubricant composition comprises, in addition to the succinimides, succinamides and succinate of the present technology, an overbased metal-containing detergent, or mixtures thereof.

Overbased detergents are known in the art. Overbased materials, otherwise referred to as overbased or superbased salts, are generally single phase, homogeneous systems characterized by a metal content in excess of that which would be present for neutralization according to the stoichiometry of the metal and the particular acidic organic compound reacted with the metal. The overbased materials are prepared by reacting an acidic material (typically an inorganic acid or lower carboxylic acid, typically carbon dioxide) with a mixture comprising an acidic organic compound, a reaction medium comprising at least one inert, organic solvent (mineral oil, naphtha, toluene, xylene, etc.) for said acidic organic material, a stoichiometric excess of a metal base, and a promoter such as a calcium chloride, acetic acid, phenol or alcohol. The acidic organic material will normally have a sufficient number of carbon atoms to provide a degree of solubility in oil. The amount of “excess” metal (stoichiometrically) is commonly expressed in terms of metal ratio. The term “metal ratio” is the ratio of the total equivalents of the metal to the equivalents of the acidic organic compound. A neutral metal salt has a metal ratio of one. A salt having 4.5 times as much metal as present in a normal salt will have metal excess of 3.5 equivalents, or a ratio of 4.5. The term “metal ratio” is also explained in standard textbook entitled “Chemistry and Technology of Lubricants”, Third Edition, Edited by R. M. Mortier and S. T. Orszulik, Copyright 2010, page 219, sub-heading 7.25.

The overbased metal-containing detergent may be chosen from non-sulfur-containing phenates, sulfur-containing phenates, sulfonates, salixarates, salicylates, carboxylates, and mixtures thereof, or borated equivalents thereof. The overbased detergent may be borated with a borating agent such as boric acid.

The overbased detergent may be non-sulfur containing phenates, sulfur containing phenates, sulfonates, or mixtures thereof.

In one particular embodiment the lubricant composition comprises and overbased sulfonate. Overbased sulfonates typically have a total base number of 250 to 600, or 300 to 500 (on an oil free basis). Overbased detergents are known in the art. In certain embodiments the sulfonate detergent may be a predominantly linear alkylbenzene sulfonate detergent having a metal ratio of at least 8 as is described in paragraphs [0026] to [0037] of US Patent Application 2005065045 (and granted as U.S. Pat. No. 7,407,919). Linear alkyl benzenes may have the benzene ring attached anywhere on the linear chain, usually at the 2, 3, or 4 position, or mixtures thereof. The predominantly linear alkylbenzene sulfonate detergent may be particularly useful for assisting in improving fuel economy. In certain embodiments the sulfonate detergent may be a metal salt of one or more oil-soluble alkyl toluene sulfonate compounds as disclosed in paragraphs [0046] to [0053] of US Patent Application 2008/0119378.

In certain embodiments, the overbased metal detergent comprises an overbased calcium sulfonate. The calcium sulfonate detergent may have a metal ratio of 18 to 40 and a TBN of 300 to 500, or 325 to 425.

The overbased detergent may also include “hybrid” detergents formed with mixed surfactant systems including phenate and/or sulfonate components, e.g., phenate/salicylates, sulfonate/phenates, sulfonate/salicylates, sulfonates/phenates/salicylates, as described; for example, in U.S. Pat. Nos. 6,429,178; 6,429,179; 6,153,565; and 6,281,179. Where, for example, a hybrid sulfonate/phenate detergent is employed, the hybrid detergent would be considered equivalent to amounts of distinct phenate and sulfonate detergents introducing like amounts of phenate and sulfonate soaps, respectively.

The overbased detergent may have an alkali metal, an alkaline earth metal, or zinc counter ion. In certain embodiments the metal may be sodium, calcium, barium, or magnesium. Typically, other detergent may be sodium, calcium, or magnesium containing detergent (typically, calcium, or magnesium containing detergent).

The overbased detergent may typically be an overbased detergent of sodium, calcium or magnesium salt of the phenates, sulfur-containing phenates, salixarates and salicylates. Overbased phenates and salicylates typically have a total base number of 180 to 450 TBN (on an oil free basis).

Phenate detergents are typically derived from p-hydrocarbyl phenols. Alkylphenols of this type may be coupled with sulfur and overbased, coupled with aldehyde and overbased, or carboxylated to form salicylate detergents. Suitable alkylphenols include those alkylated with oligomers of propylene, i.e. tetrapropenylphenol (i.e. p-dodecylphenol or PDDP) and pentapropenylphenol. Other suitable alkylphenols include those alkylated with alpha-olefins, isomerized alpha-olefins, and polyolefins like polyisobutylene. In certain embodiments, the lubricating composition comprises less than 0.2 wt %, or less than 0.1 wt %, or even less than 0.05 wt % of a phenate detergent derived from PDDP. In certain embodiments, the lubricant composition comprises a phenate detergent that is not derived from PDDP.

The overbased detergent may be present at 0 wt % to 10 wt %, or 0.1 wt % to 10 wt %, or 0.2 wt % to 8 wt %, or 0.2 wt % to 3 wt %. For example, in a heavy duty diesel engine the detergent may be present at 2 wt % to 3 wt % of the lubricant composition. For a passenger car engine the detergent may be present at 0.2 wt % to 1 wt % of the lubricant composition. In certain embodiments, an engine oil lubricant composition comprises at least one overbased detergent with a metal ratio of at least 3, or at least 8, or at least 15.

In certain embodiments, the overbased detergent is a calcium-containing detergent. The calcium-containing detergent may be an overbased detergent, a non-overbased detergent, or mixtures thereof. Typically, the detergent may be overbased.

The preparation of the calcium-containing detergent is known in the art. Patents describing the preparation of overbased calcium-containing detergents include U.S. Pat. Nos. 2,501,731; 2,616,905; 2,616,911; 2,616,925; 2,777, 874; 3,256,186; 3,384,585; 3,365,396; 3,320,162; 3,318, 809; 3,488,284; and 3,629,109.

The calcium-containing detergent may be a non-overbased detergent (may also be referred to as a neutral detergent). The TBN of a non-overbased may be 20 to less than 200, or 30 to 100, or 35 to 50 mg KOH/g. The TBN of a non-overbased calcium-containing detergent may also be 20 to 175, or 30 to 100 mg KOH/g. When a non-overbased calcium-containing detergent is prepared from a strong acid such as a hydrocarbyl-substituted sulphonic acid, the TBN may be lower (for example, 0 to 50 mg KOH/g, or 10 to 20 mg KOH/g).

The calcium-containing detergent may be an overbased detergent, which may have a TBN of greater than 200 mg KOH/g (typically 250 to 600, or 300 to 500 mg KOH/g).

The calcium-containing detergent may be formed by the reaction of a basic calcium compound and an acidic detergent substrate. The acidic detergent substrate may include an alkyl phenol, an aldehyde-coupled alkyl phenol, a sulfurized alkyl phenol, an alkyl aromatic sulphonic acid (such as, alkyl naphthalene sulphonic acid, alkyl toluene sulphonic acid or alkyl benzene sulphonic acid), an aliphatic carboxylic acid, a calixarene, a salixarene, an alkyl salicylic acid, or mixtures thereof.

The metal basic compound is used to supply basicity to the detergent. The basic calcium compound is a compound of a hydroxide or oxide of the metal.

The oxides and/or hydroxides may be used alone or in combination. The oxides or hydroxides may be hydrated or dehydrated, although hydrated is typical. In certain embodiments, the basic calcium compound may be calcium hydroxide, which may be used alone or mixtures thereof with other metal basic compounds. Calcium hydroxide is often referred to as lime. In certain embodiments, the metal basic compound may be calcium oxide which may be used alone or mixtures thereof with other metal basic compounds.

Collectively, when the alkyl phenol, the aldehyde-coupled alkyl phenol, and the sulfurized alkyl phenol are used to prepare a calcium-containing detergent, the detergent may be referred to as a calcium phenate. The calcium phenate may be an alkyl phenate, an aldehyde-coupled alkyl phenate, a sulfurized alkyl phenate, or mixtures thereof.

The TBN of a calcium phenate may vary from less 200, or 30 to 175 typically 150 to 175) mg KOH/g for a neutral phenate to 200 or more to 500, or 210 to 400 (typically 230 to 270) mg KOH/g for an overbased phenate.

The alkyl group of a phenate (i.e., an alkyl phenate) may contain 4 to 80, or 6 to 45, or 8 to 20, or 9 to 15 carbon atoms.

In certain embodiments, the calcium-containing detergent may be a sulphonate, or mixtures thereof. The sulphonate may be prepared from a mono- or di-hydrocarbyl-substituted benzene (or naphthalene, indenyl, indanyl, or bicyclopentadienyl) sulphonic acid, wherein the hydrocarbyl group may contain 6 to 40, or 8 to 35 or 9 to 30 carbon atoms.

The hydrocarbyl group may be derived from polypropylene or a linear or branched alkyl group containing at least 10 carbon atoms. Examples of a suitable alkyl group include branched and/or linear decyl, undecyl, dodecyl, tridecyl, tetradecyl, pentadecyl, hexadecyl, heptadecyl, octadecyl, octadecenyl, nonodecyl, eicosyl, un-eicosyl, do-eicosyl, tri-eicosyl, tetra-eicosyl, penta-eicosyl, hexa-eicosyl or mixtures thereof.

In certain embodiments, the hydrocarbyl-substituted sulphonic acid may include polypropene benzenesulphonic acid and/or C₁₆-C₂₄ alkyl benzenesulphonic acid, or mixtures thereof.

In certain embodiments, a calcium sulphonate detergent may be a predominantly linear alkylbenzene sulphonate detergent having a metal ratio of at least 8 as is described in paragraphs [0026] to [0037] of US Patent Application 2005065045 (and granted as U.S. Pat. No. 7,407,919). In some embodiments, the linear alkyl group may be attached to the benzene ring anywhere along the linear chain of the alkyl group, but often in the 2, 3 or 4 position of the linear chain, and in some instances predominantly in the 2 position.

When neutral or slightly basic, a calcium sulphonate detergent may have TBN of less than 100, or less than 75, typically 20 to 50 mg KOH/g, or 0 to 20 mg KOH/g.

When overbased, a calcium sulphonate detergent may have a TBN greater than 200, or 300 to 550, or 350 to 450 mg KOH/g.

The detergent may be borated or non-borated.

Chemical structures for sulphonates, and phenates detergents are known to a person skilled in the art. The standard textbook entitled “Chemistry and Technology of Lubricants”, Third Edition, Edited by R. M. Mortier and S. T. Orszulik, Copyright 2010, pages 220 to 223 under the sub-heading 7.2.6 provide general disclosures of said detergents and their structures.

In certain embodiments, the calcium-containing detergent may be an overbased calcium sulphonate, an overbased calcium phenate, or mixtures thereof. Typically, the detergent may be an overbased calcium sulphonate.

In certain embodiments, the calcium-containing detergent may be in a mixture with a having zinc-, barium-, sodium-, or magnesium-containing detergent. The zinc-, barium-, sodium-, or magnesium-containing detergent is also well known in the art and described in the same references describing a calcium-containing detergent. The TBN and metal ratios may however, differ slightly. The zinc-, barium-, sodium-, or magnesium-containing detergent may be a phenate, a sulfur-containing phenate, sulphonate, salixarate or salicylate. Typically, a zinc-, barium-, sodium-, or magnesium-containing detergent may be a magnesium phenate, a magnesium sulfur-containing phenate, or a magnesium sulphonate.

### Dispersants

In certain embodiments, an engine oil lubricant composition comprising the succinimides, succinamides, or succinate esters of the present technology may further include a dispersant, or mixtures thereof. The dispersant may be chosen from a succinimide dispersant, a Mannich dispersant, a succinamide dispersant, a polyolefin succinic acid ester, amide, or ester-amide, or mixtures thereof.

In certain embodiments, an engine oil lubricant composition includes a dispersant or mixtures thereof. The dispersant may be present as a single dispersant. The dispersant may be present as a mixture of two or more (typically two or three) different dispersants, wherein at least one may be a succinimide dispersant.

The succinimide dispersant may be derived from an aliphatic polyamine, or mixtures thereof. The aliphatic polyamine may be aliphatic polyamine such as an ethylenepolyamine, a propylenepolyamine, a butylenepolyamine, or mixtures thereof. In certain embodiments the aliphatic polyamine may be ethylenepolyamine. In certain embodiments the aliphatic polyamine may be chosen from ethylenediamine, diethylenetriamine, triethylenetetramine, tetraethylenepentamine, pentaethylenehexamine, polyamine still bottoms, and mixtures thereof.

In certain embodiments, the dispersant may be a polyolefin succinic acid ester, amide, or ester-amide. For instance, a polyolefin succinic acid ester may be a polyisobutylene succinic acid ester of pentaerythritol, or mixtures thereof. A polyolefin succinic acid ester-amide may be a polyisobutylene succinic acid reacted with an alcohol (such as pentaerythritol) and an amine (such as a diamine, typically diethyleneamine).

The dispersant may be an N-substituted long chain alkenyl succinimide. An example of an N-substituted long chain alkenyl succinimide is polyisobutylene succinimide. Typically, the polyisobutylene from which polyisobutylene succinic anhydride may be derived has a number average molecular weight of 350 to 5000, or 550 to 3000 or 750 to 2500. Succinimide dispersants and their preparation are disclosed, for instance in U.S. Pat. Nos. 3,172,892, 3,219, 666, 3,316,177, 3,340,281, 3,351,552, 3,381,022, 3,433,744, 3,444,170, 3,467,668, 3,501,405, 3,542,680, 3,576,743, 3,632,511, 4,234,435, Re 26,433, and U.S. Pat. Nos. 6,165,235, 7,238,650 and EP Patent Application 0 355 895 A.

The dispersants may also be post-treated by conventional methods by a reaction with any of a variety of agents. Among these are boron compounds (such as boric acid), urea, thiourea, dimercaptothiadiazoles, carbon disulfide, aldehydes, and ketones, carboxylic acids such as terephthalic acid, hydrocarbon-substituted succm1c anhydrides, maleic anhydride, nitriles, epoxides, and phosphorus compounds. In certain embodiments the post-treated dispersant is borated. In certain embodiments the post-treated dispersant may be reacted with dimercaptothiadiazoles. In certain embodiments the post-treated dispersant may be reacted with phosphoric or phosphorous acid. In certain embodiments the post-treated dispersant may be reacted with terephthalic acid and boric acid (as described in US Patent Application US2009/0054278.

In certain embodiments, the dispersant may be borated or non-borated. Typically a borated dispersant may be a succinimide dispersant. In certain embodiments, the ashless dispersant may be boron-containing, i.e., has incorporated boron and delivers said boron to the lubricant composition. The boron-containing dispersant may be present in an amount to deliver at least 25 ppm boron, at least 50 ppm boron, or at least 100 ppm boron to the lubricant composition. In certain embodiments, the lubricant composition may be free of a boron-containing dispersant, i.e. delivers no more than 10 ppm boron to the final formulation.

The dispersant may be prepared/obtained/obtainable from reaction of succinic anhydride by an “ene” or “thermal” reaction, by what may be referred to as a “direct alkylation process”. The “ene” reaction mechanism and general reaction conditions are summarized in “Maleic Anhydride”, pages, 147-149, Edited by B. C. Trivedi and B. C. Culbertson and Published by Plenum Press in 1982. The dispersant prepared by a process that includes an “ene” reaction may be a polyisobutylene succinimide having a carbocyclic ring present on less than 50 mole %, or 0 to less than 30 mole %, or 0 to less than 20 mole %, or 0 mole % of the dispersant molecules. The “ene” reaction may have a reaction temperature of 180 °C. to less than 300 °C., or 200 °C. to 250 °C., or 200 °C. to 220 °C.

The dispersant may also be obtained/obtainable from a chlorine-assisted process, often involving Diels-Alder chemistry, leading to formation of carbocyclic linkages. The process is known to a person skilled in the art. The chlorine-assisted process may produce a dispersant that is a polyisobutylene succinimide having a carbocyclic ring present on 50 mole % or more, or 60 to 100 mole % of the dispersant molecules. Both the thermal and chlorine-assisted processes are described in greater detail in U.S. Pat. No. 7,615,521, columns 4-5 and preparative examples A and B.

The dispersant may have a carbonyl to nitrogen ratio (CO:N ratio) of 5:1 to 1:10, 2:1 to 1:10, or 2:1 to 1:5, or 2:1 to 1:2. In certain embodiments the dispersant may have a CO:N ratio of 2:1 to 1:10, or 2:1 to 1:5,or 2:1 to 1:2,or 1:1.4 to 1:0.6.

In certain embodiments, the dispersant may be a succinimide dispersant may comprise a polyisobutylene succinimide, wherein the polyisobutylene from which polyisobutylene succinimide is derived has a number average molecular weight of 350 to 5000, or 750 to 2500. The dispersant may be present at 0 wt % to 20 wt %, 0.1 wt % to 15 wt %, or 0.5 wt % to 9 wt %, or 1 wt % to 8.5 wt % or 1.5 to 5 wt % of the lubricant composition.

In certain embodiments, an engine oil lubricant composition comprising the succinimides, succinamides, or succinate esters of the present technology may be a lubricant composition further comprising a molybdenum compound. The molybdenum compound may be an antiwear agent or an antioxidant. The molybdenum compound may be chosen from molybdenum dialkyldithiophosphates, molybdenum dithiocarbamates, amine salts of molybdenum compounds, and mixtures thereof. The molybdenum compound may provide the lubricant composition with 0 to 1000 ppm, or 5 to 1000 ppm, or 10 to 750 ppm 5 ppm to 300 ppm, or 20 ppm to 250 ppm of molybdenum.

In certain embodiments, an engine oil lubricant composition comprising succinimides, succinamides, and succinate esters of the present technology may further comprise an antioxidant. Antioxidants include sulfurized olefins, diarylamines, alkyated diarylamines, hindered phenols, molybdenum compounds (such as molybdenum dithiocarbamates), hydroxyl thioethers, or mixtures thereof. In certain embodiments the lubricant composition includes an antioxidant, or mixtures thereof. The antioxidant may be present at 0 wt % to 15 wt %, or 0.1 wt % to 10 wt %, or 0.5 wt % to 5 wt %, or 0.5 wt % to 3 wt %, or 0.3 wt % to 1.5 wt % of the lubricant composition.

In certain embodiments, the dispersant may be a mixture comprising: (i) a non-borated dispersant that comprises a polyisobutylene succinimide, wherein the polyisobutylene used to prepare the non-borated dispersant has a number average molecular weight of 550 to 1150, or 750 to 1150, or 900 to 1000 (often commercially available with a number average molecular weight of about 950); and (ii) a borated dispersant that comprises a polyisobutylene succinimide, wherein the polyisobutylene used to prepare the non-borated dispersant has a number average molecular weight of 550 to 1150, or 750 to 1150, or 900 to 1000 (often commercially available with a number average molecular weight of about 950).

In certain embodiments, the dispersant may be a mixture comprising: (a) a non-borated dispersant that comprises a polyisobutylene succinimide, wherein the polyisobutylene used to prepare the non-borated dispersant has a number average molecular weight of 550 to 1150, or 750 to 1150, or 900 to 1000 (often commercially available with a number average molecular weight of about 950); and (b) a borated dispersant may also be a product prepared by heating together: (i) a dispersant substrate; (ii) 2,5-dimercapto-1,3,4-thiadiazole or a hydrocarbylsubstituted 2,5-di-mercapto-1,3,4-thiadiazole, or oligomers thereof; (iii) a borating agent; and (iv) optionally a dicarboxylic acid of an aromatic compound chosen from 1,3 diacids and 1,4 diacids; or (v) optionally a phosphorus acid compound, said heating being sufficient to provide a product of (i), (ii), (iii) and optionally (iv) or (v), which is soluble in an oil of lubricating viscosity.

### Antioxidants

In certain embodiments, an engine oil lubricant composition comprising the succinimides, succinamides, and succinate esters of the present technology and further comprises a phenolic or an aminic antioxidant or mixtures thereof, and wherein the antioxidant is present at 0.1 wt % to 3 wt %, or 0.5 wt % to 2.75 wt %, or 1 wt % to 2.5 wt %. In another embodiment the antioxidant when present, may be present at 0.2 wt % to 1.2 wt %, or 0.3 wt % to 1.0 wt %, or 0.4 wt % to 0.9 wt % or 0.5 wt % to 0.8 wt %, of the lubricant.

The diarylamine or alkylated diarylamine may be a phenyl-a-naphthylamine (PANA), an alkylated diphenylamine, or an alkylated phenylnapthylamine, or mixtures thereof. The alkylated diphenylamine may include di-nonylated diphenylamine, nonyl diphenylamine, octyl diphenylamine, di-octylated diphenylamine, di-decylated diphenylamine, decyl diphenylamine and mixtures thereof. In certain embodiments the diphenylamine may include nonyl diphenylamine, di-nonyl diphenylamine, octyl diphenylamine, di-octyl diphenylamine, or mixtures thereof. In certain embodiments the alkylated diphenylamine may include nonyl diphenylamine, or di-nonyl diphenylamine. The alkylated diarylamine may include octyl, di-octyl, nonyl, di-nonyl, decyl or di-decyl phenylnapthylamines.

The hindered phenol antioxidant often contains a secondary butyl and/or a tertiary butyl group as a sterically hindering group. The phenol group may be further substituted with a hydrocarbyl group (typically linear or branched alkyl) and/or a bridging group linking to a second aromatic group. Examples of suitable hindered phenol antioxidants include 2,6-di-tert-butylphenol, 4-methyl-2,6-di-tert-butylphenol, 4-ethyl-2,6-di-tert-butylphenol, 4-propyl-2,6-di-tert-butylphenol or 4-butyl-2,6-di-tert-butylphenol, or 4-dodecyl-2,6-di-tert-butylphenol. In certain embodiments the hindered phenol antioxidant may be an ester and may include, e.g., Irganox™ L-135 from Ciba. A more detailed description of suitable ester-containing hindered phenol antioxidant chemistry is found in U.S. Pat. No. 6,559,105. If present, the phenolic antioxidant may be present at 0.1 wt % to 2 wt %, or 0.2 wt % to 1.5 wt % or 0.1 wt % to 1 wt %, or 0.4 wt % to 1.0 wt %, of the lubricant composition.

Examples of molybdenum dithiocarbamates, which may be used as an antioxidant, include commercial materials sold under the trade names such as Molyvan 822@, Molyvan® A and Molyvan® 855 from R. T. Vanderbilt Co., Ltd., and Adeka Saknra-Lube™ S-100, S-165, S-600 and 525, or mixtures thereof.

### Viscosity Modifier

In certain embodiments, an engine oil lubricant composition comprising the succinimides, succinamides, and succinate esters of the present technology further includes a viscosity modifier. The viscosity modifier is known in the art and may include hydrogenated styrene-butadiene rubbers, ethylene-propylene copolymers, ethylene copolymers with propylene and higher olefins, polymethacrylates, polyacrylates, hydrogenated styrene-isoprene polymers, hydrogenated diene polymers, polyalkyl styrenes, polyolefins, esters of maleic anhydride-olefin copolymers (such as those described in International Application WO 2010/014655), esters of maleic anhydride-styrene copolymers, or mixtures thereof. The viscosity modifier may include a block copolymer comprising (i) a vinyl aromatic monomer block and (ii), a conjugated diene olefin monomer block (such as a hydrogenated styrene-butadiene copolymer or a hydrogenated styrene-isoprene copolymer), a polymethacrylate, an ethylene-alpha olefin copolymer, a hydrogenated star polymer comprising conjugated diene monomers such as butadiene or isoprene, or a star polymer of polymethacrylate, or mixtures thereof.

### Dispersant Viscosity Modifier

In certain embodiments, the viscosity modifier may be a dispersant viscosity modifier. The dispersant viscosity modifier may include functionalized polyolefins, for example, ethylene-propylene copolymers that have been functionalized with an acylating agent such as maleic anhydride and an amine.

In certain embodiments, the dispersant viscosity modifier comprises an olefin copolymer further functionalized with a dispersant amine group. Typically, the olefin copolymer is an ethylene-propylene copolymer. The olefin copolymer has a number average molecular weight of 5000 to 20,000, or 6000 to 18,000, or 7000 to 15,000. The olefin copolymer may have a shear stability index of 0 to 20, or 0 to 10, or 0 to 5 as measured by the Orbahn shear test (ASTM D6278) as described above.

The formation of a dispersant viscosity modifier is well known in the art. The dispersant viscosity modifier may include for instance those described in U.S. Pat. No. 7,790,661 column 2, line 48 to column 10, line 38.

In certain embodiments, the dispersant viscosity modifier may be prepared by grafting of an olefinic carboxylic acid acylating agent onto a polymer of 15 to 80 mole percent of ethylene, from 20 to 85 mole percent of C₃₋₁₀ a-monoolefin, and from 0 to 15 mole percent of non-conjugated diene or triene, said polymer having an average molecular weight ranging from 5000 to 20,000, and further reacting said grafted polymer with an amine (typically an aromatic amine).

The dispersant viscosity modifier may include functionalized polyolefins, for example, ethylene-propylene copolymers that have been functionalized with an acylating agent such as maleic anhydride and an amine; polymethacrylates functionalized with an amine, or styrene-maleic anhydride copolymers reacted with an amine. Suitable amines may be aliphatic or aromatic amines and polyamines. Examples of suitable aromatic amines include nitroaniline, aminodiphenylamine (ADPA), hydrocarbylene coupled polyaromatic amines, and mixtures thereof. More detailed description of dispersant viscosity modifiers are disclosed in International Publication WO2006/015130 or U.S. Pat. Nos. 4,863,623; 6,107,257; 6,107,258; 6,117,825; and 7,790,661.

In certain embodiments, the dispersant viscosity modifier may include those described in U.S. Pat. No. 4,863,623 (see column 2, line 15 to column 3, line 52) or in International Publication WO2006/015130 (see page 2, paragraph [0008] and preparative examples are described paragraphs [0065] to [0073]). In certain embodiments the dispersant viscosity modifier may include those described in U.S. Pat. No. 7,790,661 column 2, line 48 to column 10, line 38.

In certain embodiments, an engine oil lubricant composition comprising the succinimides, succinamides, and succinate esters of the present technology further comprises a dispersant viscosity modifier. The dispersant viscosity modifier may be present at 0 wt % to 5 wt %, or 0 wt % to 4 wt %, or 0.05 wt % to 2 wt %, or 0.2 wt % to 1.2 wt % of the lubricant composition.

### Friction Modifier

In certain embodiments, an engine oil lubricant composition comprising in addition to the succinimides, succinamides and succinate of the present technology further includes a friction modifier. In certain embodiments the friction modifier may be chosen from long chain fatty acid derivatives of amines, long chain fatty esters, or derivatives of long chain fatty epoxides; fatty imidazolines; amine salts of alkylphosphoric acids; fatty alkyl tartrates; fatty alkyl tartrimides; fatty alkyl tartramides; fatty malic esters and imides, fatty (poly)glycolates; and fatty glycolamides. The friction modifier may be present at 0 wt % to 6 wt %, or 0.01 wt % to 4 wt %, or 0.05 wt % to 2 wt %, or 0.1 wt % to 2 wt % of the lubricant composition. As used herein the term “fatty alkyl” or “fatty” in relation to friction modifiers means a carbon chain having 10 to 22 carbon atoms, typically a straight carbon chain.

Examples of suitable friction modifiers include long chain fatty acid derivatives of amines, fatty esters, or fatty epoxides; fatty imidazolines such as condensation products of carboxylic acids and polyalkylene-polyamines; amine salts of alkylphosphoric acids; fatty alkyl tartrates; fatty alkyl tartrimides; fatty alkyl tartramides; fatty phosphonates; fatty phosphites; borated phospholipids, borated fatty epoxides; glycerol esters such as glycerol mono-oleate; borated glycerol esters; fatty amines; alkoxylated fatty amines; borated alkoxylated fatty amines; hydroxyl and polyhydroxy fatty amines including tertiary hydroxy fatty amines; hydroxy alkyl amides; metal salts of fatty acids; metal salts of alkyl salicylates; fatty oxazolines; fatty ethoxylated alcohols; condensation products of carboxylic acids and polyalkylene polyamines; or reaction products from fatty carboxylic acids with guanidine, aminoguanidine, urea, or thiourea and salts thereof.

Friction modifiers may also encompass materials such as sulfurized fatty compounds and olefins, molybdenum dialkyldithiophosphates, molybdenum dithiocarbamates, sunflower oil or soybean oil monoester of a polyol and an aliphatic car boxy lie acid.

In certain embodiments, the friction modifier may be a long chain fatty acid ester. In another embodiment the long chain fatty acid ester may be a mono-ester and in another embodiment the long chain fatty acid ester may be a triglyceride.

### Antiwear Agents

In certain embodiments, a lubricant composition comprising the succinimides, succinamides and/or succinate of the present technology may further include at least one antiwear agent. Examples of suitable antiwear agents include titanium compounds, tartaric acid derivatives such as tartrate esters, amides or tartrimides, malic acid derivatives, citric acid derivatives, glycolic acid derivatives, oil soluble amine salts of phosphorus compounds, sulfurized olefins, metal dihydrocarbyldithiophosphates (such as zinc dialkyldithiophosphates), phosphites (such as dibutyl phosphite), phosphonates, thiocarbamate-containing compounds, such as thiocarbamate esters, thiocarbamate amides, thiocarbamic ethers, alkylene-coupled thiocarbamates, and bis(S-alkyldithiocarbamyl) disulfides.

The antiwear agent may in certain embodiments include a tartrate or tartrimide as disclosed in International Publication WO 2006/044411 or Canadian Patent CA 1 183 125. The tartrate or tartrimide may contain alkyl-ester groups, where the sum of carbon atoms on the alkyl groups is at least 8. The antiwear agent may in certain embodiments include a citrate as is disclosed in US Patent Application 20050198894.

Another class of additives includes oil-soluble titanium compounds as disclosed in U.S. Pat. No. 7,727,943 and US2006/0014651. The oil-soluble titanium compounds may function as antiwear agents, friction modifiers, antioxidants, deposit control additives, or more than one of these functions. In certain embodiments the oil soluble titanium compound is a titanium (IV) alkoxide. The titanium alkoxide is formed from a monohydric alcohol, a polyol or mixtures thereof. The monohydric alkoxides may have 2 to 16, or 3 to 10 carbon atoms. In certain embodiments, the titanium alkoxide is titanium (IV) isopropoxide. In certain embodiments, the titanium alkoxide is titanium (IV) 2-ethylhexoxide. In certain embodiments, the titanium compound comprises the alkoxide of a vicinal 1,2-diol or polyol. In certain embodiments, the 1,2-vicinal dial comprises a fatty acid mono-ester of glycerol, often the fatty acid is oleic acid.

In certain embodiments, the oil soluble titanium compound is a titanium carboxylate. In certain embodiments, the titanium (IV) carboxylate is titanium neodecanoate.

In certain embodiments, the antiwear agent may be a phosphorus-containing antiwear agent, which may be a zinc dialkyldithiophosphate, phosphite, phosphate, phosphonate, and ammonium phosphate salts, or mixtures thereof.

In certain embodiments, the lubricant composition may further comprise a phosphorus-containing antiwear agent, typically zinc dialkyldithiophosphate. Zinc dialkyldithiophosphates are known in the art. Examples of zinc dithiophosphates include zinc isopropyl methylamyl dithiophosphate, zinc isopropyl isooctyl dithiophosphate, zinc di(cyclohexyl) dithiophosphate, zinc isobutyl 2-ethylhexyl dithiophosphate, zinc isopropyl 2-ethylhexyl dithiophosphate, zinc isobutyl isoamyl dithiophosphate, zinc isopropyl n-butyl dithiophosphate, and combinations thereof. Zinc dialkyldithiophosphate may be present in amount to provide 0.01 wt % to 0.1 wt % phosphorus to the lubricating composition, or to provide 0.015 wt % to 0.075 wt % phosphorus, or 0.02 wt % to 0.05 wt % phosphorus to the lubricating composition.

In certain embodiments, the lubricant composition further comprises one or more zinc dialkyldithiophosphate such that the amine (thio)phosphate additive provides at least 50% of the total phosphorus present in the lubricating composition, or at least 70% of the total phosphorus, or at least 90% of the total phosphorus in the lubricating composition. In certain embodiments, the lubricant composition is free or substantially free of a zinc dialkyldithiophosphate.

The antiwear agent may be present at 0 wt % to 3 wt %, or 0.1 wt % to 1.5 wt %, or 0.5 wt % to 0.9 wt % of the lubricant composition.

In certain embodiments, the lubricant composition comprising in addition to the succinimides, succinamides and succinate of the present technology further comprises 0.01 to 5 wt % or 0.1 to 2 wt % of an ashless antiwear agent represented by Formula:

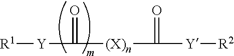

wherein Y and Y′ are independently —O—, >NH, >NR³, or an imide group formed by taking together both Y and Y′ groups and forming a R¹—N<group between two >C═O groups; X is independently —Z—O—Z′—, >CH₂, >CHR⁴, >CR⁴R⁵, >C(OH)(CO₂R²), >C(CO₂R²)₂, or >CHOR⁶; Z and Z′ are independently >CH₂, >CHR⁴, >CR⁴R⁵, >C(OH)(CO₂R²), or >CHOR⁶; n is 0 to 10, with the proviso that when n=1, X is not >CH₂, and when n=2, both X's are not >CH₂; m is 0 or 1; R¹ is independently hydrogen or a hydrocarbyl group, typically containing 1 to 150 carbon atoms, with the proviso that when R¹ is hydrogen, m is 0, and n is more than or equal to 1; R² is a hydrocarbyl group, typically containing 1 to 150 carbon atoms; R³, R⁴ and R⁵ are independently hydrocarbyl groups; and R⁶ is hydrogen or a hydrocarbyl group, typically containing 1 to 150 carbon atoms.

In certain embodiments, an engine oil lubricant composition comprising the succinimides, succinamides and succinate of the present technology further comprises 0.01 to 5 wt % or 0.1 to 2 wt % of an ashless antiwear agent that may be a compound obtained/obtainable by a process comprising reacting a glycolic acid, a 2-halo-acetic acid, or a lactic acid, or an alkali or alkaline metal salt thereof, (typically glycolic acid or a 2-halo-acetic acid) with at least one member selected from the group consisting of an amine, an alcohol, and an amino alcohol. For example, the compound may be represented by formula:

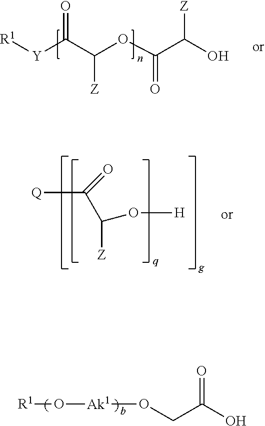

wherein Y is independently oxygen or >NH or >NR¹; R¹ is independently a hydrocarbyl group, typically containing 4 to 30, or 6 to 20, or 8 to 18 carbon atoms; Z is hydrogen or methyl; Q is the residue of a dial, trio! or higher polyol, a diamine, triamine, or higher polyamine, or an aminoalcohol (typically Q is a dial, diamine or aminoalcohol) g is 2 to 6, or 2 to 3, or 2; q is 1 to 4, or 1 to 3 or 1 to 2; n is 0 to 10, 0 to 6, 0 to 5, 1 to 4, or 1 to 3; and Ak:1 is an alkylene group containing 1 to 5, or 2 to 4 or 2 to 3 (typically ethylene) carbon atoms; and b is 1 to 10, or 2 to 8, or 4 to 6, or 4.

The compound is known and is described in International publication WO 2011/022317, and also in granted U.S. Pat. Nos. 8,404,625, 8,530,395, and 8,557,755.

In certain embodiments, the lubricant composition disclosed herein contains no zinc dialkyldithiophosphate.

In certain embodiments, the lubricant composition disclosed herein contains zinc dialkyldithiophosphate.

In certain embodiments, the antiwear agent is a phosphorus-containing compound. In certain embodiments, the phosphorous-containing compound may be a non-ionic phosphorus compound.

In certain embodiment, the phosphorus-containing compounds comprise two or more (possibly up to four) non-ionic phosphorus compounds. Typically, the non-ionic phosphorus compound may have an oxidation of +3 or +5. The different embodiments comprise phosphite ester, phosphate esters, or mixtures thereof.

In certain embodiments, the phosphorus-containing compound comprises a non-ionic phosphorus compound (a C₄₋₆ hydrocarbyl phosphite) and an amine salt of a phosphorus acid or ester.

The phosphorus-containing compound comprises a nonionic phosphorus compound that is a C₄₋₆ hydrocarbyl phosphite, or mixtures thereof. The C₄₋₆ hydrocarbyl phosphite includes those represented by the formula:

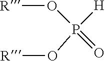

wherein each R′″ may be independently hydrogen or a hydrocarbyl group having 4 to 6 carbon atoms, typically 4 carbon atoms, with the proviso that at least one of the R′″ groups is hydrocarbyl. Typically, the C₄₋₆ hydrocarbyl phosphite comprises dibutyl phosphite.

The C₄₋₆ hydrocarbyl phosphite may deliver at least 175 ppm, or at least 200 ppm of the total amount of phosphorus delivered by the phosphorus-containing compounds.

The C₄₋₆ hydrocarbyl phosphite may deliver at least 45 wt %, or 50 wt % to 100 wt %, or 50 wt % to 90 wt % or 60 wt % to 80 wt % of the total amount of phosphorus from the phosphorus-containing compound.

The phosphorus-containing compounds may comprise a second phosphite whose formula is similar to that disclosed above, except R′″ may contain 2 to 40, 8 to 24 or 11 to 20 carbon atoms, with the proviso that the second phosphite is not a C₄₋₆ hydrocarbyl phosphite. Examples of suitable hydrocarbyl groups include propyl, dodecyl, butadecyl, hexadecyl, octadecyl, propenyl, dodecenyl, butadecenyl, hexadeencyl, or octadecenyl groups.

As used herein, the term “alk(en)yl” is intended to include moieties that have an alkyl and/or alkenyl group.

In certain embodiments, the phosphorus-containing compounds include a mixture of a C C₄₋₆ hydrocarbyl phosphite (typically dibutyl phosphite) and a C₁₂₋₈ alk(en)yl hydrogen phosphite and optionally phosphoric acid. In different embodiments the phosphoric acid is present or absent.

In certain embodiments, the phosphorus-containing compounds include a mixture of a C₄₋₆ hydrocarbyl phosphite (typically dibutyl phosphite) and a C₁₆₋₁₈ alk(en)yl hydrogen phosphite. The alk(en)yl hydrogen phosphite be may an alkyl hydrogen phosphite, and alkenyl hydrogen phosphite, or a mixture of alkenyl hydrogen phosphite and alkyl hydrogen phosphite. In certain embodiments the alk(en)yl hydrogen phosphite be may a mixture of alkenyl hydrogen phosphite and alkyl hydrogen phosphite and optionally phosphoric acid. The phosphoric acid may be present or absent.

In certain embodiments, the phosphorus-containing compounds include a mixture of a C₄₋₆ hydrocarbyl phosphite (typically dibutyl phosphite) and a C₁₁₋₁₄ alk(en)yl hydrogen phosphite. The alk(en)yl hydrogen phosphite may be an alkyl hydrogen phosphite, and alkenyl hydrogen phosphite, or a mixture of alkenyl hydrogen phosphite and alkyl hydrogen phosphite. In certain embodiments, the alk(en)yl hydrogen phosphite may be a mixture of alkenyl hydrogen phosphite and alkyl hydrogen phosphite and optionally phosphoric acid.

In certain embodiments, the phosphorus-containing compounds include a mixture of a C₄₋₆ hydrocarbyl phosphite (typically dibutyl phosphite) and phosphoric acid.

In certain embodiments, the lubricant compositions comprises a package that comprises a phosphorus-containing compound and a non-ionic phosphorus compound that is a hydrocarbyl phosphite.

In certain embodiments, the lubricant composition further comprises a C₈₋₂₀ hydrocarbyl phosphite, or a C₁₂₋₁₈ hydrocarbyl phosphite, or C₁₆₋₁₈ hydrocarbyl phosphite.

In certain embodiments, the amine salt of a phosphorus acid. The amine salt of a phosphorus acid may be derived from an amine salt of a phosphate. The amine salt of the phosphorus acid may be represented by the formula:

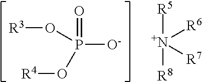

wherein R³ and R⁴ may be independently hydrogen or hydrocarbon typically containing 4 to 40, or 6 to 30, or 6 to 18, or 8 to 18 carbon atoms, with the proviso that at least one is a hydrocarbon group; and R⁵, R⁶, R⁷ and R⁸ may be independently hydrogen or a hydrocarbyl group, with the proviso that at least one is a hydrocarbyl group. The hydrocarbon groups of R³ and/or R⁴ may be linear, branched, or cyclic. Examples of a hydrocarbon group for R³ and/or R⁴ include straight-chain or branched alkyl groups include methyl, ethyl, propyl, butyl, pentyl, hexyl, heptyl, octyl, nonyl, decyl, undecyl, dodecyl, tridecyl, tetradecyl, pentadecyl, hexadecyl, heptadecyl and octadecyl. Examples of a cyclic hydrocarbon group for R³ and/or R⁴ include cyclopentyl, cyclohexyl, cycloheptyl, methylcyclopentyl, dimethylcyclopentyl, methylcyclopentyl, dimethylcyclopentyl, methylethylcyclopentyl, diethylcyclopentyl, methylcyclohexyl, dimethylcyclohexyl, methylethylcyclohexyl, diethylcyclohexyl, methylcycloheptyl, dimethylcycloheptyl, methylethylcycloheptyl, and diethylcycloheptyl.

In certain embodiments, the phosphate may be an amine salt of a mixture of monoalkyl and dialkyl phosphoric acid esters. The monoalkyl and dialkyl groups may be linear or branched.

The amine salt of a phosphorus acid may be derived from an amine such as a primary amine, a secondary amine, a tertiary amine, or mixtures thereof. The amine may be aliphatic, or cyclic, aromatic or non-aromatic, typically aliphatic. In certain embodiments, the amine includes an aliphatic amine such as a tertiary-aliphatic primary amine.

Examples of suitable primary amines include ethylamine, propylamine, butylamine, 2-ethylhexylamine, bis-(2-ethylhexyl)amine, octylamine, and dodecyl-amine, as well as such fatty amines as n-octylamine, n-decylamine, n-dodecylamine, n-tetradecylamine, n-hexadecylamine, n-octadecylamine and oleyamine. Other useful fatty amines include commercially available fatty amines such as “Armeen®” amines (products available from Akzo Chemicals, Chicago, Ill.), such as Armeen C, Armeen O, Armeen OL, Armeen T, Armeen HT, Armeen S and Armeen SD, wherein the letter designation relates to the fatty group, such as coco, oleyl, tallow, or stearyl groups.

Examples of suitable secondary amines include dimethylamine, diethylamine, dipropylamine, dibutylamine, diamylamine, dihexylamine, diheptylamine, methylethylamine, ethylbutylamine, N-methyl-1-amino-cyclohexane, Armeen® 2C and ethylamylamine. The secondary amines may be cyclic amines such as piperidine, piperazine and morpholine.

Examples of tertiary amines include tri-n-butylamine, tri-n-octylamine, tri-decylamine, tri-laurylamine, tri-hexadecylamine, and dimethyloleylamine (Armeen® DMOD).

In certain embodiments, the amines are in the form of a mixture. Examples of suitable mixtures of amines include (i) a tertiary alkyl primary amine with 11 to 14 carbon atoms, (ii) a tertiary alkyl primary amine with 14 to 18 carbon atoms, or (iii) a tertiary alkyl primary amine with 18 to 22 carbon atoms. Other examples of tertiary alkyl primary amines include tert-butylamine, tert-hexylamine, tert-octylamine (such as 1,1-dimethylhexylamine), tert-decylamine (such as 1,1-dimethyloctylamine), tertdodecylamine, tert-tetradecylamine, tert-hexadecylamine, tert-octadecylamine, tert-tetracosanylamine, and tert-octacosanylamine.

In certain embodiments, a useful mixture of amines is “Primene® 81R” or “Primene® JMT.” Primene® 81R and Primene® JMT (both produced and sold by Rohm & Haas) are mixtures of Cll to C14 tertiary alkyl primary amines and C18 to C22 tertiary alkyl primary amines respectively.

The amine salt of a phosphorus acid may be prepared as is described in U.S. Pat. No. 6,468,946. Column 10, lines 15 to 63 describes phosphoric acid esters formed by reaction of phosphorus compounds, followed by reaction with an amine to form an amine salt of a phosphate hydrocarbon ester. Column 10, line 64, to column 12, line 23, describes preparative examples of reactions between phosphorus pentoxide with an alcohol (having 4 to 13 carbon atoms), followed by a reaction with an amine (typically Primene®81-R) to form an amine salt of a phosphate hydrocarbon ester.

### Sulfur-Containing Extreme Pressure Agent

The sulfur-containing extreme pressure agent may be an olefin sulfide, or mixtures thereof. The olefin sulfide may include a polysulfide or a sulfurized olefin such as sulfurized isobutylene, or mixtures thereof.

In certain embodiments, the olefin sulfide includes a polysulfide.

In certain embodiments, the olefin sulfide includes sulfurized isobutylene.

In certain embodiments, the olefin sulfide includes a mixture of a sulfurized isobutylene and a polysulfide.

In certain embodiments, at least 50 wt % of the polysulfide molecules are a mixture of tri- or tetra-sulfides. In certain embodiments, at least 55 wt %, or at least 60 wt % of the polysulfide molecules are a mixture of tri- or tetra-sulfides.

The polysulfide includes a sulfurized organic polysulfide from oils, fatty acids or ester, olefins or polyolefins.

Oils which may be sulfurized include natural or synthetic oils such as mineral oils, lard oil, carboxylate esters derived from aliphatic alcohols and fatty acids or aliphatic carboxylic acids (e.g., myristyl oleate and oleyl oleate), and synthetic unsaturated esters or glycerides.

Fatty acids include those that contain 8 to 30, or 12 to 24 carbon atoms. Examples of fatty acids include oleic, linoleic, linolenic, and tall oil. sulfurized fatty acid esters prepared from mixed unsaturated fatty acid esters such as are obtained from animal fats and vegetable oils, including tall oil, linseed oil, soybean oil, rapeseed oil, and fish oil.

The polysulfide includes olefins derived from a wide range of alkenes. The alkenes typically have one or more double bonds. The olefins in certain embodiments contain 3 to 30 carbon atoms. In other embodiments, olefins contain 3 to 16, or 3 to 9 carbon atoms. In certain embodiments, the sulfurized olefin includes an olefin derived from propylene, isobutylene, pentene or mixtures thereof.

In certain embodiments, the polysulfide comprises a polyolefin derived from polymerizing by known techniques, an olefin as described above.

In certain embodiments, the polysulfide includes dibutyl tetrasulfide, sulfurized methyl ester of oleic acid, sulfurized alkylphenol, sulfurized dipentene, sulfurized dicyclopentadiene, sulfurized terpene, and sulfurized Diels-Alder adducts.

### Other Additives

The lubricant composition may also include a sulfur-containing corrosion inhibitor. The sulfur-containing corrosion inhibitor may be a thiadiazole compound, or mixtures thereof. The thiadiazole compound may include mono- or di-hydrocarbyl substituted 2,5-dimercapto-1,3,4-thiadiazole compounds. Examples of a thiadiazole include 2,5-dimercapto-1,3,4-thiadiazole, or oligomers thereof, a hydrocarbyl-substituted 2.5-dimercapto-1,3,4-thiadiazole, a hydrocarbylthio-substituted 2,5-dimercapto-1,3,4-thiadiazole, or oligomers thereof. The oligomers of hydrocarbyl-substituted 2,5-dimercapto-1,3,4-thiadiazole typically form by forming a sulfur-sulfur bond between 2,5-dimercapto-1,3,4-thiadiazole units to form oligomers of two or more of said thiadiazole units. These thiadiazole compounds may also be used in the post treatment of dispersants as mentioned below in the formation of a dimercaptothiadiazole derivative of a polyisobutylene succinimide.

Examples of a suitable thiadiazole compound include at least one of a dimercaptothiadiazole, 2,5-dimercapto-[1,3, 4]-thiadiazole, 3,5-dimercapto-[1,2,4]-thiadiazole, 3,4-dimercapto-[1,2,5]-thiadiazole, or 4-5-dimercapto-[1,2,3]-thiadiazole. Typically, readily available materials such as 2.5-dimercapto-1,3,4-thiadiazole or a hydrocarbyl-substituted 2,5-dimercapto-1,3,4-thiadiazole or a hydrocarbylthiosubstituted 2,5-dimercapto-1,3,4-thiadiazole are commonly utilized.

Foam inhibitors that may be useful in the compositions described herein include polysiloxanes, copolymers of ethyl acrylate and 2-ethylhexylacrylate and optionally vinyl acetate; demulsifiers including fluorinated polysiloxanes, trialkyl phosphates, polyethylene glycols, polyethylene oxides, polypropylene oxides and (ethylene oxide-propylene oxide) polymers.

Pour point depressants that may be useful in the compositions described herein include polyalphaolefins, esters of maleic anhydride-styrene copolymers, poly(meth)acrylates, polyacrylates or polyacrylamides.

Demulsifiers include trialkyl phosphates, and various polymers and copolymers of ethylene glycol, ethylene oxide, propylene oxide, or mixtures thereof.

Metal deactivators may be chosen from a derivative of benzotriazole (typically tolyltriazole), 1,2,4-triazole, benzimidazole, 2-alkyldithiobenzimidazole or 2-alkyldithiobenzothiazole, 1-amino-2-propanol, a derivative of dimercaptothiadiazole, octylamine octanoate, condensation products of dodecenyl succinic acid or anhydride and/or a fatty acid such as oleic acid with a polyamine. The metal deactivators may also be described as corrosion inhibitors.

Seal swell agents include sulpholene derivatives Exxon Necton-37™ (FN 1380) and Exxon Mineral Seal Oil™ (FN 3200).

### EXAMPLES

The subject matter disclosed herein may be better understood with reference to the following examples, which are set forth merely to further illustrate the subject matter disclosed herein. The illustrative examples should not be construed as limiting the subject matter in any manner.

The following examples were generally prepared according to the following representative preparation scheme for preparing succinimide or succinamide derivatives from a PIBSA product (either a radically-functionalized PIBSA Product, referred to as “RF”, or a conventional PIBSA product prepared from a conventional thermal process, referred to as “Thermal”): A round-bottom flask equipped with a Dean-Stark trap, addition funnel, temperature probe, and overhead stirrer was charged with the PIBSA and diluent oil. This mixture was heated to about 110-120 °C and the amine was added dropwise over 30 to 60 minutes. Following addition of the amine, the reaction mixture was heated to about 150-160 °C and held at 150-160 °C for about 4-6 hours while removing water of reaction. Once the removal of water of reaction was complete, the reaction mixture was cooled and poured into a jar. In some instances, the derived succinamide or succinimide is post treated with post treating agents such as boric acid, phosphorus acids, dimecaptothiadiazole, or an aromatic diacid.

The dispersants described in Table 1 were prepared according to the process described in the previous paragraph.

TABLE 1

| | PIBSA | PIB | | | | | | | % |
|---|---|---|---|---|---|---|---|---|---|
| Example | Type | Mn | Amine | % N | TBN | % B | % P | % S | Oil |
| INV D1 | RF | 1550^{A} | TETA^{D} | 1.08 | 16 | — | — | — | 29 |
| INV D2 | RF | 1550^{B} | TETA^{D} | 1.03 | 15.6 | — | — | — | 29 |
| COMP D1 | Thermal | 1550 | TETA^{D} | 0.98 | 12 | — | — | — | 29 |
| INV D3 | RF | 1000 | PHPA^{C} | 2.16 | 51.4 | 0.45 | — | — | 33 |
| COMP D2 | Thermal | 1000 | PHPA^{C} | 2.12 | 50.9 | 0.46 | — | — | 43 |
| INV D4 | RF | 1000 | PHPA^{C} | 2.22 | 49.9 | 0.45 | — | 0.37 | 33 |
| COMP D3 | Thermal | 1000 | PHPA^{C} | 2.23 | 48.3 | 0.44 | — | 0.32 | 43 |
| INV D5 | RF | 1000 | PHPA^{C} | 2.33 | 63 | 1.77 | — | — | 37 |
| COMP D4 | Thermal | 1000 | DBDA^{E} | 2.33 | 63.8 | 1.95 | — | — | 33 |
| COMP D5 | Thermal | 1000 | TETA^{D} | 2.14 | 49 | 2.02 | — | — | 40 |
| INV D6 | RF | 1000 | TETA^{D} | 1.95 | 47 | 1.80 | — | — | 33 |
| INV D7 | RF | 1000 | PHPA^{C} | 1.63 | 31.4 | — | — | — | 29 |
| INV D8 | RF | 1000 | PHPA^{C} | 2.10 | 49.7 | — | — | — | 33 |

^{A}60:40 mixture of 2300 Mn high vinylidene PIB and 1000 Mn high vinylidene PIB
^{B}85:15 mixture of 2300 Mn high vinylidene PIB and 550 Mn high vinylidene PIB
^{C}Polyethyleneamine bottoms
^{D}Tetraethylenetriamine
^{E}85:15 Mixture of polyethyleneamine bottoms and diethylenetriamine

Inventive examples IN4V D1 and IN4V D2 were evaluated in a commercial engine oil lubricant for piston deposit control in the EA888 piston cleanliness test and the and VW TDi3 piston deposit test. Both tests evaluate the ability of the additive package to minimize the amount of deposits on pistons during operation of the engine. The data shown in Table 2 indicates that the inventive examples prepared from a radical functionalized polyisobutylene succinic anhydride has equivalent piston deposit performance, at an equal actives treat rate to a comparative dispersant COMP D1 prepared from a polyisobutylene succinic anhydride made via the well-known thermal process in the absence of radical initiators.

TABLE 2

| | EO | EO | EO | EO |
|---|---|---|---|---|
| | Fluid 1 | Fluid 2 | Fluid 3 | Fluid 4 |
| INV D1 | 4.97 | 6.39 | | |
| INV D2 | | | 4.97 | |
| COMP D1 | | | | 4.97 |
| phosphorus-containing | 0.9 | 0.9 | 0.9 | 0.9 |
| antiwear agent | | | | |
| Overbased Detergent | 2.1 | 2.1 | 2.1 | 2.1 |
| Styrene/olefin Viscosity Modifier | 1.4 | 1.4 | 1.4 | 1.4 |
| Phenolic antioxidant | 1.0 | 1.0 | 1.0 | 1.0 |
| Aminic antioxidant | 1.0 | 1.0 | 1.0 | 1.0 |
| Other lubricant additives | 0.45 | 0.45 | 0.45 | 0.45 |
| Oil of Lubricating Viscosity | Balance | Balance | Balance | Balance |
| | to | to | to | to |
| | 100% | 100% | 100% | 100% |

VW TDi3 piston deposit test. (GAS_HOT_P_SC)

| | | | | |
|---|---|---|---|---|
| Groove and Land | 3.68 | 4.43 | 3.83 | 2.89 |
| Weighted Deposit | | | | |
| Average MERIT | | | | |
| Undercrown Weighted Deposit | 4.75 | 5.13 | 4.7 | 4.32 |
| Average MERIT | | | | |
| Undercrowns Weighted Deposit | 5.37 | 5.74 | 5.3 | 4.58 |
| Average MERIT | | | | |

VW TDi3 piston deposit test (PV1808)

| | | |
|---|---|---|
| Average Piston Deposit MERIT | 60 | 64 |

Inventive examples INV D5 and INV D6 were evaluated in a commercial grease for thermal resistance by the ASTM dropping point (D32256) tests The dropping point of a lubricating grease is an indication of the heat resistance of the grease and is the temperature at which it passes from a semi-solid to a liquid state under specific test conditions. It is dependent on the type of thickener used and the cohesiveness of the oil and thickener of a grease. The data shown in Table 2 indicates that the inventive examples INV D5 and INV D6 prepared from a radical functionalized polyisobutylene succinic anhydride has dropping point performance, at an equal actives treat rate, to the comparative dispersants COMP D1 prepared from a polyisobutylene succinic anhydride made via the well-known thermal process in the absence of radical initiators.

TABLE 3

| D2265 (Dropping point) | INV D5 | INV D6 | COMP D4 |
|---|---|---|---|
| Dropping Point | 259 °C | 259 °C | 262 259 °C |

Inventive examples INV D3 and INV D4 were evaluated in commercial transmission fluids to assess the shifting quality of an automatic transmission the fluids ability to prevent shudder in an automatic transmission and the friction characteristics of the fluid in a continuous variable transmission to prevent catastrophic damage to the transmission. The data shown in Table 4 indicates that the inventive examples prepared from an radical functionalized polyisobutylene succinic anhydride has equivalent frictional and antishudder performance in an automatic transmission when compared, at equal active treat rate to equal actives treat rate, to the comparative dispersants COMP D2 and COMP D3 prepared from a polyisobutylene succinic anhydride made via the well-known thermal process in the absence of radical initiators. The data shown in Table 5 indicates that the inventive examples INV D3 and INV D4 prepared from an radical functionalized polyisobutylene succinic anhydride has equivalent friction performance in a continuous variable transmission when compared to comparative dispersants COMP D2 and COMP D3 prepared from a polyisobutylene succinic anhydride made via the well-known thermal process in the absence of radical initiators.

Inventive examples INV D5 and INV D6 were evaluated in Commercial gear fluids to assessed in the L-60-1 oxidation test. The data shown in Table 7 indicates that the inventive examples prepared from an radical functionalized polyisobutylene succinic anhydride has equivalent oxidative stability in a gear oil fluid, at equal active treat rate, to comparative dispersants COMP D4 and COMP D5 prepared from a polyisobutylene succinic anhydride made via the well-known thermal process in the absence of radical initiators.

TABLE 4

| | ATF Fluid 1 | ATF fluid 2 |
|---|---|---|
| Commercial Fluid | Oil free % wt | Oil free % wt |
| INV D3 | 2.81 | — |
| INV D4 | 0.8 | — |
| COMP D2 | — | 2.39 |
| COMP D3 | — | 0.68 |
| Phosphorous Antiwear agent | 0.28 | 0.28 |
| Calcium Detergent | 0.24 | 0.24 |
| Friction Modifier | 1.22 | 1.22 |
| Thiadiazole | 0.08 | 0.08 |
| Antioxidant | 1.0 | 1.0 |
| Polymethacrylate Viscosity | 4.88 | 4.88 |
| Modifier | | |
| Other lubricant additives | 1.4 | 1.4 |
| Oil of Lubricating Viscosity | Balance to | Balance to |
| | 100% | 100% |

JASO SAE#2 Friction: M348: 2012.01

| | | |
|---|---|---|
| Minimum μₜ₂ (50-5000) | 0.113 | 0.105 |
| Minimum μₜ₂ (50-500) | 0.115 | 0.105 |

TABLE 5

| | |
|---|---|
| ATF Fluid 1 | ATF fluid 2 |

Mercon V 30K; 018.080.01 (BorgWarner SD-1777)

| | | | | RATIO | | | | | RATIO | |
|---|---|---|---|---|---|---|---|---|---|---|
| | MU- | MU- | MU- | E/M | LOCK | MU- | MU- | MU- | E/M | LOCK |
| CYCLES cyc/hr | MIPOINT | LOWSPEED | STATIC | (S1/D) | UP sec | MIPOINT | LOWSPEED | STATIC | (S1/D) | UP sec |
| 25 | 0.137 | 0.131 | 0.119 | 0.957 | 0.87 | 0.135 | 0.126 | 0.11 | 0.934 | 0.91 |
| 100 | 0.138 | 0.124 | 0.109 | 0.895 | 0.88 | 0.134 | 0.113 | 0.098 | 0.84 | 0.92 |
| 200 | 0.136 | 0.116 | 0.102 | 0.848 | 0.9 | 0.133 | 0.109 | 0.099 | 0.817 | 0.92 |
| 400 | 0.135 | 0.116 | 0.101 | 0.856 | 0.91 | 0.134 | 0.109 | 0.098 | 0.813 | 0.92 |
| 600 | 0.136 | 0.115 | 0.102 | 0.85 | 0.9 | 0.136 | 0.112 | 0.099 | 0.828 | 0.91 |
| 800 | 0.136 | 0.114 | 0.103 | 0.839 | 0.9 | 0.137 | 0.11 | 0.099 | 0.801 | 0.91 |
| 1000 | 0.138 | 0.115 | 0.103 | 0.837 | 0.89 | 0.138 | 0.112 | 0.101 | 0.81 | 0.9 |
| 2000 | 0.145 | 0.121 | 0.107 | 0.836 | 0.85 | 0.139 | 0.118 | 0.102 | 0.846 | 0.9 |
| 4000 | 0.141 | 0.126 | 0.11 | 0.894 | 0.89 | 0.138 | 0.12 | 0.105 | 0.873 | 0.89 |
| 6000 | 0.14 | 0.126 | 0.112 | 0.903 | 0.88 | 0.138 | 0.122 | 0.105 | 0.881 | 0.89 |
| 8000 | 0.141 | 0.128 | 0.113 | 0.906 | 0.88 | 0.138 | 0.123 | 0.107 | 0.887 | 0.89 |
| 10000 | 0.141 | 0.127 | 0.113 | 0.903 | 0.87 | 0.139 | 0.124 | 0.108 | 0.887 | 0.88 |
| 12000 | 0.142 | 0.127 | 0.113 | 0.899 | 0.87 | 0.139 | 0.124 | 0.109 | 0.893 | 0.9 |
| 14000 | 0.141 | 0.127 | 0.112 | 0.901 | 0.87 | 0.137 | 0.124 | 0.11 | 0.9 | 0.9 |
| 16000 | 0.141 | 0.127 | 0.112 | 0.898 | 0.88 | 0.137 | 0.124 | 0.11 | 0.907 | 0.9 |
| 18000 | 0.138 | 0.125 | 0 | 0.905 | 0.89 | 0.135 | 0.123 | 0.109 | 0.911 | 0.91 |
| 20000 | 0.139 | 0.126 | 0 | 0.904 | 0.89 | 0.134 | 0.123 | 0.11 | 0.917 | 0.92 |
| 22000 | 0.137 | 0.126 | 0.112 | 0.916 | 0.9 | 0.133 | 0.121 | 0.109 | 0.91 | 0.93 |
| 24000 | 0.136 | 0.124 | 0.112 | 0.909 | 0.9 | 0.132 | 0.121 | 0.109 | 0.912 | 0.93 |
| 26000 | 0.136 | 0.125 | 0.112 | 0.918 | 0.9 | 0.132 | 0.12 | 0.108 | 0.914 | 0.93 |
| 28000 | 0.133 | 0.122 | 0.111 | 0.919 | 0.92 | 0.131 | 0.119 | 0.109 | 0.908 | 0.94 |
| 30000 | 0.133 | 0.122 | 0.111 | 0.924 | 0.92 | 0.131 | 0.119 | 0.108 | 0.903 | 0.94 |

TABLE 6

| | CVT Fluid 1 | CVT Fluid 2 |
|---|---|---|
| | Oil free % wt | Oil free % wt |
| INV D1 | 2.68 | |
| INV D4 | 1.0 | |
| COMP D2 | | 2.28 |
| COMP D3 | | 0.85 |
| Phosphorous Antiwear agent | 0.42 | 0.42 |
| Calcium Detergent | 0.41 | 0.41 |
| Friction Modifier | 1.98 | 1.98 |
| Thiadiazole | 0.1 | 0.1 |
| Antioxidant | 0.8 | 0.8 |
| Polymethacrylate Viscosity Modifier | 3.91 | 3.91 |
| Other lubricant additives | 0.6 | 0.6 |
| Oil of Lubricating Viscosity | Balance to | Balance to |
| | 100% | 100% |

LFW-1 Metal-Metal Friction M358_05_HI

| | | |
|---|---|---|
| MU 1.0M/SEC | 0.134 | 0.136 |
| MU 0.5M/SEC | 0.14 | 0.14 |
| MU 0.25M/SEC | 0.142 | 0.142 |
| MU 0.125M/SEC | 0.144 | 0.143 |
| MU 0.075M/SEC | 0.145 | 0.143 |
| MU 0.025M/SEC | 0.143 | 0.139 |

LVFA JASO anti-shudder durability M349: 2012.01 Dynax D0600

| | | |
|---|---|---|
| Hours to negative slope | 240 | 240 |

TABLE 7

| | Gear | Gear | Gear | Gear |
|---|---|---|---|---|
| | Fluid 1 | Fluid 2 | Fluid 3 | Fluid 4 |
| | Oil | Oil | Oil | Oil |
| | free | free | free | free |
| | % wt | % wt | % wt | % wt |
| INV D5 | | | 1.25 | |
| INV D6 | | | | 1.25 |
| COMP D4 | 1.25 | | | |
| COMP D5 | | 1.25 | | |
| Thiadiazole | 0.17 | 0.17 | 0.17 | 0.17 |
| Olefin sulfide | 4.57 | 4.57 | 4.57 | 4.57 |
| Dithiophosphate extreme | 1.66 | 1.66 | 1.66 | 1.66 |
| pressure agent | | | | |
| Friction Modifier | 0.13 | 0.13 | 0.13 | 0.13 |
| Other lubricant additives | 0.07 | 0.07 | 0.07 | 0.07 |
| Oil of Lubricating Viscosity | Balance | Balance | Balance | Balance |
| | to 100% | to 100% | to 100% | to 100% |

L-60 (ASTM D5704) test results

| | | | | |
|---|---|---|---|---|
| Viscosity Increase (5) | 18 | 16 | 18 | 16 |
| Pentane insoluble (wt %) | 0.4 | 0.3 | 0.4 | 0.4 |
| Toluene insoluble (wt %) | 0.4 | 0.4 | 0.4 | 0.5 |
| Average Carbon/Varnish (merits | 7.5 | 8.3 | 7.1 | 8.4 |
| Average Sludge (merits) | 9.5 | 9.5 | 9.5 | 9.6 |

Except in the Examples, or where otherwise explicitly indicated or required by context, all numerical quantities in this description specifying amounts of materials, reaction conditions, molecular weights, number of carbon atoms, and the like, are to be understood as modified by the word “about”. As used herein, the term “about” means that a value of a given quantity is within ±2000 of the stated value. In other embodiments, the value is within ±±1500 of the stated value. In other embodiments, the value is within ±100% of the stated value. In other embodiments, the value is within ±500 of the stated value. In other embodiments, the value is within ±2.5% of the stated value. In other embodiments, the value is within ±10% of the stated value. In other embodiments, the value is within a range of the explicitly-described value which would be understood by those of ordinary skill, based on the disclosures provided herein, to perform substantially similarly to compositions including the literal amounts described herein.

It is to be understood that the upper and lower amount, range, and ratio limits set forth herein may be independently combined, and that any amount within a disclosed range is contemplated to provide a minimum or maximum of a narrower range in alternative embodiments (with the proviso, of course, that the minimum amount of a range must be lower than the maximum amount of the same range). Similarly, the ranges and amounts for each element of the subject matter disclosed herein may be used together with ranges or amounts for any of the other elements.

While certain representative embodiments and details have been shown for the purpose of illustrating the subject matter disclosed herein, it will be apparent to those skilled in this art that various changes and modifications may be made therein without departing from the scope of the subject matter. In this regard, the scope of the invention is to be limited only by the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A process for producing a reaction product, the process comprising: reacting a radically-functionalized PIBSA product with an amine or an alcohol to form the reaction product; wherein the reaction product comprises at least one of a succinimide, a succinamide, or a succinate ester.

22. The process of claim 21, wherein the amine is a polyamine.

23. The process of claim 22, wherein the polyamine contains at least one >N—H group per molecule.

24. The process of claim 21, wherein the polyamine is an aliphatic polyamine.

25. The process of claim 24, wherein the aliphatic polyamine is at least one of an ethylenepolyamine, a propylenepolyamine, or a butylenepolyamine.

26. The process of claim 25, wherein the ethylenepolyamine comprises at least one of ethylenediamine, diethylenetriamine, triethylenetetramine, tetraethylenepentamine, pentaethylenehexamine, or polyamine still bottoms.

27. The process of claim 21, wherein the alcohol is a polyhydric aliphatic alcohol.

28. The process of claim 27, wherein the polyhydric aliphatic alcohol is chosen from glycerol, pentaerythritol, or sorbitol.

29. The process of claim 21, wherein the reaction product comprises a succinate ester, and wherein the succinate ester is further reacted with an amine.

30. (canceled)

31. (canceled)

32. (canceled)

33. (canceled)

34. The process of claim 21, wherein the succinimide, succinamide or succinate ester is post-treated with a post-treating agent to produce a post-treated succinimide, a post-treated succinimide, or a post-treated succinate ester.

35. The process of claim 34, wherein the post-treating agent comprises at least one of urea, thiourea, carbon disulfide, aldehydes, ketones, carboxylic acids, hydrocarbon-substituted succinic anhydrides, nitriles, epoxides, boron compounds, phosphorus compounds, 2,5-dimercaptothiadiazole, or an aromatic diacid having acid groups in 1,3 or 1,4 positions on a benzene ring.

36. (canceled)

37. (canceled)

38. (canceled)

39. The process of claim 21, wherein the radically-functionalized PIBSA product is a product of a process comprising:
reacting polyisobutylene with a radical initiator and an ethylenically unsaturated acylating agent at a reaction temperature of from 150 °C to 225 °C to produce the product;
wherein the polyisobutylene is brought to the reaction temperature prior to addition of the radical initiator; and
wherein the ethylenically unsaturated acylating agent is added at any time prior to or during addition of the radical initiator.

40. The process of claim 39, wherein the ethylenically unsaturated acylating agent is combined with the polyisobutylene at the beginning of the process.

41. The process of claim 39, wherein the ethylenically unsaturated acylating agent is combined with the radical initiator for addition to the polyisobutylene after the polyisobutylene is brought to the reaction temperature.

42. The process of claim 39, wherein the radically-functionalized PIBSA product comprises less than 15 weight percent of a material comprising two or more polyisobutylene units linked by one or more acylating agent units.

43. The process of claim 39, wherein the polyisobutylene comprises an average of 3 to 56 isobutylene monomeric units per molecule.

44. The process of claim 39, wherein the radical initiator comprises an organic peroxide initiator or a peracid initiator.

45. The process of claim 44, wherein the organic peroxide initiator comprises diglutaroyl, dilauroyl peroxide, benzoyl peroxide, dicumyl peroxide, di(tert butyl) peroxide, tertbutyl hydroperoxide, 3,4-dimethyl-3,4-diphenyl-hexane, tert-butylperoxy-2-ethylhexanoate, tert-butylperoxy-benzoate, tert-butylperoxy-pivalate, tert-butylperoxy-3,5,5-trimethyl-hexanoate, di(2,4-dichlorobenzoyl) peroxide, di(2-methylbenzoyl) peroxide, or di(4-tert-butylcyclohexyl) peroxydicarbonate.

46. The process of claim 39, wherein the ethylenically unsaturated acylating agent comprises maleic acid, fumaric acid, itaconic acid, mesaconic acid, methylenemalonic acid, citraconic acid, maleic anhydride, itaconic anhydride, citraconic anhydride, or methylenemalonic anhydride.

47. The process of claim 39, wherein the radically-functionalized PIBSA product comprises at least one compound of the following general formula I: 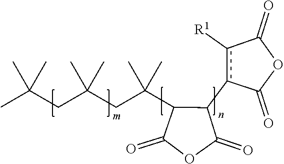
wherein:
each R¹ independently represents: —(CH₂)—(C(CH₂))—(CH₃); H; or is not present; wherein when R¹ is not present, the dashed double bond represents a double bond, and when R¹ is present, the dashed double bond represents a single bond;
each m independently is an integer from 1 to 52; and
each n independently is 0, 1 or 2.

48. The process of claim 47, wherein the radically-functionalized PIBSA product comprises at least 30 mol % of the at least one compound of the general formula I.

49. The process of claim 39, wherein the radically-functionalized PIBSA product comprises less than 40 weight percent unreacted polyisobutylene.

50. A composition comprising the reaction product of the process of claim 21.

51. A lubricant composition comprising an oil of lubricating viscosity and the composition of claim 50.

52. (canceled)

53. A method of lubricating a mechanical device comprising supplying the lubricant composition of claim 51 to the mechanical device.
